(19) United States
(12) Patent Application Publication
(10) Pub. No.: US 2007/0043076 A1
(43) Pub. Date: Feb. 22, 2007

(54) Substituted 2-arylmethylene-n-aryl-n'aryl-malonamides and analogs as activators of caspases and inducers of apoptosis

(76) Inventors: Sui Xiong Cai, San Diego, CA (US); Azra Pervin, Murrieta, CA (US); Shailaja Kasibhatla, San Diego, CA (US); Bao Ngoc Nguyen, San Diego, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/572,910

(22) PCT Filed: Oct. 5, 2004
(86) PCT No.: PCT/US04/32570

§ 371 Date: Mar. 21, 2006

Related U.S. Application Data
(60) Provisional application No. 60/508,290, filed on Oct. 6, 2003.

(51) Int. Cl. A61K 31/4709; A61K 31/444; A61K 31/416; A61K 31/4178; A61K 31/405; A61K 31/381; A61K 31/4025; A61K 31/165

(52) U.S. Cl. 514/307; 514/313; 514/406; 514/332; 514/419; 514/397; 514/471; 514/459; 514/616; 514/422

(57) ABSTRACT

The present invention is directed to substituted 2-arylmethylene-N-aryl-N′-aryl-malonamides and analogs thereof. The present invention also relates to the discovery that the compounds are activators of caspases and inducers of apoptosis. Therefore, the activators os caspases and inducers of apoptosis of this invention can be used to induce cell death in a variety of clinical conditions in which uncontrolled growth and spread of abnormal cells occurs.

### BACKGROUND OF THE INVENTION

1. Field of the Invention

This invention is in the field of medicinal chemistry. In particular, the invention relates to substituted 2-arylmethylene-N-aryl-N′-aryl-malonamides and analogs, and the discovery that these compounds are activators of caspases and inducers of apoptosis. The invention also relates to the use of these compounds as therapeutically effective anti-cancer agents.

2. Description of Background Art

Organisms eliminate unwanted cells by a process variously known as regulated cell death, programmed cell death, or apoptosis. Such cell death occurs as a normal aspect of animal development, as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane-enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall, pp. 9-34 (1981)). A cell activates its internally-encoded suicide program as a result of either internal or external signals. The suicide program is executed through the activation of a carefully regulated genetic program (Wyllie, et al., Int. Rev. Cyt. 68:251 (1980); Ellis, et al., Ann. Rev. Cell Bio. 7:663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

It has been found that a group of proteases are a key element in apoptosis (see, e.g., Thornberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, 2 of which are the pro-apoptotic (death-promoting) ced (for cell death abnormal) genes, ced-3 and ced-4. CED-3 is homologous to interleukin 1 beta-converting enzyme, a cysteine protease, which is now called caspase 1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activates the caspase cascade. This makes the cancer cells lose their capacity to undergo cellular suicide so the cells become immortal—they become cancerous. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (Schmitt, et al., Biochem. Cell. Biol. 75:301-314 (1997)). BCL-like proteins include BCL-xL and BAX-alpha, which appear to function upstream of caspase activation. BCL-xL appears to prevent activation of the apoptotic protease cascade, whereas BAX-alpha accelerates activation of the apoptotic protease cascade.

It has been shown that chemotherapeutic (anti-cancer) drugs can trigger cancer cells to undergo suicide by activating the dormant caspase cascade. This may be a crucial aspect of the mode of action of most, if not all, known anticancer drugs (Los, et al., Blood 90(8):3118-3129 (1997); Friesen, et al., Nat. Med. 2:574 (1996)). The mechanism of action of current antineoplastic drugs frequently involves an attack at specific phases of the cell cycle. In brief, the cell cycle refers to the stages through which cells normally progress during their lifetime. Normally, cells exist in a resting phase termed Gₒ. During multiplication, cells progress to a stage in which DNA synthesis occurs, termed S. Later, cell division, or mitosis, occurs in a phase called M. Antineoplastic drugs, such as cytosine arabinoside, hydroxyurea, 6-mercaptopurine, and methotrexate are S phase specific, whereas antineoplastic drugs, such as vincristine, vinblastine, and paclitaxel are M phase specific. Many slow-growing tumors, e.g. colon cancers, exist primarily in the Gₒ phase, whereas rapidly proliferating normal tissues, e.g. bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, et al., eds., Goodman and Gilman's The Pharmacological Basis of Therapeutics, Ninth Edition, McGraw-Hill, New York, pp. 1225-1287 (1996)). Thus, it is clear that the possibility exists for the activation of the caspase cascade, although the exact mechanisms for doing so are not clear at this point. It is equally clear that insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could also involve the enhancement of the apoptotic process through the administration of appropriate caspase cascade activators and inducers of apoptosis.

U.S. Pat. No. 4,634,777 discloses [(1,3-dioxo-1,3-propanediyl)diamino)]-bisbenzoic acid derivatives, with one group of the compounds having the following formula: 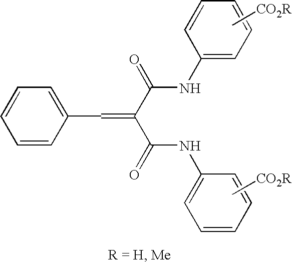
The compounds are said to be hayluronidase inhibitors and useful as anti-allergic and anti-ulcer agents.

### SUMMARY OF THE INVENTION

The present invention is related to the discovery that substituted 2-arylmethylene-N-aryl-N′-aryl-malonamide and analogs, as represented in Formulae I-II, are activators of the caspase cascade and inducers of apoptosis. Therefore, the first aspect of the present invention is directed to the use of compounds of Formulae I-II as inducers of apoptosis.

A second aspect of the present invention is to provide a method for treating, preventing or ameliorating neoplasia and cancer by administering a compound of Formulae I-II to a mammal in need of such treatment.

A third aspect of the present invention is to provide novel compounds of Formulae I-II, and to also provide for the use of these novel compounds for treating, preventing or ameliorating neoplasia and/or cancer.

A fourth aspect of the present invention is to provide a pharmaceutical composition useful for treating disorders responsive to the induction of apoptosis, containing an effective amount of a compound of Formulae I-II in admixture with one or more pharmaceutically acceptable carriers or diluents.

A fifth aspect of the present invention is directed to methods for the preparation of novel compounds of Formulae I-II.

### BRIEF DESCRIPTION OF THE DRAWINGS

FIGS. 1A-C are graphs showing drug induced apoptosis in Jurkat cells. FIG. 1A: control cells (DMSO treated) showing most of the cells in G1 (M2). FIG. 1B: cells treated with 0.5 μM of N,N′-bis-(3-trifluoromethyl-phenyl)-2-(4-isopropyl-benzylidene)-malonamide for 24 h showing 50% of the cells in M1 (subdiploid apoptotic cells). FIG. 1C: cells treated with 1 μM of N,N′-bis-(3-trifluoromethyl-phenyl)-2-(4-isopropyl-benzylidene)-malonamide for 24 h showing 77% of the cells in M1 (subdiploid apoptotic cells).

FIGS. 2A-B are graphs showing drug induced apoptosis in T47D cells. FIG. 2A: control cells (DMSO treated) showing most of the cells in G1 (M2). FIG. 2B: cells treated with 1.7 μM of N,N′-bis-(3-trifluoromethyl-phenyl)-2-(4-isopropyl-benzylidene)-malonamide for 24 h showing 88% of the cells in M1 (subdiploid apoptotic cells).

### DETAILED DESCRIPTION OF THE INVENTION

The present invention arises out of the discovery that substituted 2-arylmethylene-N-aryl-N′-aryl-malonamide and analogs are potent and highly efficacious activators of the caspase cascade and inducers of apoptosis. Therefore, these compounds are useful for treating disorders responsive to induction of apoptosis.

Specifically, compounds useful in this aspect of the present invention are substituted 2-arylmethylene-N-aryl-N′-aryl-malonamides and analogs as represented by Formula I: 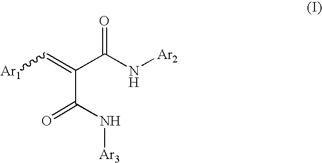
and pharmaceutically acceptable salts and prodrugs thereof, wherein: Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

Preferably Ar₁, Ar₂ and Ar₃ are optionally substituted phenyl, naphthyl, pyridyl, quinolyl, isoquinolyl, thienyl, furyl, pyrrolyl, indolyl, imidazolyl, pyrazolyl, or cyclohexyl. More preferably Ar₁, Ar₂ and Ar₃ are optionally substituted phenyl or pyridyl. More preferably at least one of the Ar₁, Ar₂ and Ar₃ is an optionally substituted pyridyl.

One embodiment of the present invention is directed to compounds of Formula II: 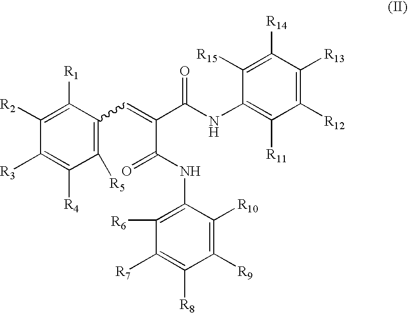
and pharmaceutically acceptable salts and prodrugs thereof, wherein R₁-R₁₅ are independently hydrogen, halo, haloalkyl, aryl, optionally substituted fused aryl, optionally substituted fused heteroaryl, carbocyclic, a heterocyclic group, a heteroaryl group, optionally substituted alkyl, alkenyl, alkynyl, arylalkyl, arylalkenyl, arylalkynyl, heteroarylalkyl, heteroarylalkenyl, heteroarylalkynyl, carbocyclealkyl, heterocycloalkyl, hydroxyalkyl, nitro, amino, cyano, acylamido, hydroxy, thiol, acyloxy, azido, alkoxy, carboxy, carbonylamido, alkylthiol, sulfonyl, alkylsulfonyl or alkylcarboxylate.

Preferred compounds falling within the scope of Formula II include compounds wherein R₁-R₁₅ are independently hydrogen, halo, haloalkyl, aryl, optionally substituted fused heteroaryl, carbocyclic, a heterocyclic group, a heteroaryl group, optionally substituted alkyl, alkenyl, alkynyl, arylalkyl, arylalkenyl, arylalkynyl, heteroarylalkyl, heteroarylalkenyl, heteroarylalkynyl, carbocyclealkyl, heterocycloalkyl, hydroxyalkyl, nitro, amino, cyano, acylamido, hydroxy, thiol, acyloxy, azido, alkoxy, carboxy, carbonylamido, alkylthiol, sulfonyl, alkylsulfonyl or alkylcarboxylate. More preferably, R₁-R₁₅ are independently hydrogen, halo, haloalkyl, aryl, carbocyclic, a heterocyclic group, a heteroaryl group, optionally substituted alkyl, alkenyl, alkynyl, arylalkyl, arylalkenyl, arylalkynyl, heteroarylalkyl, heteroarylalkenyl, heteroarylalkynyl, carbocyclealkyl, heterocycloalkyl, hydroxyalkyl, nitro, amino, cyano, acylamido, hydroxy, thiol, acyloxy, azido, alkoxy, carboxy, carbonylamido, alkylthiol, sulfonyl, alkylsulfonyl or alkylcarboxylate. Most preferably, one of more of the R₁-R₁₅ is an optionally substituted alkoxy group including 2-morpholin-4-yl-ethoxy, 2-(4-methyl-piperazin-1-yl)-ethoxy, 2-dimethylamino-ethoxy, 2-diethylamino-ethoxy, or one of more of the R₁-R₁₅ is an optionally substituted sulfonyl group including morpholine-4-sulfonyl, 4-methyl-piperazine-1-sulfonyl.

Exemplary preferred compounds that may be employed in the method of invention include, without limitation:
2-[3-(4-Chloro-phenyl)-1-phenyl-1H-pyrazol-4-ylmethylene]-N,N′-bis-(3-trifluoromethyl-phenyl)-malonamide;
2-[3-(4-Methoxy-phenyl)-1-phenyl-1H-pyrazol-4-ylmethylene]-N,N′-bis-(3-trifluoromethyl-phenyl)-malonamide;
N,N′-Bis-(3-trifluoromethyl-phenyl)-2-(4-methyl-benzylidene)-malonamide;
N,N′-Bis-(3-trifluoromethyl-phenyl)-2-(4-bromo-benzylidene)-malonamide;
N,N′-Bis-(3-trifluoromethyl-phenyl)-2-(4-nitro-benzylidene)-malonamide;
N,N′-Bis-(3-trifluoromethyl-phenyl)-2-(3-chloro-benzylidene)-malonamide;
2-[3-(4-Fluoro-phenyl)-1-phenyl-1H-pyrazol-4-ylmethylene]-N,N′-bis-(3-trifluoromethyl-phenyl)-malonamide;
2-[1-phenyl-3-(thiophen-2-yl)-1H-pyrazol-4-ylmethylene]-N,N′-bis-(3-trifluoromethyl-phenyl)-malonamide;
N,N′-Bis-(3-methoxy-phenyl)-2-(4-trifluoromethyl-benzylidene)-malonamide;
N,N′-Bis-(3-trifluoromethyl-phenyl)-2-(2,4-dichloro-benzylidene)-malonamide;
N,N′-Bis-(3-trifluoromethyl-phenyl)-2-(4-isopropyl-benzylidene)-malonamide;
N,N′-Bis-(3-trifluoromethyl-phenyl)-2-furan-2-yl-methylene-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Bis-(3-trifluoromethyl-phenyl)-2-(4-pyridyl-methylene)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-trifluoromethyl-phenyl)-2-(3H-imidazol-4-yl-methylene)-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(4-ethoxy-benzylidene)-malonamide;
N,N′-Bis-(3-trifluoromethyl-phenyl)-2-(5-methyl-furan-2-yl-methylene)-malonamide;
N,N′-Bis-(3-methoxy-phenyl)-2-(4-chloro-2-nitro-benzylidene)-malonamide;
N,N′-Bis-(pyridin-3-yl)-2-(4-isopropyl-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Bis-(3-trifluoromethyl-phenyl)-2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
3-(4-Isopropyl-phenyl)-N-(3-trifluoromethyl-phenyl)-2-(3-trifluoromethyl-phenylsulfamoyl)-acrylamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Chloro-pyridin-4-yl)-2-(2,3-dihydro-benzo[1,4]dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
(E) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(6-bromo-pyridin-2-yl-methylene)malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(6-bromo-pyridin-2-yl)-2-(4-isopropyl-benzylidene)-malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide;
(E) and (Z)-2-(3-Trifluoromethyl-benzylidene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide.

The present invention is also directed to novel compounds within the scope of Formulae I-II. Exemplary preferred compounds that may be employed in this
N,N′-Bis-(3-methoxy-phenyl)-2-(4-trifluoromethyl-benzylidene)-malonamide;
N,N′-Bis-(3-trifluoromethyl-phenyl)-2-furan-2-yl-methylene-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Bis-(3-trifluoromethyl-phenyl)-2-(4-pyridyl-methylene)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-trifluoromethyl-phenyl)-2-(3H-imidazol-4-yl-methylene)-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(4-ethoxy-benzylidene)-malonamide;
N,N′-Bis-(3-trifluoromethyl-phenyl)-2-(5-methyl-furan-2-yl-methylene)-malonamide;
N,N′-Bis-(3-methoxy-phenyl)-2-(4-chloro-2-nitro-benzylidene)-malonamide;
N,N′-Bis-(pyridin-3-yl)-2-(4-isopropyl-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(4 isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Bis-(3-trifluoromethyl-phenyl)-2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
3-(4-Isopropyl-phenyl)-N-(3-trifluoromethyl-phenyl)-2-(3-trifluoromethyl-phenylsulfamoyl)-acrylamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-2-(4-Isopropyl-benzylidene)-N-[4-(2-morpholinyl-4-yl-ethoxy)-3-trifluoromethyl-phenyl]-N′-(3-trifluoromethyl-phenyl)-malonamide;
(E) and (Z)-N-(2-Chloro-pyridin-4-yl)-2-(2,3-dihydro-benzo[1,4]dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
(E) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(6-bromo-pyridin-2-yl-methylene)malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(6-bromo-pyridin-2-yl)-2-(4-isopropyl-benzylidene)-malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide;
(E) and (Z)-2-(3-Trifluoromethyl-benzylidene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide.

Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which can be optionally substituted.

Useful alkenyl groups include straight-chained and branched C₁₋₁₀ alkenyl groups, more preferably C₁₋₆ alkenyl groups. Typical C₁₋₁₀ alkenyl groups include ethenyl, propenyl, isopropenyl, butenyl, sec-butenyl, 3-pentenyl, hexenyl and octenyl groups, which can be optionally substituted.

Useful alkynyl groups include straight-chained and branched C₁₋₁₀ alkynyl groups, more preferably C₁₋₆ alkynyl groups. Typical C₁₋₁₀ alkynyl groups include ethynyl, propynyl, butynyl, 3-pentynyl, hexynyl and octynyl groups, which can be optionally substituted.

Useful alkoxy groups include oxygen substituted by one of the C₁₋₁₀ alkyl groups mentioned above, which can be optionally substituted.

Useful alkylthio groups include sulphur substituted by one of the C₁₋₁₀ alkyl groups mentioned above, which can be optionally substituted. Also included are the sulfoxides and sulfones of such alkylthio groups.

Useful amino groups include —NH₂, —NHR₁₁, and —NR₁₁R₁₂, wherein R₁₁ and R₁₂ are C₁₋₁₀ alkyl or cycloalkyl groups, aryl or heteroaryl groups, or arylalkyl or heteroarylalkyl groups, or R₁₁ and R₁₂ are combined with the N to form a cycloamino structure, such as a piperidine, or R₁₁ and R₁₂ are combined with the N and other groups to form a cycloamino structure, such as a piperazine. The alkyl, cycloalkyl, aryl, heteroaryl, cycloamino groups can be optionally substituted.

Optional substituents on the alkyl groups include one or more halo, hydroxy, carboxyl, amino, nitro, cyano, C₁-C₆ acylamino, C₁-C₆ acyloxy, C₁-C₆ alkoxy, aryloxy, alkylthio, C₆-C₁₀ aryl, C₄-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated and unsaturated heterocyclic, or heteroaryl. Optional substituents on the aryl, aralkyl and heteroaryl groups include one or more halo, C₁-C₆ haloalkyl, C₆-C₁₀ aryl, heteroaryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy, carboxy, (C₁-C₆)alkylsulfonyl and (C₁-C₆)alkylcarboxylate.

Useful aryl groups are C₆₋₁₄ aryl, especially C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as defined above, as well as cycloalkenyl groups, such as cyclopentenyl, cycloheptenyl and cyclooctenyl.

Useful halo or halogen groups include fluorine, chlorine, bromine and iodine.

Useful arylalkyl groups include any of the above-mentioned C₁₋₁₀ alkyl groups substituted by any of the above-mentioned C₆₋₁₄ aryl groups. Useful values include benzyl, phenethyl and naphthylmethyl.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino groups are any C₁₋₆ acyl (alkanoyl) attached to an amino nitrogen, e.g., acetamido (acetylamino), propionamido, butanoylamido, pentanoylamido, hexanoylamido, as well as aryl-substituted C₂₋₆ substituted acyl groups.

Useful acyloxy groups are any C₁₋₆ acyl (alkanoyl) attached to an oxy (—O—) group, e.g., formyloxy, acetoxy, propionoyloxy, butanoyloxy, pentanoyloxy, hexanoyloxy and the like.

Useful saturated or partially saturated heterocyclic groups include tetrahydrofuranyl, pyranyl, piperidinyl, piperazinyl, 4-methyl-piperazinyl, 4-pyridyl-piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

Useful heteroaryl groups include any one of the following: thienyl, benzo[b]thienyl, naphtho[2,3-b]thienyl, thianthrenyl, furanyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]-pyrimidin-4-one, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g. a pyridyl N-oxide, pyrazinyl N-oxide, pyrimidinyl N-oxide and the like.

Certain of the compounds of the present invention may exist as stereoisomers including optical isomers. The invention includes all stereoisomers and both the racemic mixtures of such stereoisomers, as well as the individual enantiomers that may be separated according to methods that are well known to those of ordinary skill in the art.

Examples of pharmaceutically acceptable addition salts include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine.

Examples of prodrugs of the compounds of the invention include the simple esters of carboxylic acid containing compounds (e.g. those obtained by condensation with a C₁₋₄ alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

The compound of this invention may be prepared using methods known to those skilled in the art, or novel method of this invention. Specifically, compounds of formula I wherein Ar₂ and Ar₃ are the same can be prepared as illustrated by exemplary reaction in Scheme 1 in two steps. Reaction of 3-aminobenzotrifluoride with malonyl dichloride in dioxane gives the intermediate, N,N′-bis-(3-trifluoromethyl-phenyl)-malonamide. Reaction of N,N′-bis-(3-trifluoromethyl-phenyl)-malonamide with 4-isopropylbenzaldehyde in anhydrous tetrahydrofuran (THF) in the presence of catalytic amount of piperidine and glacial acetic acid produces N,N′-bis-(3-trifluoromethyl-phenyl)-2-(4-isopropylbenzylidene)-malonamide.

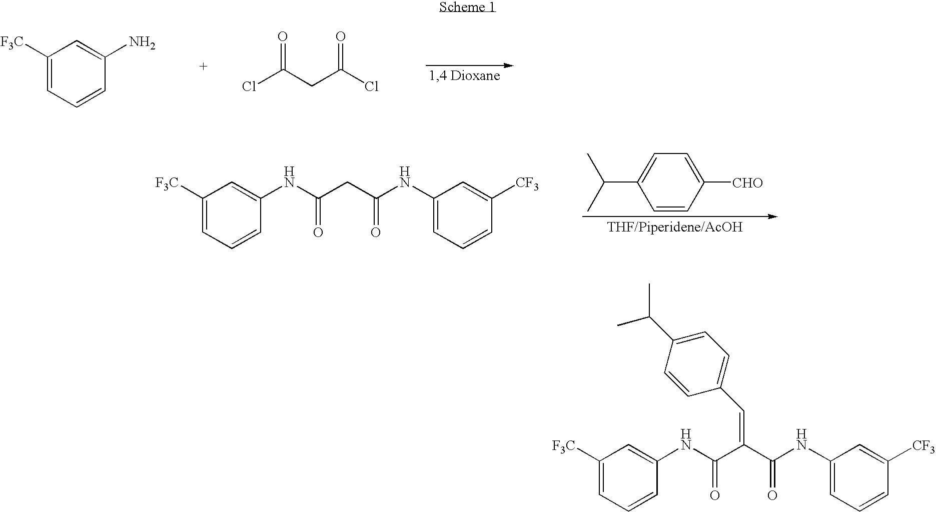

Compounds of formula I wherein Ar₂ and Ar₃ are not the same can be % prepared as illustrated by exemplary reaction in scheme 2. Reaction of trifluoromethyl aniline in anhydrous DCM with methyl malonyl chloride gives N-(3-trifluoromethyl-phenyl)-malonamic acid methyl ester. The ester is hydrolyzed in 2N NaOH to N-(3-trifluoromethyl-phenyl)-malonamic acid. Treatment of N-(3-trifluoromethyl-phenyl)-malonamic acid with PCl₅ and aniline produces the intermediate N-phenyl-N′-(3-trifluoromethyl-phenyl)-malonamide. Reaction of N-phenyl-N′-(3-trifluoromethyl-phenyl)-malonamide and 4-isopropylbenzaldehyde in anhydrous tetrahydrofuran in presence of catalytic amount of piperidine and glacial acetic acid give the E and Z isomers of 2-(4-isopropyl-benzylidene)-N-phenyl-N′-(3-trifluoromethyl-phenyl)-malonamide.

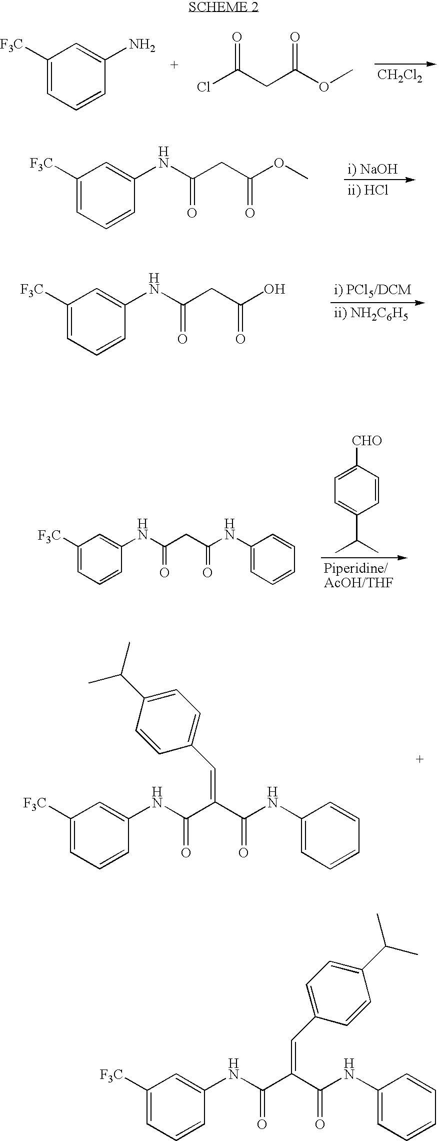

An important aspect of the present invention is the discovery that compounds having Formulae I-II are activators of caspases and inducers of apoptosis. Therefore, these compounds are useful in a variety of clinical conditions in which there is uncontrolled cell growth and spread of abnormal cells, such as in the case of cancer.

Yet another important aspect of the present invention is the discovery that the compounds described herein are potent and highly efficacious activators of caspases and inducers of apoptosis in drug-resistant cancer cells, such as breast and prostate cancer cells, which enables these compounds to kill drug-resistant cancer cells. In comparison, most standard anti-cancer drugs are not effective in killing drug-resistant cancer cells under the same conditions. Therefore, compounds having Formulae I-II are expected to be useful for the treatment of drug-resistant cancer in animals.

The present invention includes a therapeutic method useful to modulate in vivo apoptosis or in vivo neoplastic disease, comprising administering to a subject in need of such treatment an effective amount of a compound, or a pharmaceutically acceptable salt or prodrug of a compound described herein, which functions as a caspase cascade activator and inducer of apoptosis.

The present invention also includes a therapeutic method comprising administering to an animal an effective amount of a compound, or a pharmaceutically acceptable salt or prodrug of said compound of Formulae I-II, wherein said therapeutic method is useful to treat cancer, which is a group of diseases characterized by the uncontrolled growth and spread of abnormal cells. Such diseases include, but are not limited to, Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

In practicing the therapeutic methods, effective amounts of compositions containing therapeutically effective concentrations of the compounds formulated for oral, intravenous, local and topical application (for the treatment of neoplastic diseases and other diseases in which caspase cascade mediated physiological responses are implicated), are administered to an individual exhibiting the symptoms of one or more of these disorders. The amounts are effective to ameliorate or eliminate one or more symptoms of the disorder. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In another embodiment, a pharmaceutical composition comprising a compound, or a pharmaceutically acceptable salt of a compound described herein, which functions as a caspase cascade activator and inducer of apoptosis in combination with a pharmaceutically acceptable vehicle, is provided.

Another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cancer chemotherapeutic agent, or a pharmaceutically acceptable salt of said agent. Examples of known anti-cancer agents which can be used for combination therapy include, but are not limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; and antibodies, such as Herceptin® and Rituxan®. Other known anti-cancer agents, which can be used for combination therapy, include arsenic trioxide, gamcitabine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In practicing the methods of the present invention, the compound of the invention may be administered together with the at least one known chemotherapeutic agent as part of a unitary pharmaceutical composition. Alternatively, the compound of the invention may be administered apart from the at least one known cancer chemotherapeutic agent. In this embodiment, the compound of the invention and the at least one known cancer chemotherapeutic agent are administered substantially simultaneously, i.e., the compounds are administered at the same time or one after the other, so long as the compounds reach therapeutic levels for a period of time in the blood.

It has been reported that alpha-1-adrenoceptor antagonists, such as doxazosin, terazosin, and tamsulosin, can inhibit the growth of prostate cancer cell via induction of apoptosis (Kyprianou, N., et al., Cancer Res. 60:4550-4555 (2000)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known alpha-1-adrenoceptor antagonists, or a pharmaceutically acceptable salt of said agent. Examples of known alpha-1-adrenoceptor antagonists, which can be used for combination therapy include, but are not limited to, doxazosin, terazosin, and tamsulosin.

It has been reported that sigma-2 receptors are expressed in high densities in a variety of tumor cell types (Vilner, B. J., et al., Cancer Res. 55: 408-413 (1995)) and that sigma-2 receptor agonists, such as CB-64D, CB-184 and haloperidol activate a novel apoptotic pathway and potentiate antineoplastic drugs in breast tumor cell lines (Kyprianou, N., et al., Cancer Res. 62:313-322 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known sigma-2 receptor agonists, or a pharmaceutically acceptable salt of said agent. Examples of known sigma-2 receptor agonists, which can be used for combination therapy include, but are not limited to, CB-64D, CB-184 and haloperidol.

It has been reported that combination therapy with lovastatin, a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HMG-CoA reductase inhibitor, or a pharmaceutically acceptable salt of said agent. Examples of known HMG-CoA reductase inhibitors, which can be used for combination therapy include, but are not limited to, lovastatin, simvastatin, pravastatin, fluvastatin, atorvastatin and cerivastatin.

It has been reported that HIV protease inhibitors, such as indinavir or saquinavir, have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HIV protease inhibitor, or a pharmaceutically acceptable salt of said agent. Examples of known HIV protease inhibitors, which can be used for combination therapy include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

It has been reported that synthetic retinoids, such as fenretinide (N-(4-hydroxyphenyl)retinamide, 4HPR), have good activity in combination with other chemotherapeutic agents, such as cisplatin, etoposide or paclitaxel in small-cell lung cancer cell lines Kalemkerian, G. P., et al., Cancer Chemother. Pharmacol. 43:145-150 (1999)). 4HPR also was reported to have good activity in combination with gamma-radiation on bladder cancer cell lines (Zou, C., et al., Int. J. Oncol. 13:1037-1041 (1998)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known retinoid and synthetic retinoid, or a pharmaceutically acceptable salt of said agent. Examples of known retinoids and synthetic retinoids, which can be used for combination therapy include, but are not limited to, bexarotene, tretinoin, 13-cis-retinoic acid, 9-cis-retinoic acid, α-difluoromethylornithine, ILX23-7553, fenretinide, and N-4-carboxyphenyl retinamide.

It has been reported that proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known proteasome inhibitor, or a pharmaceutically acceptable salt of said agent. Examples of known proteasome inhibitors, which can be used for combination therapy include, but are not limited to, lactacystin, MG-132, and PS-341.

It has been reported that tyrosine kinase inhibitors, such as STI571 (Imatinib mesilate, Gleevec®), have potent synergetic effect in combination with other anti-leukemic agents, such as etoposide (Liu, W. M., et al., Br. J. Cancer 86:1472-1478 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known tyrosine kinase inhibitor, or a pharmaceutically acceptable salt of said agent. Examples of known tyrosine kinase inhibitors, which can be used for combination therapy include, but are not limited to, Gleevec®, ZD1839 (Iressa), SH268, genistein, CEP2563, SU6668, SU11248, and EMD121974.

It has been reported that prenyl-protein transferase inhibitors, such as farnesyl protein transferase inhibitor R115777, possess preclinical antitumor activity against human breast cancer (Kelland, L. R., et. al., Clin. Cancer Res. 7:3544-3550 (2001)). Synergy of the protein farnesyltransferase inhibitor SCH66336 and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of said agent. Examples of known prenyl-protein transferase inhibitors, which can be used for combination therapy include, but are not limited to, R115777, SCH66336, L-778,123, BAL9611 and TAN-1813.

It has been reported that cyclin-dependent kinase (CDK) inhibitors, such as flavopiridol, have potent synergetic effect in combination with other anticancer agents, such as CPT-11, a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cyclin-dependent kinase inhibitor, or a pharmaceutically acceptable salt of said agent. Examples of known cyclin-dependent kinase inhibitor, which can be used for combination therapy include, but are not limited to, flavopiridol, UCN-01, roscovitine and olomoucine.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16 (4:3):17-21 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known COX-2 inhibitor, or a pharmaceutically acceptable salt of said agent. Examples of known COX-2 inhibitors, which can be used for combination therapy include, but are not limited to, celecoxib, valecoxib, and rofecoxib.

Another embodiment of the present invention is directed to a composition effective to inhibit neoplasia comprising a bioconjugate of a compound described herein, which functions as a caspase cascade activator and inducer of apoptosis, in bioconjugation with at least one known therapeutically useful antibody, such as Herceptin® or Rituxan®, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates could also enhance the anticancer effect of therapeutically useful antibodies, such as Herceptin® or Rituxan®.

Similarly, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a caspase cascade activator and inducer of apoptosis. The invention also relates to a method of treating cancer by surgically removing the cancer and then treating the animal with one of the pharmaceutical compositions described herein.

A wide range of immune mechanisms operate rapidly following exposure to an infectious agent. Depending on the type of infection, rapid clonal expansion of the T and B lymphocytes occurs to combat the infection. The elimination of the effector cells following an infection is one of the major mechanisms maintaining immune homeostasis. This deletion of reactive cells has been shown to be regulated by a phenomenon known as apoptosis. Autoimmune diseases have been lately identified as a consequence of deregulated cell death. In certain autoimmune diseases, the immune system directs its powerful cytotoxic effector mechanisms against specialized cells, such as oligodendrocytes in multiple sclerosis, the beta cells of the pancreas in diabetes mellitus, and thyrocytes in Hashimoto's thyroiditis (Ohsako, S., et al., Cell Death Differ. 6(1):13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly and generalized lymphadenopathy, hypergammaglobulinemia, and autoantibody formation (Infante, A. J., et al., J. Pediatr. 133(5):629-633 (1998) and Vaishnaw, A. K, et al., J. Clin. Invest. 103(3):355-363 (1999)). It was reported that overexpression of Bcl-2, which is a member of the Bcl-2 gene family of programmed cell death regulators with anti-apoptotic activity, in developing B cells of transgenic mice, in the presence of T cell dependent costimulatory signals, results in the generation of a modified B cell repertoire and in the production of pathogenic autoantibodies (Lopez-Hoyos, M., et al., Int. J. Mol. Med. 1(2):475-483 (1998)). Therefore, it is evident that many types of autoimmune disease are caused by defects of the apoptotic process and one treatment strategy would be to turn on apoptosis in the lymphocytes that are causing autoimmune disease (O'Reilly, L. A. and Strasser, A., Inflamm. Res. 48(1):5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al., J. Immunol. 162(1):603-608 (1999), reported that by direct injection of DNA expression vectors encoding FasL into the inflammed thyroid, the development of lymphocytic infiltration of the thyroid was inhibited and induction of the death of infiltrating T cells was observed. These results show that FasL expression on thyrocytes may have a curative effect on ongoing EAT by inducing death of pathogenic autoreactive infiltrating T lymphocytes.

Bisindolylmaleimide VIII is known to potentiate Fas-mediated apoptosis in human astrocytoma 1321N1 cells and in Molt-4T cells, both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. Zhou, T., et al., Nat. Med. 5(1):42-8 (1999), reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore, the application of a Fas-dependent apoptosis enhancer, such as bisindolylmaleimide VI, may be therapeutically useful for the more effective elimination of detrimental cells and inhibition of T cell-mediated autoimmune diseases. Therefore, an effective amount of a compound, or a pharmaceutically acceptable salt or prodrug of a compound described herein, which functions as a caspase cascade activator and inducer of apoptosis, should be an effective treatment for autoimmune disease.

Psoriasis is a chronic skin disease, which is characterized by scaly red patches. Psoralen plus ultraviolet A (PUVA) is a widely-used and effective treatment for psoriasis vulgaris. Coven, T. R., et al., Photodermatol. Photoimmunol. Photomed. 15(1):22-7 (1999), reported that lymphocytes treated with psoralen 8-MOP or TMP plus UVA displayed DNA degradation patterns typical of apoptotic cell death. Ozawa, M., et al., J. Exp. Med. 189(4):711-718 (1999), reported that induction of T cell apoptosis could be the main mechanism by which 312-nm UVB resolves psoriasis skin lesions. Low doses of methotrexate may be used to treat psoriasis to restore a clinically normal skin. Heenen, M., et al., Arch. Dermatol. Res. 290(5):240-245 (1998), reported that low doses of methotrexate may induce apoptosis and this mode of action could explain the reduction in epidermal hyperplasia during treatment of psoriasis with methotrexate. Therefore, an effective amount of a compound, or a pharmaceutically acceptable salt or prodrug of a compound described herein, which functions as a caspase cascade activator and inducer of apoptosis, should be an effective treatment for psoriasis.

Synovial cell hyperplasia is a characteristic of patients with rheumatoid arthritis (RA). Excessive proliferation of RA synovial cells that, in addition, are defective in synovial cell death might be responsible for the synovial cell hyperplasia. Wakisaka, S., et al., Clin. Exp. Immunol. 114(1):119-28 (1998), found that, although RA synovial cells could die via apoptosis through Fas/FasL pathway, apoptosis of synovial cells was inhibited by proinflammatory cytokines present within the synovium, and suggested that inhibition of apoptosis by the proinflammatory cytokines may contribute to the outgrowth of synovial cells and lead to pannus formation and the destruction of joints in patients with RA. Therefore, an effective amount of a compound, or a pharmaceutically acceptable salt or prodrug of a compound described herein, which functions as a caspase cascade activator and inducer of apoptosis, should be an effective treatment for rheumatoid arthritis.

There has been an accumulation of convincing evidence that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol., 61(4):375-80 (1997)). Boirivant, M., et al., Gastroenterology 116(3):557-65 (1999), reported that lamina propria T cells isolated from areas of inflammation in Crohn's disease, ulcerative colitis, and other inflammatory states manifest decreased CD2 pathway-induced apoptosis, and that studies of cells from inflamed Crohn's disease tissue indicate that this defect is accompanied by elevated Bcl-2 levels. Therefore, an effective amount of a compound, or a pharmaceutically acceptable salt or prodrug of a compound described herein, which functions as a caspase cascade activator and inducer of apoptosis, should be an effective treatment for inflammation.

Caspase cascade activators and inducers of apoptosis may also be a desirable therapy in the elimination of pathogens, such as HIV, Hepatitis C and other viral pathogens. The long-lasting quiecence, followed by disease progression, may be explained by an anti-apoptotic mechanism of these pathogens leading to persistent cellular reservoirs of the virions. It has been reported that HIV-1 infected T leukemia cells or peripheral blood mononuclear cells (PBMCs) underwent enhanced viral replication in the presence of the caspase inhibitor Z-VAD-fink. Furthermore, Z-VAD-fink also stimulated endogenous virus production in activated PBMCs derived from HIV-1 infected asymptomatic individuals (Chinnaiyan, X, et al., Nat. Med. 3:333 (1997)). Therefore, apoptosis serves as a beneficial host mechanism to limit the spread of HIV and new therapeutics using caspase/apoptosis activators are useful to clear viral reservoirs from the infected individuals. Similarly, HCV infection also triggers anti-apoptotic mechanisms to evade the host's immune surveillance leading to viral persistence and hepatocarcinogenesis (Tai, D. I., et al., Hepatology 3:656-64 (2000)). Therefore, apoptosis inducers are useful as therapeutics for HIV and other infectious disease.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar, E., et al., Br. Med. Bull. 59:227-248 (2001)). Therefore, apoptosis inducers, which are antiproliferative, are useful as therapeutics for in-stent restenosis.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be orally administered to mammals, e.g. humans, at a dose of 0.0025 to 50 mg/kg, or an equivalent amount of the pharmaceutically acceptable salt thereof, per day of the body weight of the mammal being treated for apoptosis-mediated disorders. Preferably, approximately 0.01 to approximately 10 mg/kg is orally administered to treat or prevent such disorders. For intramuscular injection, the dose is generally approximately one-half of the oral dose. For example, a suitable intramuscular dose would be approximately 0.0025 to approximately 25 mg/kg, and most preferably, from approximately 0.01 to approximately 5 mg/kg. If a known cancer chemotherapeutic agent is also administered, it is administered in an amount which is effective to achieve its intended purpose. The amounts of such known cancer chemotherapeutic agents effective for cancer are well known to those of skill in the art.

The unit oral dose may comprise from approximately 0.01 to approximately 50 mg, preferably approximately 0.1 to approximately 10 mg of the compound of the invention. The unit dose may be administered one or more times daily as one or more tablets, each containing from approximately 0.1 to approximately 10, preferably approximately 0.25 to 50 mg of the compound or its solvates.

In a topical formulation, the compound may be present at a concentration of approximately 0.01 to 100 mg per gram of carrier.

In addition to administering the compound alone, the compounds of the invention may be administered as part of a pharmaceutical preparation containing suitable pharmaceutically acceptable carriers comprising excipients and auxiliaries, which facilitate processing of the compounds into preparations that can be used pharmaceutically. Preferably, the preparations, particularly those preparations, which can be administered orally and which can be used for the preferred type of administration, such as tablets, dragees, and capsules, and also preparations, which can be administered rectally, such as suppositories, as well as suitable solutions for administration by injection or orally, containing from approximately 0.01 to 99 percent, preferably from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the particular apoptosis inducer of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the particular apoptosis inducer of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternative, or concurrent, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active compounds with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active compound doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active compounds in the form of granules, which may be mixed with fillers, such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active compounds with a suppository base. Suitable suppository bases are, e.g. natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils, e.g., sesame oil; or synthetic fatty acid esters, e.g., ethyl oleate or triglycerides or polyethylene glycol-400 (the compounds are soluble in PEG-400). Aqueous injection suspensions may contain substances, which increase the viscosity of the suspension include, e.g., sodium carboxymethyl cellulose, sorbitol, and/or dextran. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil such as almond oil, is admixed. A typical example of such a cream is one which includes approximately: 40 parts water, 20 parts beeswax, 40 parts mineral oil, and 1 part almond oil.

Ointments may be formulated by mixing a solution of the active ingredient in a vegetable oil, such as almond oil with warm soft paraffin and allowing the mixture to cool. A typical example of such an ointment is one which includes approximately: 30% almond oil and 70% white soft paraffin by weight.

The following examples are illustrative, but not limiting, of the method and compositions of the present invention. Other suitable modifications and adaptations of the variety of conditions and parameters normally encountered in clinical therapy, and which are obvious to those skilled in the art, are within the spirit and scope of the invention.

### EXAMPLE 1

### N,N′-Bis-(3-methoxy-phenyl)-2-(4-trifluoromethyl-benzylidene)-malonamide

a) N,N′-Bis-(3-methoxy-phenyl)-malonamide: To a stirring solution of m-anisidine (120 μL, 1.07 mmol) in 1,4-dioxane (5 mL) was slowly added malonyl dichloride (35 μL, 0.355 mmol). The mixture was stirred at room temperature for 15 min. The mixture was diluted with water and the resulting solid was collected by filtration. The solid was wash with excess water and then dried under vacuum at 45° C. to give a tan solid (65 mg, 58%). ¹H NMR (CDCl₃): 8.95 (s, 2H), 7.29-7.27 (m, 2H), 7.22 (d, J=8.4 Hz, 2H), 7.10-7.07 (m, 2H), 6.73-6.69 (m, 2H), 3.80 (s, 6H), 3.54 (s, 2H).

b) N,N′-Bis-(3-methoxy-phenyl)-2-(4-trifluoromethyl-benzylidene)-malonamide: To a stirring mixture of N,N′-bis-(3-methoxy-phenyl)-malonamide (30 mg, 0.095 mmol) and 4-(trifluoromethyl)benzaldehyde (20 μL, 0.143 mmol) in ethanol (5 mL) was added piperidine (28 μL) and glacial acetic acid (16 μL). The mixture was refluxed for 20 h. The solvent was evaporated and the resulting residue was purified by preparative TLC to obtain a yellow solid (5 mg, 11%). ¹H NMR (CDCl₃): 9.84 (s, 1H), 9.02 (s, 1H), 7.82 (s, 1H), 7.47 (d, J=8.4 Hz, 2H), 7.41 (d, J=8.7 Hz, 2H), 7.29-7.19 (m, 4H), 7.09-7.00 (m, 2H), 6.78-6.69 (m, 2H), 3.81 (s, 3H), 3.75 (s, 3H).

### EXAMPLE 2

### N,N′-Bis-(3-trifluoromethyl-phenyl)-2-(2,4-dichloro-benzylidene)-malonamide and N,N′-Bis-(3-trifluoromethyl-phenyl)-2-[(2,4-dichloro-phenyl)-ethoxy-methyl]-malonamide

a) N,N′-Bis-(3-trifluoromethyl-phenyl)-malonamide: The title compound was prepared from a mixture of 3-aminobenzotrifluoride (2.2 mL, 17.8 mmol) and malonyl dichloride (691 μL, 7.10 mmol) similar to Example 1a and isolated as an tan solid (2.56 g, 92%). ¹H NMR (CDCl₃): 9.49 (s, 2H), 7.99 (s, 2H), 7.74-7.70 (m, 2H), 7.50-7.41 (m, 4H), 3.67 (s, 2H).

b) N,N′-Bis-(3-trifluoromethyl-phenyl)-2-(2,4-dichloro-benzylidene)-malonamide: The title compound was prepared from a mixture of N,N′-bis-(3-trifluoromethyl-phenyl)-malonamide (50 mg, 0.128 mmol) and 2,4-dichlorobenzaldehyde (34 mg, 0.192 mmol) similar to Example 1b and isolated as a yellow solid (10 mg, 14%). ¹H NMR (CDCl₃): 10.41 (s, 1H), 10.28 (s, 1H), 8.01 (s, 2H), 7.86 (s, 1H), 7.75-7.68 (m, 2H), 7.53-7.48 (m, 4H), 7.35 (d, J=8.7 Hz, 1H), 7.07 (dd, J=2.4, 8.7 Hz, 1H), 6.89 (d, J=2.1 Hz, 1H).

c) N,N′-Bis-(3-trifluoromethyl-phenyl)-2-[(2,4-dichloro-phenyl)-ethoxy-methyl]-malonamide: The title compound was prepared from a mixture of N,N′-bis-(3-trifluoromethyl-phenyl)-malonamide (50 mg, 0.128 mmol) and 2,4-dichlorobenzaldehyde (34 mg, 0.192 mmol) similar to Example 1b and isolated as a yellow solid (6 mg, 8%). ¹H NMR (CDCl₃): 9.60 (s, 1H), 9.41 (s, 1H), 7.88 (s, 1H), 7.84 (s, 1H), 7.65-7.60 (m, 2H), 7.51-7.41 (m, 4H), 7.35 (d, J=8.4 Hz, 1H), 7.19 (d, J=1.8 Hz, 1H), 7.08 (dd, J=2.1, 8.4 Hz, 1H), 5.41 (d, J=7.2 Hz, 1H), 3.76 (d, J=7.2 Hz, 1H), 3.46-3.38 (m, 2H), 1.13 (t, J=6.6 Hz, 3H).

### EXAMPLE 3

### N,N′-Bis-(3-trifluoromethyl-phenyl)-2-(4-isopropyl-benzylidene)-malonamide

To a stirring mixture of N,N′-bis-(3-trifluoromethyl-phenyl)-malonamide (300 mg, 0.769 mmol) and 4-isopropylbenzaldehyde (140 μL, 0.923 mmol) in anhydrous THF (10 mL) was added piperidine (76 μL) and glacial acetic acid (44 μL). The mixture was refluxed for 1 h. The solvent was evaporated and the resulting residue was purified by column chromatography to obtain a white solid (110 mg, 28%). ¹H NMR (CDCl₃): 10.14 (s, 1H), 8.56 (s, 1H), 8.02 (s, 1H), 7.97 (s, 1H), 7.79-7.77 (m, 1H), 7.73 (s, 1H), 7.62-7.59 (m, 1H), 7.50-7.39 (m, 4H), 7.33 (d, J=8.1 Hz, 1H), 7.13 (d, J=8.1 Hz, 2H), 2.84-2.77 (m, 1H), 1.17 (s, 3H), 1.14 (s, 3H).

### EXAMPLE 4

### N,N′-Bis-(3-trifluoromethyl-phenyl)-2-furan-2-yl-methylene-malonamide

The title compound was prepared from a mixture of N,N′-bis-(3-trifluoromethyl-phenyl)-malonamide (50 mg, 0.128 mmol) and furfural (16 μL, 0.192 mmol) similar to Example 3 and isolated as a yellow solid (23 mg, 38%). ¹H NMR (CDCl₃): 10.16 (s, 1H), 10.01 (s, 1H), 8.28 (s, 1H), 7.98-7.94 (m, 2H), 7.54 (s, 1H), 7.59-7.31 (m, 6H), 6.68 (d, J=3.6 Hz, 1H), 6.23 (dd, J=1.8, 3.6 Hz, 1H).

### EXAMPLE 5

### N,N′-Bis-(3-trifluoromethyl-phenyl)-2-(2,4-dimethyl-benzylidene)-malonamide

The title compound was prepared from a mixture of N,N′-bis-(3-trifluoromethyl-phenyl)-malonamide (50 mg, 0.128 mmol) and 2,4-dimethylbenzaldehyde (27 μL, 0.192 mmol) similar to Example 3 and isolated as a white solid (13 mg, 20%). ¹H NMR (CDCl₃): 10.33 (s, 1H), 8.59 (s, 1H), 8.23 (s, 1H), 8.00 (s, 1H), 7.72 (d, J=8.1 Hz, 1H), 7.63 (s, 1H), 7.57-7.54 (m, 1H), 7.48-7.39 (m, 4H), 7.23 (d, J=8.4 Hz, 1H), 6.92 (s, 1H), 6.90 (s, 1H), 2.29 (s, 3H), 2.24 (s, 3H).

### EXAMPLE 6

### N,N′-Bis-(3-trifluoromethyl-phenyl)-2-(2,4-dimethoxy-benzylidene)-malonamide

The title compound was prepared from a mixture of N,N′-bis-(3-trifluoromethyl-phenyl)-malonamide (50 mg, 0.128 mmol) and 2,4dimethoxybenzaldehyde (26 mg, 0.154 mmol) similar to Example 3 and isolated as a yellow solid (37 mg, 54%). ¹H NMR (CDCl₃): 10.56 (s, 1H), 10.37 (s, 1H), 8.21 (s, 1H), 8.09 (s, 1H), 8.02 (s, 1H), 7.88 (d, J=7.8 Hz, 1H), 7.65 (d, J=7.8 Hz, 1H), 7.55-7.40 (m, 4H), 7.29 (d, J=8.7 Hz, 1H), 6.33 (dd, J=2.4, 8.7 Hz, 1H), 5.98 (d, J=2.1 Hz, 1H), 3.66 (s, 3H), 3.52 (s, 3H).

### EXAMPLE 7

### N,N′-Bis-(3-trifluoromethyl-phenyl)-2-(4-isopropyl-benzyl)-malonamide

To a solution of N,N′-bis-(3-trifluoromethyl-phenyl)-2-(4-isopropyl-benzylidene)-malonamide (25 mg, 0.048 mmol) in methanol (20 mL) was added palladium (5 wt % on activated carbon). The mixture was hydrogenated at 50 psi for 20 h. The mixture was filtered over celite and washed with methanol. The solvent was evaporated to dryness to obtain a white solid (19 mg, 76%). ¹H NMR (CDCl₃): 8.71 (s, 2H), 7.75 (s, 2H), 7.60-7.56 (m, 2H), 7.47-7.39 (m, 4H), 7.15 (d, J=8.7 Hz, 2H), 7.12 (d, J=8.4 Hz, 2H), 3.49 (t, J=7.8 Hz, 1H), 3.34 (d, J=7.8 Hz, 2H), 2.85-2.79 (m, 1H), 1.18 (s, 3H), 1.16 (s, 3H).

### EXAMPLE 8

### N,N′-Bis-(3-trifluoromethyl-phenyl)-2-(4-pyridyl-methylene)-malonamide

The title compound was prepared from a mixture of N,N′-bis-(3-trifluoromethyl-phenyl)-malonamide (50 mg, 0.128 mmol) and 4-pyridinecarboxaldehyde (15 μL, 0.154 mmol) similar to Example 3 and isolated as a white solid (6 mg, 10%). ¹H NMR (CDCl₃): 9.93 (s, 1H), 8.67 (s, 1H), 8.52 (dd, J=1.5, 4.2 Hz, 2H), 7.96 (s, 1H), 7.92 (s, 1H), 7.84 (s, 1H), 7.78-7.74 (m, 1H), 7.53-7.44 (m, 5H), 7.24 (dd, J=1.8, 4.5 Hz, 2H).

### EXAMPLE 9

### 2-(4-Isopropyl-benzylidene)-malonic acid dimethyl ester

To a stirring mixture of dimethyl malonate (87 μL, 0.757 mmol) and 4-isopropylbenzaldehyde (138 μL, 0.908 mmol) in anhydrous THF (5 mL) was added piperidine (75 μL) and glacial acetic acid (43 μL). The mixture was refluxed for 24 h. The solvent was evaporated and the resulting residue was purified by column chromatography to obtain a colorless oil (43 mg, 22%). ¹H NMR (CDCl₃): 7.75 (s, 1H), 7.37 (d, J=8.4 Hz, 2H), 7.24 (d, J=8.1 Hz, 2H), 3.87 (d, J=0.3 Hz, 3H), 3.84 (d, J=0.6 Hz, 3H), 2.97-2.87 (m, 1H), 1.26 (s, 3H), 1.23 (s, 3H).

### EXAMPLE 10

### N,N′-Diphenyl-2-(4-isopropyl-benzylidene)-malonamide

a) N,N′-Diphenyl-malonamide: The title compound was prepared from a mixture of aniline (323 μL, 3.55 mmol) and malonyl dichloride (138 μL, 1.42 mmol) similar to Example 1a and isolated as a tan solid (222 mg, 62%). ¹H NMR (CDCl₃): 8.73 (s, 2H), 7.56 (d, J=7.8 Hz, 4H), 7.36 (t, J=8.0 Hz, 4H), 7.19-7.14 (m, 2H), 3.54 (s, 2H).

b) N,N′-Diphenyl-2-(4-isopropyl-benzylidene)-malonamide: The title compound was prepared from a mixture of N,N′-diphenyl-malonamide (50 mg, 0.197 mmol) and 4-isopropylbenzaldehyde (36 μL, 0.236 mmol) similar to Example 3 and isolated as a yellow solid. ¹H NMR (CDCl₃): 9.81 (s, 1H), 8.07 (s, 1H), 7.73 (s, 1H), 7.63 (d, J=7.2 Hz, 2H), 7.43 (d, J=8.1 Hz, 2H), 7.38-7.32 (m, 5H), 7.21 (d, J=8.4 Hz, 2H), 7.18-7.15 (m, 1H), 7.12 (d, J=7.5 Hz, 2H), 2.91-2.86 (m, 1H), 1.23 (s, 3H), 1.21 (s, 3H).

### EXAMPLE 11

### N,N′-Bis-(2-trifluoromethyl-phenyl)-2-(4-isopropyl-benzylidene)-malonamide

a) N,N′-Bis-(2-trifluoromethyl-phenyl)-malonamide: The title compound was prepared from a mixture of 2-trifluoromethylaniline (446 μL, 3.55 mmol) and malonyl dichloride (138 μL, 1.42 mmol) similar to Example 1a and isolated as a tan solid (329 mg, 59%). ¹H NMR (CDCl₃): 8.79 (s, 2H), 8.12 (d, J=8.4 Hz, 2H), 7.65 (d, J=7.8 Hz, 2H), 7.60 (t, J=7.8 Hz, 2H), 7.30 (t, J=75 Hz, 2H), 3.61 (s, 2H).

b) N,N′-Bis-(2-trifluoromethyl-phenyl)-2-(4-Isopropyl-benzylidene)-malonamide: The title compound was prepared from a mixture of N,N′-bis-(2-trifluoromethyl-phenyl)-malonamide (50 mg, 0.128 mmol) and 4-isopropylbenzaldehyde (23 μL, 0.154 mmol) similar to Example 3 and isolated as a white solid (47 mg, 71%). ¹H NMR (CDCl₃): 9.82 (s, 1H), 8.46 (d, J=8.1 Hz, 1H), 8.32 (d, J=8.1 Hz, 1H), 8.16 (s, 1H), 7.70 (s, 1H), 7.66-7.52 (m, 4H), 7.29-7.24 (m, 2H), 7.46 (d, J=8.4 Hz, 2H), 7.22 (d, J=8.1 Hz, 2H), 2.91-2.84 (m, 1H), 1.22 (s, 3H), 1.19 (s, 3H).

### EXAMPLE 12

### N,N′-Bis-(4-trifluoromethyl-phenyl)-2-(4-isopropyl-benzylidene)-malonamide

a) N,N′-Bis-(4-trifluoromethyl-phenyl)-malonamide: The title compound was prepared from a mixture of 4-aminobenzotrifluoride (446 μL, 3.55 mmol) and malonyl dichloride (138 μL, 1.42 mmol) similar to Example 1a and isolated as a tan solid (358 mg, 65%). ¹H NMR (CDCl₃): 8.93 (s, 2H), 7.70 (d, J=8.7 Hz, 4H), 7.62 (d, J=8.7 Hz, 4H), 3.60 (s, 2H).

b) N,N′-Bis-(4-trifluoromethyl-phenyl)-2-(4-isopropyl-benzylidene)-malonamide: The title compound was prepared from a mixture of N,N′-bis-(4-trifluoromethyl-phenyl)-malonamide (50 mg, 0.128 mmol) and 4-isopropylbenzaldehyde (23 μL, 0.154 mmol) similar to Example 3 and isolated as a white solid (9 mg, 14%). ¹H NMR (CDCl₃): 10.09 (s, 1H), 8.61 (s, 1H), 8.01 (s, 1H), 7.70 (d, J=8.4 Hz, 2H), 7.65-7.50 (m, 4H), 7.56 (d, J=8.7 Hz, 2H), 7.33 (d, J=8.1 Hz, 2H), 7.12 (d, J=7.8 Hz, 2H), 2.85-2.78 (m, 1H), 1.18 (s, 3H), 1.15 (s, 3).

### EXAMPLE 13

### N,N′-Bis-(3-methoxy-phenyl)-2-(4-isopropyl-benzylidene)-malonamide

a) N,N′-Bis-(3-methoxy-phenyl)-malonamide: The title compound was prepared from a mixture of m-anisidine (399 μL, 3.55 mmol) and malonyl dichloride (138 μL, 1.42 mmol) similar to Example 1a and isolated as a white solid (219 mg, 49%). ¹H NMR (CDCl₃): 8.93 (s, 2H), 7.28 (t, J=2.4 Hz, 2H), 7.23 (d, J=8.1 Hz, 2H), 7.10-7.07 (m, 2H), 6.71 (dd, J=2.4, 8.1 Hz, 2H), 3.81 (s, 6H), 3.55 (s, 2H).

b) N,N′-Bis-(3-methoxy-phenyl)-2-(4-isopropyl-benzylidene)-malonamide: The title compound was prepared from a mixture of N,N′-bis-(3-methoxy-phenyl)-malonamide (50 mg, 0.128 mmol) and 4-isopropylbenzaldehyde (29 μL, 0.191 mmol) similar to Example 3 and isolated as a yellow solid (4 mg, 6%). ¹H NMR (CDCl₃): 9.88 (s, 1H), 8.41 (s, 1H), 7.96 (s, 1H), 7.36 (d, J=8.7 Hz, 2H), 7.36-7.35 (m, 1H), 7.24 (d, J=2.7H, 1H), 7.21 (d, J=2.7 Hz, 1H), 7.18 (dd, J=1.8, 3.9 Hz, 1H), 7.12 (d, J=8.4 Hz, 2H), 7.08-7.05 (m, 1H), 6.97-6.94 (m, 1H), 6.75-6.66 (m, 2H), 3.81 (s, 3H), 3.77 (s, 3H), 2.85-2.78 (m, 1H), 1.19 (s, 3H), 1.16 (s, 3H).

### EXAMPLE 14

### N,N′-Bis-(3-chloro-phenyl)-2-(4-isopropyl-benzylidene)-malonamide

a) N,N′-Bis-(3-chloro-phenyl)-malonamide: The title compound was prepared from a mixture of 3-chloroaniline (376 μL, 3.55 mmol) and malonyl dichloride (138 μL, 1.42 mmol) similar to Example 1a and isolated as a tan solid (306 mg, 67%). ¹H NMR (CDCl₃): 9.11 (s, 2H), 7.75 (t, J=2.1 Hz, 2H), 7.40-7.37 (m, 2H), 7.29 (d, J=8.1, 2H), 7.16-7.13 (m, 2H), 3.59 (s, 2H).

b) N,N′-Bis-(3-chloro-phenyl)-2-(4-isopropyl-benzylidene)-malonamide: The title compound was prepared from a mixture of N,N′-bis-(3-chloro-phenyl)-malonamide (50 mg, 0.128 mmol) and 4-isopropylbenzaldehyde (28 μL, 0.186 mmol) similar to Example 3 and isolated as a white solid (22 mg, 31%). ¹H NMR (CDCl₃): 10.03 (s, 1H), 9.31 (s, 1H), 7.83 (s, 1H), 7.73-7.66 (m, 2H), 7.36-7.09 (m, 6H), 7.29 (d, J=8.1 Hz, 2H), 7.04 (d, J=8.1 Hz, 2H), 2.82-2.75 (m, 1H), 1.15 (s, 3H), 1.12 (s, 3H).

### EXAMPLE 15

### N,N′-Bis-(3-trifluoromethyl-phenyl)-2-benzylidene-malonamide

The title compound was prepared from a mixture of N,N′-bis-(3-trifluoromethyl-phenyl)-malonamide (50 mg, 0.128 mmol) and benzaldehyde (14 μL, 0.141 mmol) similar to Example 3 and isolated as a yellow solid (5 mg, 8%). ¹H NMR (CDCl₃): 10.20 (s, 1H), 9.46 (s, 1H), 7.99 (s, 1H), 7.89 (s, 1H), 7.93 (s, 1H), 7.65 (dd, J=2.1, 7.2 Hz, 2H), 7.49-7.43 (m, 4H), 7.33 (d, J=7.5 Hz, 2H), 7.20 (d, J=7.5 Hz, 1H), 7.14 (d, J=7.5 Hz, 2H).

### EXAMPLE 16

### N,N′-Bis-(3-trifluoromethyl-phenyl)-2-(3H-imidazol-4-yl-methylene)-malonamide

The title compound was prepared from a mixture of N,N′-bis-(3-trifluoromethyl-phenyl)-malonamide (50 mg, 0.128 mmol) and 4-formylimidazole (14 mg, 0.141 mmol) similar to Example 3 and isolated as a yellow solid (41 mg, 68%). ¹H NMR (CDCl₃): 11.46 (s, 1H, 10.37 (s, 1H), 8.27 (s, 1H), 8.19 (s, 1H), 7.98-7.96 (m, 1H), 7.88-7.86 (m, 2H), 7.65 (s, 1H), 7.62-7.56 (m, 4H), 7.45-7.43 (m, 2H).

### EXAMPLE 17

### N,N′-Bis-(3-nitro-phenyl)-2-(4-isopropyl-benzylidene)-malonamide

a) N,N′-Bis-(3-nitrophenyl)-malonamide: The title compound was prepared from a mixture of 3-nitroaniline (490 mg, 3.55 mmol) and malonyl dichloride (138 μL, 1.42 mmol) similar to Example 1a and isolated as a yellow solid (252 mg, 52%). ¹H NMR (CDCl₃): 10.73 (s, 2H), 8.66-8.64 (m, 2H), 7.97-7.91 (m, 4H), 7.67-7.61 (m, 2H), 3.59 (s, 2H).

b) N,N′-Bis-(3-nitrophenyl)-2-(4-isopropyl-benzylidene)-malonamide: The title compound was prepared from a mixture of N,N′-bis-(3-nitrophenyl)-malonamide (50 mg, 0.145 mmol) and 4-isopropylbenzaldehyde (24 μL, 0.160 mmol) similar to Example 3 and isolated as a yellow solid (10 mg, 15%). ¹H NMR (CDCl₃): 10.28 (s, 1H), 8.74 (s, 1H), 8.66 (t, J=2.4 Hz, 1H), 8.48 (t, J=2.4 Hz, 1H), 8.11-8.03 (m, 2H), 8.10 (s, 1H), 7.95-7.92 (m, 1H), 7.77-7.74 (m, 1H), 7.61-7.55 (m, 2H), 7.36 (d, J=8.1 Hz, 2H), 7.16 (d, J=8.4 Hz, 2H), 2.88-2.79 (m, 1H), 1.19 (s, 3H), 1.16 (s, 3H).

### EXAMPLE 18

### N,N′-Bis-(3-hydroxy-phenyl)-2-(4-isopropyl-benzylidene)-malonamide

a) N,N′-Bis-(3-hydroxyphenyl)-malonamide: The title compound was prepared from a mixture of 3-aminophenol (387 mg, 3.55 mmol) and malonyl dichloride (138 μL, 1.42 mmol) similar to Example 1a and isolated as an orange solid (376 mg, 92%). ¹H NMR (DMSO-d₆): 10.03 (s, 2H), 9.41 (s, 2H), 7.20-7.18 (m, 2H), 7.11-7.05 (m, 2H), 6.97-6.94 (m, 2H), 6.47-6.44 (m, 2H), 3.42 (s, 2H).

b) N,N′-Bis-(3-hydroxyphenyl)-2-(4-isopropyl-benzylidene)-malonamide: The title compound was prepared from a mixture of N,N′-bis-(3-hydroxy-phenyl)-malonamide (50 mg, 0.145 mmol) and 4-isopropylbenzaldehyde (24 μL, 0.160 mmol) similar to Example 3 and isolated as a tan solid (6 mg, 8%). ¹H NMR (Acetone-d₆): 9.59 (s, 1H), 9.37 (s, 1H), 8.39 (s, 1H), 7.70 (s, 1H), 7.51 (d, J=8.4 Hz, 211H), 7.44-7.42 (m, 1H), 7.36-7.35 (m, 1H), 7.28 (d, J=8.4 Hz, 2H), 7.17-7.07 (m, 3H), 7.03-7.00 (m, 1H), 6.65-6.58 (m, 2H), 2.94-2.88 (m, 1H), 1.22 (s, 3H), 1.20 (s, 3H).

### EXAMPLE 19

### N,N′-Bis-(3-trifluoromethyl-phenyl)-2-[(4-isopropyl-phenyl)-morpholin-4-yl-methyl]-malonamide

To a stirring solution of N,N′-bis-(3-trifluoromethyl-phenyl)-2-(4-isopropyl-benzylidene)-malonamide (25 mg, 0.098 mmol) in anhydrous THF (3 ml) was added morpholine (84 μL, 0.960 mmol). The mixture was stirred at room temperature for 4 h. The solvent was evaporated to dryness and the resulting residue was wash with hexanes to obtain a white solid (28 mg, 96%). ¹H NMR (Acetone-d₆): 10.69 (s, 1H), 9.75 (s, 1H), 8.26 (s, 1H), 7.91-7.88 (m, 2H), 7.63-7.58 (m, 2H), 7.48-7.43 (m, 2H), 7.36-7.33 (m, 1H), 7.30 (d, J=8.1 Hz, 2H), 7.24 (d, J=7.8 Hz, 2H), 4.63 (d, J=12.0 Hz, 1H), 4.48 (d, J=12.0 Hz, 1H), 3.62-3.58 (m, 4H), 2.91-2.84 (m, 1H), 2.64-2.59 (m, 2H), 2.46-2.41 (m, 2H), 1.19 (s, 3H), 1.17 (s, 3H).

### EXAMPLE 20

### N,N′-Bis-(3-bromo-phenyl)-2-(4-isopropyl-benzylidene)-malonamide

a) N,N′-Bis-(3-bromo-phenyl)-malonamide: The title compound was prepared from a mixture of 3-bromoaniline (1.5 mL, 14.2 mmol) and malonyl dichloride (0.691 mL, 7.10 mmol) similar to Example 1a and isolated as a yellow solid (2.81 g, 96%). ¹H NMR (CDCl₃): 8.97 (s, 2H), 7.88 (t, J=1.8 Hz, 2H), 7.46-7.42 (m, 2H), 7.31-7.28 (m, 2H), 7.23 (d, J=7.8 Hz, 2H), 3.56 (s, 2H).

b) N,N′-Bis-(3-bromo-phenyl)-2-(4-isopropyl-benzylidene)-malonamide: The title compound was prepared from a mixture of N,N′-bis-(3bromo-phenyl)-malonamide (100 mg, 0.243 mmol) and 4-isopropylbenzaldehyde (41 μL, 0.267 mmol) similar to Example 3 and isolated as a yellow solid (33 mg, 25%). ¹H NMR (CDCl₃): 10.01 (s, 1H), 9.16 (s, 1H), 7.84 (s, 1H), 7.83-7.82 (m, 2H), 7.44-7.36 (m, 2H), 7.34-7.15 (m, 4H), 7.25 (d, J=7.8 Hz, 2H), 7.05 (d, J=8.1 Hz, 2H), 2.83-2.76 (m, 1H), 1.16 (s, 3H), 1.13 (s, 3H).

### EXAMPLE 21

### N,N′-Bis-(3-trifluoromethyl-phenyl)-2-(6-methyl-pyridin-2-yl-methylene)-malonamide

The title compound was prepared from a mixture of N,N′-bis-(3-trifluoromethyl-phenyl)-malonamide (100 mg, 0.256 mmol) and 6-methyl-pyridine-2-carboxaldehyde (34 mg, 0.282 mmol) similar to Example 3 and isolated as a white solid (55 mg, 44%). ¹H NMR (CDCl₃): 12.45 (s, 1H), 11.16 (s, 1H), 8.13 (s, 1H), 8.03-7.99 (m, 2H), 7.93-7.91 (m, 1H), 7.72-7.69 (m, 1H), 7.58-7.35 (m, 5H), 7.31 (d, J=7.5 Hz, 1H), 7.11 (d, J=7.5 Hz, 1H), 2.40 (s, 3H).

### EXAMPLE 22

### N,N′-Bis-(3-trifluoromethyl-phenyl)-2-(4-ethoxy-benzylidene)-malonamide

The title compound was prepared from a mixture of N,N′-bis-(3-trifluoromethyl-phenyl)-malonamide (100 mg, 0.256 mmol) and 4-ethoxybenzaldehyde (39 μL, 0.282 mmol) similar to Example 3 and isolated as a yellow solid (15 mg, 11%). ¹H NMR (CDCl₃): 10.19 (s, 1H), 9.97 (s, 1H), 8.14 (s, 1H), 7.92 (s, 1H), 7.80-7.79 (m, 1H), 7.77 (s, 1H), 7.61-7.58 (m, 1H), 7.54-7.37 (m, 4H), 7.25 (d, J=8.7 Hz, 2H), 6.55 (d, J=9.0 Hz, 2H), 3.77 (q, J=6.9 Hz, 2H), 1.33 (t, J=6.9 Hz, 3H).

### EXAMPLE 23

### N,N′-Bis-(3-trifluoromethyl-phenyl)-2-(5-methyl-furan-2-yl-methylene)-malonamide

The title compound was prepared from a mixture of N,N′-bis-(3-trifluoromethyl-phenyl)-malonamide (100 mg, 0.256 mmol) and 5-methylfurfural (28 μL, 0.282 mmol) similar to Example 3 and isolated as an orange solid (67 mg, 45%). ¹H NMR (CDCl₃): 10.04 (s, 1H), 9.32 (s, 1H), 8.13 (s, 1H), 7.97-7.94 (m, 2H), 7.65-7.34 (m, 5H), 7.61 (s, 1H), 6.71 (d, J=3.3 Hz, 1H), 5.97 (dd, J=0.9, 3.3 Hz, 1H), 2.04 (s, 3H).

### EXAMPLE 24

### N,N′-Bis-(3-methoxy-phenyl)-2-(4-chloro-2-nitro-benzylidene)-malonamide

The title compound was prepared from a mixture of N,N′-bis-(3-methoxy-phenyl)-malonamide (50 mg, 0.159 mmol) and 4-chloro-2-nitro-benzaldehyde (32 mg, 0.175 mmol) similar to Example 13b and isolated as a yellow solid (5 mg, 7%). ¹H NMR (CDCl₃): 9.32 (s, 1H), 8.97 (s, 1H), 8.00 (s, 2H), 7.56 (dd, J=2.1, 8.4 Hz, 1H), 7.45 (d, J=8.7 Hz, 1H), 7.36 (t, J=2.1 Hz, 1H), 7.28-7.18 (m, 2H), 7.13-7.10 (m, 1H), 7.07 (t, J=2.1 Hz, 1H), 6.95-6.91 (m, 1H), 6.75-6.68 (m, 2H), 3.81 (s, 3H), 3.78 (s, 3H).

### EXAMPLE 25

### N,N′-Bis-(pyridin-3-yl)-2-(4-isopropyl-benzylidene)-malonamide

The title compound was prepared from a mixture of N,N′-bis-(pyridin-3-yl)-malonamide (25 mg, 0.098 mmol) and 4-isopropylbenzaldehyde (16 μL, 0.108 mmol) similar to Example 3 and isolated as a white solid (7 mg, 18%). ¹H NMR (CDCl₃): 9.95 (s, 1H), 8.75 (s, 1H), 8.44-8.38 (m, 3H), 8.18-8.14 (m, 1H), 8.11 (s, 1H), 8.04-8.00 (m, 2H), 7.39 (d, J=8.1 Hz, 2H), 7.35-7.30 (m, 2H), 7.21 (d, J=8.1 Hz, 2H), 2.93-2.85 (m, 1H), 1.23 (s, 3H), 1.20 (s, 3H).

### EXAMPLE 26

### N,N′-Bis-(3-trifluoromethyl-phenyl)-2-(4-ethyl-benzylidene)-malonamide

The title compound was prepared from a mixture of N,N′-bis-(3-trifluoromethyl-phenyl)-malonamide (100 mg, 0.256 mmol) and 4-ethylbenzaldehyde (37 mg, 0.282 mmol) similar to Example 3 and isolated as a white solid (17 mg, 13%). ¹H NMR (CDCl₃): 10.17 (s, 1H). 9.15 (s, 1H), 7.95 (s, 1H), 7.92 (s, 1H), 7.88 (s, 1H), 7.71-7.66 (m, 2H), 7.52-7.39 (m, 4H), 7.27 (d, J=7.5 Hz, 2H), 7.01 (d, J=7.8 Hz, 2H), 2.51 (q, J=7.8 Hz, 2H), 1.11 (t, J=7.5 Hz, 3H).

### EXAMPLE 27

### N,N′-Bis-(3-trifluoromethyl-phenyl)-2-(4-methylsulfonyl-benzylidene)-malonamide

The title compound was prepared from a mixture of N,N′-bis-(3-trifluoromethyl-phenyl)-malonamide (100 mg, 0.256 mmol) and 4-methylsulfonylbenzaldehyde (52 mg, 0.282 mmol) similar to Example 3 and isolated as a white solid (4 mg, 3%). ¹H NMR (CDCl₃): 9.89 (s, 1H), 8.66 (s, 1H), 7.99 (s, 1H), 7.93 (s, 1H), 7.86 (s, 1H), 7.82 (d, J=8.4 Hz, 2H), 7.77-7.75 (m, 1H), 7.58 (d, J=8.1 Hz, 2H), 7.55-7.45 (m, 5H), 2.95 (s, 3).

### EXAMPLE 28

### N,N′-Bis-(3-trifluoromethyl-phenyl)-2-(4-cyano-benzylidene)-malonamide

The title compound was prepared from a mixture of N,N′-bis-(3-trifluoromethyl-phenyl)-malonamide (50 mg, 0.128 mmol) and 4-cyanobenzaldehyde (18 mg, 0.141 mmol) similar to Example 3 and isolated as a white solid (6 mg, 9%). ¹H NMR (CDCl₃): 10.01 (s, 1H), 9.28 (s, 1H), 7.99 (s, 1H), 7.96 (s, 1H), 7.80 (s, 1H), 7.70-7.67 (m, 1H), 7.62-7.58 (m, 1H), 7.55-7.50 (m, 41H), 7.43 (s, 4H).

### EXAMPLE 29

### N,N′-Bis-(3-trifluoromethyl-phenyl)-2-(2-chloro-pyridin-3-yl-methylene)-malonamide

The title compound was prepared from a mixture of N,N′-bis-(3-trifluoromethyl-phenyl)-malonamide (50 mg, 0.128 mmol) and 2-chloronicotinaldehyde (20 mg, 0.141 mmol) similar to Example 3 and isolated as a yellow solid (5 mg, 8%). ¹H NMR (CDCl₃): 10.23 (s, 1H), 10.20 (s, 1H), 8.14 (dd, J=1.5, 4.5 Hz, 1H), 8.03 (s, 1H), 7.91 (s, 1H), 7.85 (s, 1H), 7.76-7.70 (m, 3H), 7.55-7.45 (m, 4H), 7.12 (dd, J=4.5, 7.5 Hz, 1H).

### EXAMPLE 30

### N,N′-Bis-(3-trifluoromethyl-phenyl)-2-(6-chloro-benzo[1,3]dioxol-5-yl-methylene)-malonamide

The title compound was prepared from a mixture of N,N′-bis-(3-trifluoromethyl-phenyl)-malonamide (50 mg, 0.256 mmol) and 6-chloropiperonal (52 mg, 0.282 mmol) similar to Example 3 and isolated as a yellow solid (8 mg, 6%). ¹H NMR (DMSO-d₆): 10.93 (s, 1H), 10.50 (s, 1H), 8.16 (s, 1H), 8.09 (s, 1H), 7.99-7.97 (m, 1H), 7.83-7.79 (m, 1H), 7.74 (s, 1), 7.63-7.57 (m, 2H), 7.49-7.47 (m, 2H), 7.28 (s, 1H), 7.01 (s, 1H), 6.09 (s, 2H).

### EXAMPLE 31

### 3-(4-Isopropyl-phenyl)-2-(3-trifluoromethyl-phenylcarbamoyl)-acrylic acid methyl ester

a) N-(3-Trifluoromethyl-phenyl)-malonamic acid methyl ester: To a stirring solution of 3-trifluoromethyl aniline (4.6 mL, 36.5 mmol) in anhydrous dichloromethane (50 mL) was slowly added methyl malonyl chloride (4.3 mL, 33.2 mmol). The mixture was stirred at room temperature for 5 minutes. Then the solvent was evaporated to dryness to obtain a white solid (8.6 g, 99%). ¹H NMR (CDCl₃): 9.43 (s, 1H), 7.86 (s, 1H), 7.79-7.76 (m, 1H), 7.48-7.37 (m, 2H), 3.83 (s, 3H), 3.52 (s, 2H).

b) 3-(4-Isopropyl-phenyl)-2-(3-trifluoromethyl-phenylcarbamoyl)-acrylic acid methyl ester: The title compound was prepared from a mixture of N-(3-trifluoromethyl-phenyl)-malonamic acid methyl ester (100 mg, 0.383 mmol) and 4-isopropylbenzaldehyde (64 μL, 0.421 mmol) similar to Example 3 and isolated as a white solid (72 mg, 48%). ¹H NMR (CDCl₃): 8.35 (s, 1H), 7.85 (s, 1H), 7.80-7.77 (m, 1H), 7.72 (s, 1H), 7.45-7.37 (m, 2H), 7.43 (d, J=8.4 Hz, 2H), 7.18 (d, J=8.7 Hz, 2H), 3.77 (s, 3H), 2.90-2.84 (m, 1H), 1.21 (s, 3H), 1.19 (s, 3H).

### EXAMPLE 32

### N,N′-Bis-(quinolin-6-yl)-2-(4-isopropyl-benzylidene)-malonamide

a) N,N′-Bis-(quinolin-6-yl)-malonamide: To a stirring solution of 6-aminoquinoline (169 mg, 1.17 mmol) in anhydrous dichloromethane (20 mL) was slowly added malonyl dichloride (104 μL, 1.06 mmol). The mixture was stirred at room temperature for 5 minutes. Then the solvent was evaporated and the resulting residue was diluted with water. The pH of aqueous solution was adjusted to neutral pH with 2N NaOH. The solid was filtered and washed with excess water to obtain a gray solid (269 mg, 71%). ¹H NMR (DMSO-d₆): 10.58 (s, 2H), 8.81-8.79 (m, 2H), 8.42 (d, J=2.1 Hz, 2H), 8.31 (d, J=8.1 Hz, 2H), 7.99 (d, J=9.0 Hz, 2H), 7.82 (dd, J=2.1, 9.0 Hz, 2H), 7.49 (dd, J=4.2, 8.1 Hz, 2H), 3.65 (s, 2H).

b) N,N′-Bis-(quinolin-6-yl)-2-(4-isopropyl-benzylidene)-malonamide: The title compound was prepared from a mixture of N,N′-di-quinolin-6-yl-malonamide (50 mg, 0.140 mmol) and 4-isopropylbenzaldehyde (23.4 μL) similar to Example 3 and isolated as a white solid (8 mg). ¹H NMR (CDCl₃): 10.18 (s, 1H), 8.90-8.85 (m, 2H), 8.41 (d, J=2.1 Hz, 1H), 8.31 (d, J=2.4 Hz, 1H), 8.28 (s, 1H), 8.19-8.03 (m, 4H), 8.17 (s, 1H), 7.77 (dd, J=2.4, 9.0 Hz, 1H), 7.47-7.37 (m, 3H), 7.44 (d, J=8.1 Hz, 2H), 7.17 (d, J=8.1 Hz, 2H), 2.86-2.77 (m, 1H), 1.16 (s, 3H), 1.14 (s, 3H).

### EXAMPLE 33

### (E) and (Z)-2-(4-Isopropyl-benzylidene)-N-phenyl-N′-(3-trifluoromethyl-phenyl)-malonamide

a) N-(3-Trifluoromethyl-phenyl)-malonamic acid methyl ester: To a stirring solution of 3-trifluoromethyl aniline (4.6 mL, 36.5 mmol) in anhydrous dichloromethane (50 mL) was slowly added methyl malonyl chloride (4.3 mL, 33.2 mmol). The mixture was stirred at room temperature for 5 minutes. Then the solvent was evaporated to obtain a white solid (8.4 g, 97%). ¹H NMR (CDCl₃): 9.43 (s, 1H), 7.86 (s, 1H), 7.77 (d, J=7.8 Hz, 1H), 7.46 (t, J=7.8 Hz, 1H), 7.38 (d, J=7.5 Hz, 1H), 3.83 (s, 3H), 3.52 (s, 2H).

b) N-(3-Trifluoromethyl-phenyl)-malonamic acid: A solution of N-(3-trifluoromethyl-phenyl)-malonamic acid methyl ester (7 g, 26.8 mmol) in aqueous 2N NaOH was stirred at room temperature for 2 h. Then the mixture was acidified with concentrated HCl and extracted several times with ethyl acetate to obtain a yellow solid (6.3 g, 96%). ¹H NMR (CDCl₃): 8.96 (s, 1H), 7.84 (s, 1H), 7.75-7.73 (m, 1H), 7.50-7.41 (m, 2H), 3.58 (s, 2H).

c) N-Phenyl-N′-(3-trifluoromethyl-phenyl)-malonamide: To a stirring solution of N-(3-trifluoromethyl-phenyl)-malonamic acid (45 mg, 0.182 mmol) in anhydrous THF (5 mL) was added 1-(3-dimethylaminopropyl)-3ethyl-carbodiimide hydrochloride (35 mg, 0.182 mmol). The mixture was stirred at room temperature for 15 minutes. Then aniline (16.6 μL, 0.182 mmol) was added and the mixture was stirred at room temperature for 4 h. The solvent was evaporated and the residue was dissolved in aqueous 1N NaOH. The resulting solid was filtered and wash with excess water to obtain a white solid (17 mg, 29%). ¹H NMR (CDCl₃): 9.77 (s, 1H), 9.14 (s, 1H), 7.98 (s, 1H), 7.75-7.71 (m, 1H), 7.59-7.56 (m, 2H), 7.47-7.33 (m, 4H), 7.20-7.14 (m, 1H), 3.65 (s, 2H).

d) (E) and (Z)-2-(4-Isopropyl-benzylidene)-N-phenyl-N′-(3-trifluoromethyl-phenyl)-malonamide: The title compound was prepared from a mixture of N-phenyl-N′-(3-trifluoromethyl-phenyl)-malonamide (17 mg, 0.053 mmol) and 4-isopropylbenzaldehyde (8.8 μL, 0.058 mmol) similar to Example 3 and isolated as yellow solids. Compound A (1 mg, 4%). ¹H NMR (CDCl₃): 10.11 (s, 1H), 8.09 (s, 1H), 7.98 (s, 1H), 7.82-7.78 (m, 2H), 7.66-7.56 (m, 1H), 7.49-7.35 (m, 6H), 7.34 (s, 1H), 7.22-7.14 (m, 3H), 2.91-2.84 (m, 1H), 1.23 (s, 3H), 1.20 (s, 3H). Compound B (1 mg, 4%). ¹H NMR (CDCl₃): 9.77 (s, 1H), 8.12 (s, 1H), 7.77 (s, 1H), 7.66 (d, J=7.2 Hz, 2H), 7.58 (s, 1H), 7.54-7.48 (m, 1H), 7.46-7.34 (m, 6H), 7.22 (d, J=7.8 Hz, 2H), 7.18-7.13 (m, 1H), 2.94-2.85 (m, 1H), 1.23 (s, 3H), 1.21 (s, 3H).

### EXAMPLE 34

### (E) and (Z)-2-(4-Isopropyl-benzylidene)-N-(3-methoxy-phenyl)-N′-(3-trifluoromethyl-phenyl)-malonamide

a) N-(3-Methoxy-phenyl)-N′-(3-trifluoromethyl-phenyl)-malonamide: The title compound was prepared from a mixture of N-(3-trifluoromethyl-phenyl)-malonamic acid (122 mg, 0.494 mmol) and m-anisidine (56 μL, 0.494 mmol) similar to Example 33c and isolated as a white solid (60 mg, 35%). ¹H NMR (CDCl₃): 9.34 (s, 1H), 8.44 (s, 1H), 7.93 (s, 1H), 7.76-7.73 (m, 1H), 7.49-7.39 (m, 2H), 7.29-7.23 (m, 2H), 7.07-7.04 (m, 1H), 6.75-6.71 (m, 1H), 3.82 (s, 3H), 3.55 (s, 2H).

b) (E) and (Z)-2-(4-Isopropyl-benzylidene)-N-(3-methoxy-phenyl)-N′-(3-trifluoromethyl-phenyl)-malonamide: The title compound was prepared from a mixture of N-(3-methoxy-phenyl)-N′-(3-trifluoromethyl-phenyl)-malonamide (30 mg, 0.085 mmol) and 4-isopropylbenzaldehyde (14 μL, 0.094 mmol) similar to Example 3 and isolated as yellow solids. Compound A (1 mg, 2%). ¹H NMR (CDCl₃): 10.06 (s, 1H), 8.11 (s, 1H), 7.99 (s, 1H), 7.84-7.81 (m, 1H), 7.55 (s, 1H), 7.49-7.37 (m, 3H), 7.43 (d, J=8.1 Hz, 2H), 7.23 (d, J=8.1 Hz, 2H), 7.07-7.06 (m, 1H), 6.79-6.71 (m, 2H), 3.81 (s, 3H), 2.93-2.87 (m, 1H), 1.23 (s, 3H), 1.21 (s, 3H). Compound B (1 mg, 2%). ¹H NMR (CDCl₃): 9.81 (s, 1H), 8.12 (s, 1H), 8.07 (s, 1H), 7.63 (s, 1H), 7.58-7.55 (m, 1H), 7.48-7.35 (m, 5H), 7.25-7.08 (m, 4H), 6.72-6.69 (m, 1H), 3.81 (s, 3H), 2.91-2.83 (m, 1H), 1.20 (s, 3H), 1.18 (s, 3H).

### EXAMPLE 35

### (E) and (Z)-N-(6-Bromo-pyridin-2-yl)-2-(4-isopropyl-benzylidene)-N′-(3-trifluoromethyl-phenyl)-malonamide

a) N-(6-Bromo-pyridin-2-yl)-N′-(3-trifluoromethyl-phenyl)-malonamide: The title compound was prepared from a mixture of N-(3-trifluoromethyl-phenyl)-malonamic acid (200 mg, 0.809 mmol) and 2-amino-6-bromo-pyridine (140 mg, 0.809 mmol) similar to Example 33c and isolated as a tan solid (85 mg, 26%). The crude product was used in the next step without purification.

b) (E) and (Z)-N-(6-Bromo-pyridin-2-yl)-2-(4-isopropyl-benzylidene)-N′-(3-trifluoromethyl-phenyl)-malonamide: The title compound was prepared from a mixture of N-(6-bromo-pyridin-2-yl)-N′-(3-trifluoromethyl-phenyl)-malonamide (85 mg, 0.211 mmol) and 4-isopropylbenzaldehyde (38 μL, 0.254 mmol) similar to Example 3 and isolated as white solids. Compound A (1 mg, 1%). ¹H NMR (CDCl₃): 9.68 (s, 1H), 8.28 (d, J=8.4 Hz, 1H), 8.18 (s, 1H), 8.02 (s, 1H), 7.93 (s, 1H), 7.86 (d, J=8.1 Hz, 1H), 7.65 (t, J=8.1 Hz, 1H), 7.51-7.45 (m, 2H), 7.41 (d, J=8.1 Hz, 2H), 7.29-7.27 (m, 1H), 7.23 (d, J=8.1 Hz, 2H), 2.95-2.86 (m, 1H), 1.25 (s, 3H), 1.22 (s, 3H). Compound B (1 mg, 1%). ¹H NMR (CDCl₃): 10.07 (s, 1H), 8.22 (d, J=8.4 Hz, 1H), 8.12 (s, 1H), 7.66-7.61 (m, 1H), 7.63 (s, 1H), 7.57 (t, J=8.4 Hz, 1H), 7.51-7.46 (m, 2H), 7.45-7.44 (m, 2H), 7.42 (d, J=8.1 Hz, 2H), 7.24 (d, J=8.1 Hz, 2H), 2.93-2.88 (m, 1H), 1.24 (s, 3H), 1.22 (s, 3H).

### EXAMPLE 36

### N,N′-Bis-(3-trifluoromethyl-phenyl)-2-(4-isopropoxy-benzylidene)-malonamide

The title compound was prepared from a mixture of N,N′-bis-(3-trifluoromethyl-phenyl)-malonamide (100 mg, 0.256 mmol) and 4-isopropoxybenzaldehyde (46 mg, 0.282 mmol) similar to Example 3 and isolated as a white solid (13 mg, 9%). ¹H NMR (CDCl₃): 10.06 (s, 1H), 8.83 (s, 1H), 7.95 (s, 2H), 7.92 (s, 1H), 7.74-7.72 (m, 1H), 7.67-7.64 (m, 1H), 7.52-7.38 (m, 4H), 7.35 (d, J=8.4 Hz, 2H), 6.71 (d, J=8.7 Hz, 2H), 4.46-4.40 (m, 1H), 1.29 (s, 3H), 1.27 (s, 3H).

### EXAMPLE 37

### N,N′-Bis-(3-trifluoromethyl-phenyl)-2-(2-chloro-4-hydroxy-benzylidene)-malonamide

The title compound was prepared from a mixture of N,N′-bis-(3-trifluoromethyl-phenyl)-malonamide (100 mg, 0.256 mmol) and 2-chloro-4-hydroxybenzaldehyde (44 mg, 0.282 mmol) similar to Example 3 and isolated as a white solid (3 mg, 2%). ¹H NMR (Acetone-d₆): 10.09 (s, 1H), 9.81 (s, 1H), 9.37 (s, 1H), 8.27 (s, 1H), 8.13 (s, 1H), 8.08 (s, 1H), 7.95-7.92 (m, 1H), 7.78-7.75 (m, 1H), 7.62-7.45 (m, 5H), 7.01 (d, J=2.4 Hz, 1H), 6.79 (dd, J=2.4, 8.7 Hz, 1H).

### EXAMPLE 38

### N,N′-Bis-(3-trifluoromethyl-phenyl)-2-(2,3-dihydro-benzofuran-5-yl-methylene)-malonamide

The title compound was prepared from a mixture of N,N′-bis-(3-trifluoromethyl-phenyl)-malonamide (50 mg, 0.128 mmol) and 2,3-dihydrobenzo[B]furan-5-carboxaldehyde (21 mg, 0.141 mmol) similar to Example 3 and isolated as a yellow solid (12 mg, 18%). ¹H NMR (CDCl₃): 10.06 (s, 1H), 8.85 (s, 1H), 7.97 (s, 1H), 7.95 (s, 1H), 7.91 (s, 1H), 7.72 (d, J=8.1 Hz, 1H), 7.67-7.64 (m, 1H), 7.54-7.39 (m, 4H), 7.23-7.21 (m, 2H), 6.63 (d, J=8.1 Hz, 1H), 4.51 (t, J=8.7 Hz, 2H), 2.93 (t, J=8.7 Hz, 2H).

### EXAMPLE 39

### N,N′-Bis-(3-trifluoromethyl-phenyl)-2-(2,3-dihydro-benzo[1,4]dioxin-6-yl-methylene)-malonamide

The title compound was prepared from a mixture of N,N′-bis-(3-trifluoromethyl-phenyl)-malonamide (100 mg, 0.256 mmol) and 1,4-benzodioxan-6-carboxaldehyde (50 mg, 0.307 mmol) similar to Example 3 and isolated as a white solid (43 mg, 31%). ¹H NMR (CDCl₃): 10.12 (s, 1H), 9.09 (s, 1H), 7.96 (s, 1H), 7.89 (s, 1H), 7.83 (s, 1H), 7.75-7.72 (m, 2H), 7.54-7.38 (m, 4H), 6.92-6.88 (m, 2H), 6.68 (d, J=9.0 Hz, 1H), 4.15-4.11 (m, 2H), 4.06-4.03 (m, 2H).

### EXAMPLE 40

### N,N′-Bis-(3-trifluoromethyl-phenyl)2-(4-pyrrolidin-1-yl-benzylidene)-malonamide

The title compound was prepared from a mixture of N,N′-bis-(3-trifluoromethyl-phenyl)-malonamide (50 mg, 0.128 mmol) and 4-(1-pyrrolidino)benzaldehyde (27 mg, 0.154 mmol) similar to Example 3 and isolated as a yellow solid (4 mg, 6%). ¹H NMR (CDCl₃): 10.16 (s, 1H), 9.72 (s, 1H), 9.43 (s, 1H), 8.07 (s, 1H), 7.95 (s, 1H), 7.91 (s, 1H), 7.84-7.82 (m, 1H), 7.73 (d, J=9.0 Hz, 2H), 7.64-7.62 (m,1H), 7.54-7.30 (m, 3H), 6.57 (d, J=9.0 Hz, 1H), 6.25 (d, J=9.0 Hz, 1H), 3.42-3.37 (m, 2H), 3.17-3.13 (m, 2H), 2.08-2.03 (m, 2H), 2.00-1.95 (m, 2H).

### EXAMPLE 41

### N,N′-Bis-(3-trifluoromethyl-phenyl)-2-(3,4-dichloro-benzylidene)-malonamide

The title compound was prepared from a mixture of N,N′-bis-(3-trifluoromethyl-phenyl)-malonamide (50 mg, 0.128 mmol) and 3,4-dichlorobenzaldehyde (27 mg, 0.154 mmol) similar to Example 3 and isolated as a white solid (7 mg, 10%). ¹H NMR (CDCl₃): 9.98 (s, 1H), 9.07 (s, 1H), 7.97 (s, 1H), 7.94 (s, 1H), 7.75 (s, 1H), 7.74-7.71 (m, 1H), 7.64-7.62 (m, 1H), 7.56-7.46 (m, 4H), 7.43 (d, J=2.1 Hz, 1H), 7.28 (d, J=8.4 Hz, 1H), 7.21 (dd, J=2.1, 8.4 Hz, 1H).

### EXAMPLE 42

### N,N′-Bis-(5-chloro-2-hydroxy-phenyl)-2-(4-isopropyl-benzylidene)-malonamide

a) N,N′-Bis-(5-chloro-2-hydroxy-phenyl)-malonamide: To a stirring solution of 2-amino-4-chlorophenol (500 mg, 3.48 mmol) in 1,4-dioxane (10 mL) was slowly added malonyl dichloride (339 μL, 3.48 mmol). The mixture was stirred at room temperature for 15 minutes and the resulting solid was collected by filtration to obtain a gray solid (289 mg, 23%). The intermediate was used in the next step without purification.

b) N,N′-Bis-(5-chloro-2-hydroxy-phenyl)-2-(4-isopropyl-benzylidene)-malonamide: The title compound was prepared from a mixture of N,N′-bis-(5-chloro-2-hydroxy-phenyl)-malonamide (50 mg, 0.141 mmol) and 4-isopropylbenzaldehyde (24 μL, 0.155 mmol) similar to Example 3 and isolated as an orange solid (5 mg, 7%). ¹H NMR (Acetone-d₆): 9.79 (s, 1H), 9.23 (s, 1H), 8.42 (d, J=2.4 Hz, 1H), 8.02 (d, J=2.4 Hz, 1H), 7.94 (s, 1H), 7.55 (d, J=8.1 Hz, 2H), 7.32 (d, J=8.1 Hz, 2H), 7.05 (dd, J=2.7, 8.7 Hz, 1H), 6.98-6.97 (m, 2H), 6.91 (d, J=8.7 Hz, 1H), 2.90-2.88 (m, 1H), 1.24 (s, 3H), 1.21 (s, 3H).

### EXAMPLE 43

### N,N′-Bis-(3-trifluoromethyl-phenyl)-2-(2-chloro-4-dimethylamino-benzylidene)-malonamide

The title compound was prepared from a mixture of N,N′-bis-(3-trifluoromethyl-phenyl)-malonamide (50 mg, 0.128 mmol) and 2-chloro-4-(dimethylamino)benzaldehyde (26 mg, 0.141 mmol) similar to Example 3 and isolated as a yellow solid (4 mg, 6%). ¹H NMR (CDCl₃): 10.19 (s, 1H), 9.53 (s, 1H), 8.26 (s, 1H), 8.05 (s, 1H), 7.97 (s, 1H), 7.82-7.79 (m, 1H), 7.69-7.66 (m, 1H), 7.53-7.41 (m, 3H), 7.37-7.34 (m, 1H), 7.35 (d, J=8.7 Hz, 1H), 6.40 (dd, J=2.4, 9.0 Hz, 1H), 6.31 (d, J=2.7 Hz, 1H), 2.89 (s, 6H).

### EXAMPLE 44

### N,N′-Bis-(3-trifluoromethyl-phenyl)-2-(2,5-dichloro-benzylidene)-malonamide

The title compound was prepared from a mixture of N,N′-bis-(3-trifluoromethyl-phenyl)-malonamide (50 mg, 0.128 mmol) and 2,5-dichlorobenzaldehyde (49 mg, 0.141 mmol) similar to Example 3 and isolated as a white solid (7 mg, 100%). ¹H NMR (Acetone-d₆): 10.17 (s, 1H), 9.87 (s, 1H), 8.26 (s, 1H), 8.07 (s, 1H), 8.02 (s, 1H), 7.97-7.95 (m, 1H), 7.75-7.73 (m, 1H), 7.64-7.62 (m, 3H), 7.58 (d, J=8.4 Hz, 1H), 7.51-7.48 (m, 2H), 7.45 (dd, J=2.4, 8.4 Hz, 1H).

### EXAMPLE 45

### N,N′-Bis-(3-trifluoromethyl-phenyl)-2-[3,5-dichloro-2-(2-morpholin-4-yl-ethoxy)-benzylidene]-malonamide

a) 3,5-Dichloro-2-(2-morpholin-4-yl-ethoxy)-benzaldehyde: A mixture of 3,5-dichlorosalicylaldehyde (200 mg, 1.05 mmol), N-(2-chloroethyl)morpholine hydrochloride (214 mg, 1.15 mmol), and sodium carbonate (122 mg, 1.15 mmol) in aqueous ethanol (8 mL) was refluxed for 20 h. The mixture was diluted with ethyl acetate (100 mL), washed with water (1×50 mL), brine (1×50 mL) and dried over anhydrous sodium sulfate. The solvent was evaporated and the resulting residue was column purified to obtain a tan oil (210 mg, 66%). ¹H NMR (CDCl₃): 10.42 (s, 1H), 7.71 (d, J=2.7 Hz, 1H), 7.60 (d, J=2.4 Hz, 1H), 4.27 (t, J=5.7 Hz, 2H), 3.66 (t, J=4.5 Hz, 4H), 2.76 (t, J=5.7 Hz, 2H), 2.48 (t, J=4.5 Hz, 4H).

b) N,N′-Bis-(3-trifluoromethyl-phenyl)-2-[3,5-dichloro-2-(2-morpholin-4-yl-ethoxy)-benzylidene]-malonamide: The title compound was prepared from a mixture of N,N′-bis-(3-trifluoromethyl-phenyl)-malonamide (567 mg, 1.45 mmol) and 3,5-dichloro-2-(2-morpholin-4-yl-ethoxy)-benzaldehyde (400 mg, 1.32 mmol) similar to Example 3 and isolated as a white solid (151 mg, 15%). ¹H NMR (CDCl₃): 10.05 (s, 1H), 9.77 (s, 1H), 8.02 (s, 1H), 7.96 (s, 1H), 7.83 (s, 1H), 7.81-7.73 (m, 2H), 7.52-7.41 (m, 4H), 7.34 (d, J=2.4 Hz, 1H), 7.16 (dd, J=0.6, 2.4 Hz, 1H), 4.11 (t, J=4.8 Hz, 2H), 3.52 (t, J=4.5 Hz, 4H), 2.78 (t, J=4.8 Hz, 2H), 2.49 (t, J=4.5 Hz, 4H).

### EXAMPLE 46

### N,N′-Di-(pyridin-2-yl)-2-(4-isopropyl-benzylidene)-malonamide

The title compound was prepared from a mixture of N,N′-di-pyridin-2-yl)-malonamide (25 mg, 0.098 mmol) and 4-isopropylbenzaldehyde (16 μL, 0.107 mmol) similar to Example 3 and isolated as a yellow solid (1 mg, 3%). ¹H NMR (CDCl₃): 9.90 (s, 1H), 8.39-8.29 (m, 2H), 8.20-8.18 (m, 1H), 8.11 (s, 1H), 8.09 (s, 1H), 7.79-7.71 (m, 2H), 7.45 (d, J=8.1 Hz, 2H), 7.30-7.22 (m, 1H), 7.20 (d, J=7.8 Hz, 2H), 7.12-7.01 (m, 2H), 2.92-2.85 (m, 1H), 1.22 (s, 3H), 1.20 (s, 3H).

### EXAMPLE 47

### N,N′-Bis-(3-trifluoromethyl-phenyl)-2-(5-methyl-pyridin-3-yl-methylene)-malonamide

The title compound was prepared from a mixture of N,N′-bis-(3-trifluoromethyl-phenyl)-malonamide (50 mg, 0.128 mmol) and 5-methyl-pyridine-3-carbaldehyde (17 mg, 0.141 mmol) similar to Example 3 and isolated as a white solid (8 mg, 13%). ¹H NMR (CDCl₃): 10.05 (s, 1H), 10.01 (s, 1H), 8.28 (d, J=1.5 Hz, 1H), 8.14 (d, J=1.8 Hz, 1H), 8.01 (s, 2H), 7.75 (s, 1H), 7.74-7.71 (m, 2H), 7.58-7.45 (m, 5H), 2.11 (s, 3H).

### EXAMPLE 48

### N,N′-Bis-(3-trifluoromethyl-phenyl)-2-[5-chloro-2-(2-morpholin-4-yl-ethoxy)-benzylidene]-malonamide

a) 5-Chloro-2-(2-morpholin-4-yl-ethoxy)-benzaldehyde: The title compound was prepared from a mixture of 5-chlorosalicylaldehyde (400 mg, 2.55 mmol) and N-(2-chloroethyl)morpholine hydrochloride (523 mg, 2.81 mmol) similar to Example 45a and isolated a brown oil (345 mg, 50%). ¹H NMR (CDCl₃): 10.42 (s, 1H), 7.79 (d, J=2.7 Hz, 1H), 7.49 (dd, J=2.7, 8.7 Hz, 1H), 6.95 (d, J=9.0 Hz, 1H), 4.22 (t, J=6.0 Hz, 2H), 3.72 (t, J=4.5 Hz, 4H), 2.86 (t, J=6.0 Hz, 2H), 2.58 (t, J=4.5 Hz, 4H).

b) N,N′-Bis-(3-trifluoromethyl-phenyl)-2-[5-chloro-2-(2-morpholin-4-yl-ethoxy)-benzylidene]-malonamide: The title compound was prepared from a mixture of N,N′-bis-(3-trifluoromethyl-phenyl)-malonamide (50 mg, 0.128 mmol) and 5-chloro-2-(2-morpholin-4-yl-ethoxy)-benzaldehyde (35 mg, 0.128 mmol) similar to Example 3 and isolated as a white solid (2 mg, 2%). ¹H NMR (CDCl₃): 10.42 (s, 1H), 10.31 (s, 1H), 7.85 (s, 1H), 7.71 (s, 1H), 7.68 (s, 1H), 7.58-7.54 (m, 2H), 7.51-7.41 (m, 4H), 7.20 (dd, J=2.4, 9.0 Hz, 1H), 7.02 (d, J=2.4 Hz, 1H), 6.85 (d, J=9.0 Hz, 1H), 4.22 (s, 2H), 3.98 (s, 2H), 3.32 (s, 3H), 2.28 (s, 3H), 2.22 (s, 2H).

### EXAMPLE 49

### N,N′-Bis-(3-trifluoromethyl-phenyl)-2-(3,5-dimethoxy-benzylidene)-malonamide

The title compound was prepared from a mixture of N,N′-bis-(3-trifluoromethyl-phenyl)-malonamide (100 mg, 0.256 mmol) and 3,5-dimethoxybenzaldehyde (47 mg, 0.282 mmol) similar to Example 3 and isolated as a white solid (39 mg, 28%). ¹H NMR (CDCl₃): 10.10 (s, 1H), 8.86 (s, 1H), 7.94 (s, 1H), 7.91 (s, 1H), 7.88 (s, 1H), 7.74-7.72 (m, 1H), 7.66-7.62 (m, 1H), 7.52-7.39 (m, 4H), 6.50 (d, J=2.1 Hz, 2H), 6.36 (t, J=2.1 Hz, 1H), 3.53 (s, 6H).

### EXAMPLE 50

### N,N′-Bis-(3-trifluoromethyl-phenyl)-2-(3,4,5-trimethoxy-benzylidene)-malonamide

The title compound was prepared from a mixture of N,N′-bis-(3-trifluoromethyl-phenyl)-malonamide (100 mg, 0.256 mmol) and 3,4,5-trimethoxybenzaldehyde (55 mg, 0.282 mmol) similar to Example 3 and isolated as a yellow solid (6 mg, 4%). ¹H NMR (CDCl₃): 9.95 (s, 1H), 8.65 (s, 1H), 7.98 (s, 1H), 7.95 (s, 1H), 7.92 (s, 1H), 7.77-7.75 (m, 1H), 7.60-7.57 (m, 1H), 7.52-7.39 (m, 4H), 3.78 (s, 3H), 3.60 (s, 6H), 6.64 (s, 2H).

### EXAMPLE 51

### N,N′-Bis-(3-trifluoromethyl-phenyl)-2-(7-methoxy-benzo[1,3]dioxol-5-yl-methylene)-malonamide

The title compound was prepared from a mixture of N,N′-bis-(3-trifluoromethyl-phenyl)-malonamide (100 mg, 0.256 mmol) and 3-methoxypiperonal (51 mg, 0.282 mmol) similar to Example 3 and isolated as a yellow solid (21 mg, 15%). ¹H NMR (CDCl₃): 9.95 (s, 1H), 8.73 (s, 1H), 7.97 (s, 1H), 7.94 (s, 1H), 7.86 (s, 1H), 7.76-7.73 (m, 1H), 7.69-7.66 (m, 1H), 7.54-7.39 (m, 4H), 6.62-6.59 (m, 2H), 5.90 (s, 2H), 3.64 (s, 3H).

### EXAMPLE 52

### (E) and (Z)-N-(1H-Indol-5-yl)-2-(4-isopropyl-benzylidene)-N′-(3-trifluoromethyl-phenyl)-malonamide

a) N-(1H-Indol-5-yl)-N′-(3-trifluoromethyl-phenyl)-malonamide: To a stirring mixture of N-(3-trifluoromethyl-phenyl)-malonamic acid (200 mg, 0.809 mmol), 1-(3-dimethylaminopropyl)-3-ethylcarbodiimide hydrochloride (171 mg, 0.890 mmol) and 1-hydroxybenzotriazole hydrate (120 mg, 0.890 mmol) in anhydrous tetrahydrofuran (10 mL) was added 5-aminoindole (107 mg, 0.809 mmol). The mixture was stirred at room temperature for 24 h. The solvent was evaporated and the resulting residue was column purified to obtain a tan solid (20 mg, 7). ¹H NMR (CDCl₃): 10.06 (s, 1H), 9.21 (s, 1H), 8.31 (s, 1H), 7.98 (s, 1H), 7.84 (s, 1H), 7.69-7.65 (m, 1H), 7.40-7.17 (m, 4H), 6.47-6.45 (m, 1H), 4.80 (bs, 1H), 3.63 (s, 2H).

b) (E) and (Z)-N-(1H-Indol-5-yl)-2-(4-isopropyl-benzylidene)-N′-(3-trifluoromethyl-phenyl)-malonamide: The title compound was prepared from a mixture of N-(1H-Indol-5-yl)-N′-(3-trifluoromethyl-phenyl)-malonamide (20 mg, 0.055 mmol) and 4-isopropylbenzaldeyhde (10 μL, 0.065 mmol) similar to Example 3 and isolated as yellow solids. Compound A (1 mg, 4%). ¹H NMR (CDCl₃): 9.72 (s, 1H), 8.16-8.12 (m, 2H), 8.08 (s, 1H), 8.03 (s, 1H), 7.65 (s, 1H), 7.59-7.56 (m, 1H), 7.50-7.33 (m, 3H), 7.41 (d, J=7.2 Hz, 2H), 7.24-7.20 (m, 2H), 7.17 (d, J=8.1 Hz, 2H), 6.56-6.54 (m, 1H), 2.89-2.80 (m, 1H), 1.19 (s, 3H), 1.16 (s, 3H). Compound B (1 mg, 4%). ¹H NMR (CDCl₃): 10.22 (s, 1H), 8.20 (s, 1H), 8.11 (s, 1H), 8.00 (s, 1H), 7.85-7.82 (m, 1H), 7.70-7.66 (m, 2H), 7.48 (d, J=8.1 Hz, 2H), 7.44-7.33 (m, 4H), 7.24 (d, J=8.1 Hz, 2H), 7.03 (dd, J=1.8, 8.4 Hz, 1H), 6.56-6.54 (m, 1H), 2.94-2.85 (m, 1H), 1.24 (s, 3H), 1.22 (s, 3H).

### EXAMPLE 53

### N,N′-Bis-(2-chloro-pyridin-4-yl)-2-(4-isopropyl-benzylidene)-malonamide

The title compound was prepared from a mixture of N,N′-bis-(2-chloro-pyridin-4-yl)-malonamide (60 mg, 0.185 mmol) and 4-isopropylbenzaldeyhde (31 μL, 0.204 mmol) similar to Example 3 and isolated as a white solid (4 mg, 5%). ¹H NMR (CDCl₃): 10.05 (s, 1H), 8.31 (d, J=4.2 Hz, 1H), 8.29 (d, J=4.2 Hz, 1H), 8.19 (s, 1H), 8.12 (s, 1H), 7.72 (d, J=1.8 Hz, 1H), 7.49 (d, J=1.5 Hz, 1H), 7.41 (dd, J=1.8, 6.0 Hz, 1H), 7.33 (d, J=8.1 Hz, 2H), 7.22 (d, J=8.1 Hz, 2H), 7.15 (dd, J=2.1, 5.4 Hz, 1H), 2.95-2.85 (m, 1H), 1.23 (s, 3H), 1.20 (s, 3H).

### EXAMPLE 54

### 3-(4-Isopropyl-phenyl)-N-(3-trifluoromethyl-phenyl)-2-(3-trifluoromethyl-phenylsulfamoyl)-acrylamide

a) N-(3-Trifluoromethyl-phenyl)-2-(3-trifluoromethyl-phenylsulfamoyl)-acetamide: To a stirring solution of 3-aminobenzotrifluoride (116 μL, 0.931 mmol) in anhydrous pyridine (5 mL) was slowly added chlorosulfonylacetyl chloride (99 μL, 0.931 mmol). The mixture was stirred at room temperature for 20 h. The solvent was evaporated to dryness to obtain a tan solid (254 mg, 64%). The intermediate was used in the next step without purification.

b) 3-(4-Isopropyl-phenyl)-N-(3-trifluoromethyl-phenyl)-2-(3-trifluoromethyl-phenylsulfamoyl)-acrylamide: The title compound was prepared from a mixture of N-(3-trifluoromethyl-phenyl)-2-(3-trifluoromethyl-phenylsulfamoyl)-acetamide (50 mg, 0.117 mmol) and 4-isopropylbenzaldehyde (20 μL, 0.129 mmol) similar to Example 3 and isolated as an orange solid (4 mg, 6%). ¹H NMR (CDCl₃): 7.73 (s, 1H), 7.70-7.67 (m, 2H), 7.59-7.49 (m, 2H), 7.47 (s, 1H), 7.44-7.41 (m, 4H), 7.30 (d, J=8.1 Hz, 2H), 7.22 (d, J=8.1 Hz, 2H), 7.14 (s, 1H), 2.93-2.84 (m, 1H), 1.22 (s, 3H), 1.19 (s, 3H).

### EXAMPLE 55

### N,N′-Bis-(3-trifluoromethyl-phenyl)-2-(6-trifluoromethyl-pyridin-3-yl-methylene)-malonamide

The title compound was prepared from a mixture of N,N′-bis-(3-trifluoromethyl-phenyl)-malonamide (100 mg, 0.256 mmol) and 6-(trifluoromethyl)pyridine-3-carboxaldehyde (45 mg, 0.256 mmol) similar to Example 3 and isolated as a white solid (14 mg, 10%). ¹H NMR (CDCl₃): 9.94 (s, 1H), 9.39 (s, 1H), 8.67 (s, 1H), 7.98 (s, 1H), 7.93 (s, 1H), 7.86-7.83 (m, 1H), 7.76 (s, 1H), 7.74-7.71 (m, 1H), 7.66-7.62 (m, 1H), 7.54-7.45 (m, 5H).

### EXAMPLE 56

### (E) and (Z)-N-(2-Chloro-pyridin-4-yl)-2-(4-isopropyl-benzylidene)-N′-(3-trifluoromethyl-phenyl)-malonamide

a) N-(2-Chloro-pyridin-4-yl)-N′-(3-trifluoromethyl-phenyl)-malonamide: To a stirring solution of N-(3-trifluoromethyl-phenyl)-malonamic acid (1 g, 4.05 mmol) in anhydrous dichloromethane (100 mL) was added phosphorus pentachloride (842 mg, 4.05 mmol). The mixture was stirred at room temperature for 30 minutes. Then 4-amino-2-chloropyridine (521 mg, 4.05 mmol) and triethylamine (563 μL, 4.05 mmol) was added and the mixture was stirred at room temperature for 20 h. The solvent was evaporated and isolated as a tan solid (790 mg, 55%). ¹H NMR (CDCl₃): 9.69 (s, 1H), 8.69 (s, 1H), 8.31 (d, J=5.7 Hz, 1H), 7.92 (s, 1H), 7.73 (d, J=1.8 Hz, 1H), 7.71-7.67 (m, 1H), 7.53-7.44 (m, 2H), 7.39 (dd, J=2.1, 5.4 Hz, 1H), 3.61 (s, 2H).

b) E) and (Z)-N-(2-Chloro-pyridin-4-yl)-2-(4-isopropyl-benzylidene)-N′-(3-trifluoromethyl-phenyl)-malonamide: The title compound was prepared from a mixture of N′-(3-trifluoromethyl-phenyl)-N-(2-chloro-pyridin-4-yl)-malonamide (790 mg, 2.21 mmol) and 4-isopropylbenzaldeyhde (369 μL, 2.43 mmol) similar to Example 3 and isolated as a white solid (164 mg, 15%).

### EXAMPLE 57

### (E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide

a) 4-[2-(4-Nitro-2-trifluoromethyl-phenoxy)-ethyl]-morpholine: A mixture of N-(2-hydroxyethyl)morpholine (6.97 mL, 57.4 mmol) and potassium-tert-butoxide (6.44 g, 57.4 mmol) in anhydrous THE (40 mL) was stirred at room temperature for 30 minutes. Then 2-fluoro-5-nitrobenzotrifluoride (6.57 mL, 47.8 mmol) was added and the mixture was stirred at room temperature for 4 h. The solvent was evaporated and the resulting residue was diluted with ethylacetate (350 mL), wash with water (2×50 mL), brine (1×50 mL) and dried over anhydrous sodium sulfate. The solvent was evaporated to obtain a yellow solid (14.6 g, 96%). ¹H NMR (CDCl₃): 8.52 (d, J=2.7 Hz, 1H), 8.43 (dd, J=2.7, 9.0 Hz, 1H), 7.11 (d, J=9.3 Hz, 1H), 4.32 (t, J=5.4 Hz, 2H), 3.72 (t, J=4.8 Hz, 4H), 2.90 (t, J=5.4 Hz, 2H), 2.61 (t, J=4.5 Hz, 4H).

b) 4-(2-Morpholin-4-yl-ethoxy)-3-trifluoromethyl-phenylamine: To a solution of 4-[2-(4-nitro-2-trifluoromethyl-phenoxy)-ethyl]-morpholine in methanol (60 mL) was added palladium (5 wt % on activated carbon). The mixture was hydrogenated at 50 psi for 4 h. The mixture was filtered over celite and wash with methanol. The solvent was evaporated to dryness to obtain a brown oil (6.3 g, 99%). ¹H NMR (CDCl₃): 6.90 (d, J=2.4 Hz, 1H), 6.84 (d, J=8.7 Hz, 1H), 6.78 (dd, J=2.7, 8.7 Hz, 1H), 4.10 (t, J=5.7 Hz, 2H), 3.72 (t, J=4.2 Hz, 4H), 3.56 (s, 2H), 2.80 (t, J=5.7 Hz, 2H), 2.59 (t, J=4.5 Hz, 4H).

c) N-[4-(2-Morpholin-4-yl-ethoxy)-3-trifluoromethyl-phenyl]-N′-(3-trifluoromethyl-phenyl)-malonamide: The title compound was prepared from a mixture of N-(3-trifluoromethyl-phenyl)-malonamic acid (4 g, 16.2 mmol) and 4-(2-morpholin-4-yl-ethoxy)-3-trifluoromethyl-phenylamine (4.7 g, 16.2 mmol) similar to Example 58a and isolated as an orange solid (7.1 g, 85%). %). ¹H NMR (CDCl₃): 9.67 (s, 1H), 9.37 (s, 1H), 7.97 (s, 1H), 7.81 (d, J=2.7 Hz, 1H), 7.72-7.66 (m, 2H), 7-47 (d, J=7.5 Hz, 1H), 7.41 (dd, J=2.4, 8.1, 1H), 6.96 (d, J=9.3 Hz, 1H), 4.18 (t, J=5.7, 2H), 3.72 (t, J=4.8 Hz, 4H), 3.64 (s, 2H), 2.84 (t, J=5.7 Hz, 2H), 2.60 (t, J=4.8 Hz, 4H).

d) (E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide: The title compound was prepared from a mixture of N′-(3-trifluoromethyl-phenyl)-N-[4-(2-morpholin-4-yl-ethoxy)-3-trifluoromethyl-phenyl]-malonamide (3 g, 5.78 mmol) and 4-isopropylbenzaldehyde (964 μL, 6.35 mmol) similar to Example 3 and isolated as a yellow solid (1.1 g, 29%).

### EXAMPLE 58

### (E) and (z)-N-(2-Chloro-pyridin-4-yl)-2-(2,3-dihydro-benzo[1,4]dioxin-6-yl-methylene)-N′-(3-trifluoromethyl-phenyl)-malonamide

The title compound was prepared from a mixture of N′-(3-trifluoromethyl-phenyl)-N-(2-chloro-pyridin-4-yl)-malonamide (100 mg, 0.280 mmol) and 1,4-benzodioxan-6-carboxaldehyde (46 mg, 0.280 mmol) similar to Example 3 and isolated as a yellow solid (32 mg, 23%).

### EXAMPLE 59

### (E) and (Z)-2-Benzylidene-N-(2-chloro-pyridin-4-yl)-N′-(3-trifluoromethyl-phenyl)-malonamide

The title compound was prepared from a mixture of N′-(3-trifluoromethyl-phenyl)-N-(2-chloro-pyridin-4-yl)-malonamide (100 mg, 0.280 mmol) and benzaldehyde (29 μL, 0.280 mmol) similar to Example 3 and isolated as a yellow solid (21 mg, 17%).

### EXAMPLE 60

### (E) and (Z)-2-(4-Chloro-benzylidene)-N-(2-chloro-pyridin-4-yl)-N′-(3-trifluoromethyl-phenyl)-malonamide

The title compound was prepared from a mixture of N′-(3-trifluoromethyl-phenyl)-N-(2-chloro-pyridin-4-yl)-malonamide (100 mg, 0.280 mmol) and 4-chlorobenzaldehyde (39 mg, 0.280 mmol) similar to Example 3 and isolated as a tan solid (20 mg, 15%).

### EXAMPLE 61

### (E) and (Z)-N-(2-Dimethylamino-ethyl)-2-(4-isopropyl-benzylidene)-N′-(3-trifluoromethyl-phenyl)-malonamide

a) N-(2-Dimethylamino-ethyl)-N′-(3-trifluoromethyl-phenyl)-malonamide: The title compound was prepared from a mixture of N-(3-trifluoromethyl-phenyl)-malonamic acid (500 mg, 2.02 mmol) and N,N-dimethylethylenediamine (222 μL, 2.02 mmol), similar to Example 58a and isolated as an orange solid (429 mg, 67%). ¹H NMR (CDCl₃): 10.08 (s, 1H), 7.88 (s, 1H), 7.77-7.74 (m, 1H), 7.45-7.40 (m, 1H), 7.36-7.33 (m, 1H), 6.75 (s, 1H) 3.39 (t, J=6.0 Hz, 2H), 3.35 (s, 2H), 2.46 (t, J=6.0 Hz, 2H), 2.25 (s, 6H).

b) (E) and (Z)-N-(2-Dimethylamino-ethyl)-2-(4-isopropyl-benzylidene)-N′-(3-trifluoromethyl-phenyl)-malonamide: The title compound was prepared from a mixture of N-(2-dimethylamino-ethyl)-N′-(3-trifluoromethyl-phenyl)-malonamide (100 mg, 0.315 mmol) and 4-isopropylbenzaldehyde (53 μL, 0.347 mmol) similar to Example 3 and isolated as a yellow solid (5 mg, 4%). ¹H NMR (CDCl₃): 10.05 (s, 1H), 8.00 (s, 1H), 7.96 (s, 1H), 7.84 (d, J=8.1 Hz, 1H), 7.45 (t, J=7.8 Hz, 1H), 7.38 (d, J=8.4 Hz, 2H), 7.37-7.35 (m, 1H), 7.26 (d, J=8.4 Hz, 2H), 6.47 (s, 1H), 3.31 (q, J=5.4 Hz, 2H), 2.97-2.88 (m, 1H), 2.21 (t, J=5.7 Hz, 2H), 1.92 (s, 6H), 1.26 (s, 3H), 1.23 (s, 3H).

### EXAMPLE 62

### (E) and (Z)-3-[3-(4-Isopropyl-phenyl)-2-(3-trifluoromethyl-phenylcarbamoyl)-acryloylamino]-benzoic acid

a) 3-[2-(3-Trifluoromethyl-phenylcarbamoyl)-acetylamino]-benzoic acid ethyl ester: The title compound was prepared from a mixture of N-(3-trifluoromethyl-phenyl)-malonamic acid (400 mg, 1.62 mmol) and ethyl 3-aminobenzoate (241 μL, 1.62 mmol) similar to Example 56a and isolated as a yellow solid (580 mg, 91%).

b) 3-[2-(3-Trifluoromethyl-phenylcarbamoyl)-acetylamino]-benzoic acid: The title compound was prepared from 3-[2-(3-trifluoromethyl-phenylcarbamoyl)-acetylamino]-benzoic acid ethyl ester similar to Example 33b and isolated as a white solid (490 mg, 92%).

c) (E) and (Z)-3-[3-(4-Isopropyl-phenyl)-2-(3-trifluoromethyl-phenylcarbamoyl)-acryloylamino]-benzoic acid: The title compound was prepared from a mixture of 3-[2-(3-trifluoromethyl-phenylcarbamoyl)-acetylamino]-benzoic acid (200 mg, 0.546 mmol) and 4-isopropylbenzaldehyde (92 μL, 0.601 mmol) similar to Example 3 and isolated as a white solid 46 mg, 17%).

### EXAMPLE 63

### (E) and (Z)-N-(2-Chloro-1-oxy-pyridin-4-yl)-2-(4-isopropyl-benzylidene)-N′-(3-trifluoromethyl-phenyl)-malonamide

a) N-(2-Chloro-1-oxy-pyridin-4-yl)-N′-(3-trifluoromethyl-phenyl)-malonamide: To a stirring solution of N-(2-chloro-pyridin-4-yl)-N′-(3-trifluoromethyl-phenyl)-malonamide (100 mg, 0.280 mmol) in anhydrous dichloromethane (5 mL) was added m-chloroperbenzoic acid (73 mg, 0.420 mmol). The mixture was stirred at room temperature for 24 h. The solid was collected by filtration and washed with hexanes to obtain a white solid (80 mg, 77%). ¹H NMR (DMSO-d₆): 10.83 (s, 1H), 10.57 (s, 1H), 8.36 (dd, J=1.5, 7.5 Hz, 1H), 8.10 (s, 1H), 8.04 (dd, J=1.2, 3.3 Hz, 1H), 7.77 (d, J=6.9 Hz, 1H), 7.58 (t, J=7.5 Hz, 1H), 7.52-7.49 (m, 1H), 7.44 (d, J=7.8 Hz, 1H), 3.56 (s, 2H).

b) (E) and (Z)-N-(2-Chloro-1-oxy-pyridin-4-yl)-2-(4-isopropyl-benzylidene)-N′-(3-trifluoromethyl-phenyl)-malonamide: The title compound was prepared from a mixture of N-(2-chloro-1-oxy-pyridin-4-yl)-N′-(3-trifluoromethyl-phenyl)-malonamide (80 mg, 0.214 mmol) and 4-isopropylbenzaldehyde (36 μL, 0.235 mmol) similar to Example 3 and isolated as a yellow solid (7 mg, 7%).

### EXAMPLE 64

### N,N′-Bis-(3-trifluoromethyl-phenyl)-2-(4-dimethyamino-benzylidene)-malonamide

The title compound was prepared from a mixture of N,N′-bis-(3-trifluoromethyl-phenyl)-malonamide (50 mg, 0.128 mmol) and 4-dimethylaminobenzaldehyde (30 mg, 0.166 mmol) similar to Example 3 and isolated as a yellow solid (10 mg, 20%). ¹H NMR (CDCl₃): 10.17 (s, 1H), 9.53 (s, 1H), 8.08 (s, 1H), 7.94 (brs, 1H), 7.88 (s, 1H), 7.81(d, J=7.5 Hz, 1H), 7.62 (d, J=7.5 Hz, 1H), 7.51-7.32 (m, 5H), 7.28-7.25(m, 3H), 3.92 (s, 6H).

### EXAMPLE 65

### N,N′-Bis-(3-trifluoromethyl-phenyl)-2-(3-nitro-5-chloro-benzylidene)-malonamide

The title compound was prepared from a mixture of N,N′-bis-(3-trifluoromethyl-phenyl)-malonamide (50 mg, 0.128 mmol) and 3-nitro-5-chlorobenzaldehyde (30 mg, 0.172 mmol) similar to Example 3 and isolated as a yellow solid (12 mg, 24%). ¹H NMR (CDCl₃): 9.49 (s, 1H), 9.31 (s, 1H), 8.08-8.04 (brd, 3H), 7.84-7.79 (m, 2H), 7.64-7.40 (m, 7H).

### EXAMPLE 66

### N,N′-Bis-(3-trifluoromethyl-phenyl)-2-(4-methoxy-benzylidene)-malonamide

The title compound was prepared from a mixture of N,N′-bis-(3-trifluoromethyl-phenyl)-malonamide (50 mg, 0.128 mmol) and 4-methoxybenzaldehyde (20 μl, 147 mmol) similar to Example 3 and isolated as a white solid (15 mg, 30%). ¹H NMR (CDCl₃): 10.08 (s, 1H), 9.16 (s, 1H), 8.08 (s, 1H), 7.99 (brs, 1H), 7.94 (brs, 1H), 7.89 (s, 1H), 7.69-7.66 (m, 2H), 7.50-7.32(m, 6H), 6.69 (d, J=8.7 Hz, 2H), 3.68 (s, 3H).

### EXAMPLE 67

### N,N′-Bis-(3-trifluoromethyl-phenyl)-2-(4-chloro-benzylidene)-malonamide

The title compound was prepared from a mixture of N,N′-bis-(3-trifluoromethyl-phenyl)-malonamide (50 mg, 0.128 mmol) and 4-chlorobenzaldehyde (20 mg, 0.143 mmol) similar to Example 3 and isolated as a white solid (11 mg, 22%). ¹H NMR (CDCl₃): 10.04 (s, 1H), 9.23 (s, 1H), 8.00 (s, 1H), 7.94 (brs, 1H), 7.82 (s, 1H), 7.69-7.61 (m, 2H), 7.54-7.42(m, 4H), 7.29(d, J=8.1 Hz, 2H), 7.11(d, J=8.1 Hz, 2H).

### EXAMPLE 68

### N,N′-Bis-(3-trifluoromethyl-phenyl)-2-(3,5-dichloro-benzylidene)-malonamide

The title compound was prepared from a mixture of N,N′-bis-(3-trifluoromethyl-phenyl)-malonamide (50 mg, 0.128 mmol) and 3,5-dichlorobenzaldehyde (30 mg, 0.171 mmol) similar to Example 3 and isolated as an off white solid (9 mg, 18%). ¹H NMR (CDCl₃): 10.16 (s, 1H), 9.27 (s, 1H), 8.03 (s, 1H), 7.92 (s, 1H), 7.75-7.65 (m, 4H), 7.54-7.46 (m, 4H), 7.23-7.21 (brd, J=7.5 Hz, 2H).

### EXAMPLE 69

### N,N′-Bis-(3-trifluoromethyl-phenyl)-2-(2,6-dichloro-benzylidene)-malonamide

The title compound was prepared from a mixture of N,N′-bis-(3-trifluoromethyl-phenyl)-malonamide (5O mg, 0.128 mmol) and 2,6-dichlorobenzaldehyde (26 mg, 0.15 mmol) similar to Example 3 and isolated as an off white solid (48 mg, 96%). ¹H NMR (CDCl₃): 10.34 (s, 1H), 8.55 (s, 1H), 8.07 (s, 1H), 7.89 (s, 1H), 7.86(s, 1H), 7.62 (brs, 1H), 7.52-7.39 (m, 5H), 7.25 (brd, J=8.4 Hz, 2H), 7.09 (dd, J=8.7, and 8.4 Hz, 1H).

### EXAMPLE 70

### N,N′-Bis-(3-trifluoromethyl-phenyl)-2-(5-bromo-2-furyl-methylene)-malonamide

The title compound was prepared from a mixture of N,N′-bis-(3-trifluoromethyl-phenyl)-malonamide (50 mg, 0.128 mmol) and 5-bromofuraladehyde (26 mg, 0.15 mmol) similar to Example 3 and isolated as an off white solid (19 mg, 38%). ¹H NMR (CDCl₃): 9.96 (s, 1H), 9.93 (s, 1H), 8.16 (s, 1H), 7.98(d, J=8.4 Hz, 1H), 7.90 (s, 1H), 7.67 (d, J=8.4 Hz, 1H), 7.61-7.335 (m, 7H).

### EXAMPLE 71

### N,N′-Bis-(3-trifluoromethyl-phenyl)-2-[(5-bromo-2-furyl)-hydroxy-methyl]-malonamide

The title compound is was isolated as a side product from the preparation of compound of Example 70 as white solid (5 mg, 10%). ¹H NMR (CDCl₃): ¹H NMR (CDCl₃): 9.96 (s, 1H), 9.93 (s, 1H), 7.85 (s, 1H), 7.73 (d, J=8.1 Hz, 1H), 7.59 (d, J=8.1 Hz, 1H), 7.48-7.35 (m, 5H), 6.26 (m, 1H), 4.53(d, J=8.1 Hz, 1H), 4.04 (d, J=8.1 Hz, 1H).

### EXAMPLE 72

### N,N′-Bis-(3-trifluoromethyl-phenyl)-2-[(5-bromo-2-furyl)-1-piperidinyl-methyl]-malonamide

The title compound was isolated as a white solid (10 mg, 20%) during the preparation of compound of example 70. ¹H NMR (CDCl₃): 10.27 (s, 1H), 9.23 (s, 1H), 8.35 (s, 1H), 8.06 (d, J=8.4 Hz, 1H), 7.78 (s, 1H), 7.59 (d, J=8.4 Hz, 1H), 7.52 (t, J=8.4 Hz, 1H), 7.42-7.25 (m, 4H), 6.79 (J=4.4 Hz, 1H), 4.94(d, J=4.3 Hz, 1H), 2.88 (m, 4H), 1.33 (m, 2H), 1.26 (m, 4H).

### EXAMPLE 73

### N,N′-Bis-(3-trifluoromethyl-phenyl)-2-(2,3-dichloro-benzylidene)-malonamide

The title compound was prepared from a mixture of N,N′-bis-(3-trifluoromethyl-phenyl)-malonamide (50 mg, 0.128 mmol) and 2,3-dichlorobenzaldehyde (26 mg, 0.15 mmol) similar to Example 3 and isolated as a white solid (34 mg, 68%). ¹H NMR (DMSO-4): 10.84 (s, 1H), 10.56 (s, 1H), 8.18 (s, 1H), 8.02 (brs, 1H), 7.98 (s, 1H), 7.80 (s, 1H), 7.73 (d, J=8.4 Hz, 1H), 6.66 (dd, J=1.2, 7.8 Hz, 1H), 7.65-7.44 (m, 6H), 7.36 (dd, J=7.6, 8.1 Hz, 1H).

### EXAMPLE 74

### N,N′-Bis-(3-trifluoromethyl-phenyl)-2-(4-N-methylpiperidinyl-benzylidene)-malonamide

The title compound was prepared from a mixture of N,N′-bis-(3-trifluoromethyl-phenyl)-malonamide (50 mg, 0.128 mmol) and 4-N-methylpiperidinebenzaldehyde (56 mg, 0.273 mmol) similar to Example 3 and isolated as a light yellow solid (14 mg, 28%). ¹H NMR (CDCl₃): 10.01 (s, 1H), 8.67 (s, 1H), 8.04 (s, 1H), 7.88 (m, 2H), 7.77 (s, 1H), 7.51-7.31 (m, 7H), 6.89 (d, J=8.7 Hz, 1H), 6.71 (d, J=8.7 Hz, 1H), 3.30-3.21(m, 4H), 2.52-2.49 (m, 4H), 2.34 (s, 3H).

### EXAMPLE 75

### N,N′-Bis-(3-trifluoromethyl-phenyl)-2-(6-bromo-pyridin-2-yl-methylene)malonamide

The title compound was prepared from a mixture of N,N′-bis-(3-trifluoromethyl-phenyl)-malonamide (50 mg, 0.128 mmol) and 6-bromo-2-pyridinecarboxyaldehyde (30 mg, 0.16 mmol) similar to Example 3 and isolated as an off white solid (14 mg, 28%). ¹H NMR (CDCl₃): 10.77 (s, 1H), 10.67 (s, 1H), 8.12(s, 1H), 7.99 (brd, 2H), 7.94 (s, 1H), 7.74 (d, J=7.8 Hz, 1H), 7.56-7.38 (m, 7H).

### EXAMPLE 76

### N,N′-Bis-(3-trifluoromethyl-phenyl)-2[(6-bromo-pyridin-2-yl)-hydroxy-methyl]malonamide

The title compound was prepared from a mixture of N,N′-bis-(3-trifluoromethyl-phenyl)-malonamide (50 mg, 0.128 mmol) and 6-bromo-2-pyridinecarboxyaldehyde (30 mg, 0.16 mmol) similar to Example 3 and isolated using a small silica column as an off white solid (14 mg, 28%). ¹H NMR (CDCl₃): 7.96 (s, 1H), 7.82 (s, 1H), 7.45 (d, J=7.8 Hz, 1H), 7.55 (d, J=7.8 Hz, 1H), 7.48-7.30 (m, 7H), 7.20 (d, J=6.2 Hz, 1H), 4.48 (m, 1H), 4.21 (d, J=6.2 Hz, 1H).

### EXAMPLE 77

### N,N′-Bis-(3-trifluoromethyl-phenyl)-2-(3-bromo-4,5-dimethoxy-benzylidene)-malonamide

The title compound was prepared from a mixture of N,N′-bis-(3-trifluoromethyl-phenyl)-malonamide (50 mg, 0.128 mmol) and 4-bromo-4,5-dimethoxybenzaldehyde (38 mg, 0.154 mmol) similar to Example 3 and isolated as an off white solid (8 mg, 16%). ¹H NMR (CDCl₃): 9.97 (s, 1H), 8.76 (s, 1H), 8.00 (s, 1H), 7.85 (s, 1H), 7-83 (d, J=8.1 Hz, 1H), 7.65 (ddd, J=2.1, 4.2 and 8.8 Hz, 1H), 7.54-7.43 (m, 4H), 7.29 (s, 1H), 7.21 (d, J=2.1 Hz, 1H), 6.91 (d, J=2.1 Hz, 1H), 3.79 (s, 3H), 3.60 (s, 3H).

### EXAMPLE 78

### N,N′-Bis-(3-trifluoromethyl-phenyl)-2-[(3-bromo-4,5-dimethoxy-phenyl)-hydroxy-methyl]-malonamide

The title compound was isolated as a side product from the preparation of compound of Example 77 as an off white solid (9 mg, 18%). ¹H NMR (CDCl₃): 9.89 (s, 1H), 9.38 (s, 1H), 9.33 (s, 1H), 7.93 (s, 1H), 7.91 (brd, 1H), 7.70-7.63 (m, 3H), 7.50-7.38 (m, 5H), 4.25 (brt, 1H), 4.40 (brd, 1H), 3.71 (s, 3H), 3.68 (s, 3H).

### EXAMPLE 79

### N,N′-Bis-(3-trifluoromethyl-phenyl)-2-(3,5-dimethoxy-4-hydroxy-benzylidene)-malonamide

The title compound was prepared from a mixture of N,N′-bis-(3-trifluoromethyl-phenyl)-malonamide (50 mg, 0.128 mmol) and 3,5-dimethoxy-4-hydroxybenzaldehyde (35 mg, 0.154 mmol) similar to Example 3 and isolated as an white solid mass (16 mg, 32%). ¹H NMR (CDCl₃+CD₃OD): 8.01 (s, 1H), 7.83 (s, 2H), 7.74 (d, J=8.1 Hz, 1H), 7.68 (d, J=7.8 Hz, 1H), 7.51-7.36 (m, 5H), 7.12 (bs, 2H), 3.85 (s, 6H).

### EXAMPLE 80

### N,N′-Bis-(3-trifluoromethyl-phenyl)-2-(3-bromo-4-methoxy-benzylidene)malonamide

The title compound was prepared from a mixture of N,N′-bis-(3-trifluoromethyl-phenyl)-malonamide (50 mg, 0.128 mmol) and 3-bromo-4-methoxybenzaldehyde (33 mg, 0.154 mmol) similar to Example 3 as a solid (3 mg, 6%). ¹H NMR (CDCl₃): 10.05 (s, 1H), 9.16 (s, 1H), 7.98 (s, 1H), 7.95 (m, 2H), 7.78 (s, 1H), 7.73-7.67 (m, 2H), 7.55-7.35 (m, 5H), 6.66 (d, J=8.7 Hz, 1H), 3.77 (s, 3).

### EXAMPLE 81

### N,N′-Bis-(3-trifluoromethyl-phenyl)-2-[(3-bromo-4-methoxy-phenyl)-hydroxy-methyl]-malonamide

The title compound was isolated as a side product from the preparation of compound of Example 80 as a white solid (8 mg, 16%). ¹H NMR (CDCl₃+CD₃OD): 7.88 (s, 1H), 7.74-7.67 (m, 2H), 7.54 (s, 1H), 7.59-7.33 (m, 7H), 6.15 (d, J=7.7 Hz, 1H), 6.75 (d, J=7.7 Hz, 1H), 4.36 (d, J=7.8 Hz, 1H), 3.99 (d, J=7.8 Hz, 1H), 3.65(s, 3H).

### EXAMPLE 82

### N,N′-Bis-(6-bromo-pyridin-2-yl)-2-(4-isopropyl-benzylidene)-malonamide

a) N,N′-Bis-(6-bromo-2-pyridyl)-malonamide: To a stirring solution of 2-amino-6-bromo-pyridine (300 mg, 1.73 mmol) in 1,4-dioxane (5 ml) was slowly added malonyl dichloride (140 μl, 0.86 mmol). The mixture was stirred at room temperature for 15 min. The mixture was diluted with water (50 ml) and the resulting solid was collected by filtration. The solid was washed with excess water and then dried under vacuum at 45° C. to give a light brown solid (250 mg, 83%). ¹H NMR (CDCl₃): 9.24 (s, 2H), 8.22 (d, J=7.1 Hz, 2H), 7.59 (t, J=7.1 Hz, 2H), 7.29 (m, 4H), 3.61 (s, 2H)

b) N,N′-Bis-(6-bromo-2-pyridyl)-2-(4-isopropyl-benzylidene)-malonamide: The title compound was prepared from a mixture of N,N′-bis-(6-bromo-pyridiny)-malonamide (30 mg, 0.07 mmol) and 4-isopropylbenzaldehyde (20 ul, 0.132 mmol) similar to Example 3 as a solid (10 mg, 33%). ¹H NMR (CDCl₃): 9.74 (s, 1H), 8.35 (d, J=7.8 Hz, 1H), 8.25 (d, J=7.8 Hz, 1H), 8.08 (s, 1H), 8.07 (s, 1H), 7.66-7.552H), 7.41 (d, J=7.8 Hz, 2H), 7.29-7.21 (m, 4H), 2.90 (sep, J=6.9 Hz, 1H), 1.22 (d, J=6.9 Hz, 6H).

### EXAMPLE 83

### N,N′-Bis-(3-carboxy-phenyl)-2-benzylidene-malonamide

a) N,N′-Bis-(3-carboxy-phenyl)-malonamide: To a stirring solution of 3-amino-benzoic acid (500 mg, 3.65 mmol) in 1,4-dioxane (5 ml) was slowly added malonyl dichloride (140 μl, 1.83 mmol). The mixture was stirred at room temperature for 15 min. The mixture was diluted with water (50 ml) and the resulting solid was collected by filtration. The solid was washed with excess water and then dried under vacuum at 45° C. to give off white solid (500 mg, 100%). ¹H NMR (DMSO-d₆): 10.40 (s, 1H), 8.26 (s, 2H), 7.85(d, J=7.5 Hz, 2H), 7.64 (d, J=6.9 Hz, 2H), 7.45 (m, 2H), 3.52 (s, 2H).

b) N,N′-Bis-(3-carboxypheny)-2-benzylidene-malonamide: The title compound was prepared from a mixture of N,N′-bis-(3-carboxypheny)malonamide (30 mg, 0.08 mmol) and benzaldehyde (20 ul, 0.17 mmol) in pyridine. The reaction mixture was refluxed at 85-90° C. for 12 hours. After completion, pyridine was removed under reduced pressure. The dark brown residue was redissolved in 3-ml water and basified with 1N NH₄OH. The aqueous solution was extracted with ethylacetate (10 ml×3). It was then acidified with 1N HCl to pH 3. Precipitate was observed which was filtered, washed with water and dried under vacuum to give a tan solid (5 mg, 16%). ¹H NMR (DMSO-D₆): 10.16 (s, 1H), 10.69 (s, 1H), 10.39 (s, 1H), 10.32 (s, 1H), 8.33 (brd, 1H), 8.26 (t, J=2.1 Hz, 1H), 7.86-7.82 (m, 2H), 7.68-7.39 (m, 8H).

### EXAMPLE 84

### N,N′-Bis-(3-chloro-phenyl)-2-(3-chloro-5-nitro-benzylidene)-malonamide

a) N,N′-Bis-(3-chloro-phenyl)-malonamide: The title compound was prepared from a mixture of 3-chloroaniline (323 μl, 3.55 mmol) and malonyl dichloride (138 μL, 1.42 mmol) similar to Example 1a and isolated as a light yellow solid (222 mg, 62%). ¹H NMR (CDCl₃): 8.73 (s, 2H), 7.56 (d, J=7.8 Hz, 4H), 7.36 (t, J=8.0 Hz, 4H), 7.19-7.14 (m, 2H), 3.54 (s, 2H).

b) N,N′-Bis-(3-chloro-phenyl)-2-(3-chloro-5-nitro-benzylidene)-malonamide: The title compound was prepared from a mixture of N,N′-Bis-(3-chloro-phenyl)-malonamide (50 mg, 0.155 mmol) and 3-chloro-5-nitrobenzaldehyde (34 mg, 0.186 mmol) similar to Example 3 and isolated as a yellow solid (26 mg, 52%). ¹H NMR (CDCl₃): 8.67 (s, 1H), 8.65 (s, 1H), 7.75 (brd, 1H), 7.55-7.39 (m, 5H), 7.18-6.98 (m, 6H).

### EXAMPLE 85

### N,N′-Bis-(3-chloro-phenyl)-2-(3-bromo-4,5-dimethoxy-benzylidene)-malonamide

The title compound was prepared from a mixture of N,N′-bis-(3-chloro-phenyl)-malonamide (50 mg, 0.155 mmol) and 3-bromo-4,5-dimethoxybenzaldehyde (45 mg, 0.186 mmol) similar to Example 3 and isolated as a yellow solid (20 mg, 40%). ¹H NMR (CDCl₃): 9.84 (s, 1H), 9.23 (s, 1H), 7.77 (t, J=1.5 Hz, 1H), 7.68 (t, J=1.5 Hz, 1H), 7.66(s, 1H), 7.42-7.11 (m, 7H), 6.85 (d, J=2.1 Hz, 1H), 3.76 (s, 3H), 3.54 (s, 3H).

### EXAMPLE 86

### N,N′-Bis-(3-chloro-phenyl)-2-[(4-trifluoromethyl-phenyl)-1-piperidinyl-methyl]-malonamide

The title compound was prepared from a mixture of N,N′-Bis-(3-chloro-phenyl)-malonamide (50 mg, 0.155 mmol) and 4-trifluoromethyl-benzaldehyde (20 μL, 0.186 mmol) similar to Example 3 and isolated as a yellow solid (24 mg, 48%). ¹H NMR (CDCl₃): 7.75 (brs, 1H), 7.62 (s, 1H), 7.59 (s, 1H), 7.438-7.26 (m, 5H), 7.15-7.11 (m, 3H), 7.02 (m, 1H), 4.53(d, J=8.7 Hz, 1H), 4.17 (d, J=8.7 Hz, 1H), 2.56 (m, 2H), 2.35 (m, 2H), 1.60 (m, 5H), 1.35 (m, 1H).

### EXAMPLE 87

### (E) and (Z)-2-(3-Trifluoromethyl-benzylidene)-N-(2-chloro-4-pyridyl)-N′-(3-trifluoromethyl-phenyl)-malonamide

a) N-(2-Chloro-pyridyl)-N′-(3-trifluoromethyl-phenyl)-malonamide: To a stirring solution of N-(3-trifluoromethyl-phenyl)-malonamic acid as prepared in example 33a and 33b (1 g, 4.05 mmol) in anhydrous dichloromethane (100 mL) was added phosphorus pentachloride (842 mg, 4.05 mmol). The mixture was stirred at room temperature for 30 minutes. Then 4-amino-2-chloropyridine (521 mg, 4.05 mmol) was added and the mixture was stirred at room temperature for 20 h. The reaction mixture was diluted with 50 ml dichloromethane and washed with 5% sodium bicarbonate. The organic solvent was evaporated and product was isolated as off white solid from a small silica column (790 mg, 55%). ¹H NMR (CDCl₃): 9.69 (s, 1H), 8.69 (s, 1H), 8.31 (d, J=5.7 Hz, 1H), 7.92 (s, 1H), 7.73 (d, J=1.8 Hz, 1H), 7.71-7.67 (m, 1H), 7.53-7.44 (m, 2H), 7.39 (dd, J=2.1, 5.4 Hz, 1H), 3.61 (s, 2H).

b) (E) and (Z)-2-(3-trifluoromethyl-benzylidene)-N-(2-chloro-4-pyridyl)-N′-(3-trifluoromethyl-phenyl)-malonamide: The title compound was prepared from a mixture of N-(2-chloro-4-pyridyl)-N′-(3-trifluoromethyl-phenyl)-malonamide (50 mg, 0.14 mmol) and 3-trifluoromethylbenzaldehyde (20 μL, 0.78 mmol) similar to Example 3 and isolated as a solid. ¹H N (CDCl₃): 9.92 (s, 1H), 9.51 (s, 1H), 8.73 (s, 1H), 8.18 (t, J=5.1 Hz, 1H), 7.93 (s, 1H), 7.84(s, 1H), 7.62-7.32 (m, 16H), 7.18-7.09 (m, 4H).

### EXAMPLE 88

### (E) and (Z)-2-(3-Bromo-4,5-dimethoxy-benzylidene)-N-(2-chloro-4-pyridyl)-N′-(3-trifluoromethyl-phenyl)-malonamide

The title compound was prepared from a mixture of N-(2-chloro-4-pyridyl)-N′-(3-trifluoromethyl-phenyl)-malonamide (100 mg, 0.28 mmol) and 3-bromo-4,5-dimethoxybenzaldehyde (100 mg 0.392 mmol) similar to Example 3 and isolated as a solid (8 mg, 8%). ¹H NMR (CDCl₃): 10.12 (s, 1H), 9.82 (s, 1H), 9.50 (s, 1H), 8.77(s, 1H), 8.35 (d, J=5.7 Hz, 1H), 8.29 (d, J=5.4 Hz, 1H), 7.96 (s, 1H), 7.91 (s, 1H), 7.80-7.32 (m, 12H), 7.12 (d, J=2.1 Hz, 1H), 7.06 (d, J=2.1 Hz, 1H), 6.88 (d, J=2.1 Hz, 1H), 6.81 (d, J=2.1 Hz, 1H), 3.94(s, 3H), 3.91(s, 3H), 3.78(s, 3H), 3.74(s, 3H).

### EXAMPLE 89

### (E) and (Z)-2-(3-Pyridyl-methylene)-N-(2-chloro-4-pyridyl)-N′-(3-trifluoromethyl-phenyl)-malonamide

The title compound was prepared from a mixture of N-(2-chloro-4-pyridyl)-N′-(3-trifluoromethyl-phenyl)-malonamide (100 mg, 0.28 mmol) and 3-pyridinecarboxyaldehyde (30 μL, 0.336 mmol) similar to Example 3 and isolated as a solid (12 mg, 12%). ¹H NMR (CDCl₃): 10.11 (s, 1H), 10.08 (s, 1H), 9.72 (s, 1H), 9.34 (s, 1H), 8.86 (s, 1H), 8.72-8.66 (m, 3H), 8.45-8.36 (m, 3H), 8.07-7.86 (m, 6H), 7.70-7.37 (m, 7H).

### EXAMPLE 90

### (E) and (Z)-2-(6-Chloro-3-pyridyl-methylene)-N-(2-chloro-4-pyridyl)-N′-(3-trifluoromethyl-phenyl)-malonamide

The title compound was prepared from a mixture of N-(2-chloro-4-pyridyl)-N′-(3-trifluoromethyl-phenyl)-malonamide (100 mg, 0.28 mmol) and 6-chloro-3-pyridinecarboxyaldehyde (51 mg, 0.364 mmol) similar to Example 3 and isolated as a solid. Compound A (7 mg, 7%). ¹H NMR (acetone-d₆): 10.31 (s, 1H), 9.32 (s, 1H), 8.59 (d, 3=2.4 Hz, 1H), 8.29 (d, J=5.7 Hz, 1H), 8.05 (s, 1H), 8.03-7.88 (m, 5H), 7.61-7.45 (m, 4H). Compound B (23 mg, 23%). ¹H NMR (acetone-d₆): 10.19 (s, 1H), 9.95 (s, 1H), 8.59 (d, J=2.4 Hz, 1H), 8.28 (d, J=5.7 Hz, 1H), 8.04 (s, 1H), 8.02 (dd, J=2.4, 5.7 Hz, 1H), 7.92 (d, J=2.4 Hz, 1H), 7.88 (s, 1H), 7.77 (d, J=8.1 Hz, 1H), 7.62-7.48 (m, 5H).

### EXAMPLE 91

### (E) and (Z)-2-(4-Isopropyl-benzylidene)-N-(3-ethylcarboxyl-phenyl)-N′-(3-trifluoromethyl-phenyl)-malonamide

a) N-(3-ethylcarboxyl-phenyl)-N′-(3-trifluoromethyl-phenyl)-malonamide: To a stirring solution of N-(3-trifluoromethyl-phenyl)-malonamic acid (1 g, 4.05 mmol) in anhydrous dichloromethane (100 mL) was added phosphorus pentachloride (842 mg, 4.05 mmol). The mixture was stirred at room temperature for 30 minutes. Then 3-aminoethylbenzoate (802 mg, 4.86 mmol) was added and the mixture was stirred at room temperature for 20 h. The organic solvent was evaporated and the product was isolated as off white solid from a small silica column (790 mg, 55%). ¹H NMR (CDCl₃): 9.80 (s, 1H), 9.67 (s, 1H), 8.21 (s, 1H), 7.93-7.73 (m, 4H), 7.44-7.35 (m, 3H), 4.37(q, J=7.2 Hz, 2H), 3.71 (s, 2H), 1.32(t, J=7.2 Hz, 3H).

b) (E) and (Z)-2-(4-isopropyl-benzylidene)-N-(3-ethylcarboxy-phenyl)-N′-(3-trifluoromethyl-phenyl)-malonamide: The title compound was prepared from a mixture of N-(3-ethylcarboxyl-phenyl)-N′-(3-trifluoromethyl-phenyl)-malonamide (50 mg, 0.127 mmol) and 4-isopropylbenzaldehyde (16 μL, 0.165 mmol) similar to Example 3 and isolated as solid. Compound A (4 mg, 8%). ¹H NMR (CDCl₃): 9.95 (s, 1H), 8.18 (s, 1H), 8.11 (s, 1H), 7.97 (d, J=8.7 Hz, 1H), 7.84 (d, J=7.8 Hz, 1H), 7.58 (m, 1H), 7.49-7.36 (m, 4H), 7.28-7.24 (m, 2H), 7.19-7.18 (m, 2H), 4.38 (q, J=7.5 Hz, 2H), 2.87 (sept, J=6.9 Hz, 1H), 1.36 (t, J=7.5 Hz, 3H), 1.89 (d, J=6.9 Hz, 3H). Compound B (4 mg, 8%). ¹H NMR (CDCl₃): ¹H NMR (CDCl₃): 10.13 (s, 1H), 8.23 (s, 1H), 8.00 (s, 1H), 7.99-7.90 (m, 3H), 7.75 (brd, J=7.8 Hz, 1H), 7.60-7.48 (m, 5H), 7.38-7.32 (m, 3H), 4.49 (q, J=7.2 Hz, 2H), 2.99 (sept, J=6.6 Hz, 1H), 1.47(t, J=7.2 Hz, 3H), 1.31 (d, J=6.6 Hz, 3H).

### EXAMPLE 92

### N,N′-Bis-(3-methoxy-5-trifluoromethyl-phenyl)-2-(4-isopropyl-benzylidene)-malonamide

a) N,N′-Bis-(3-methoxy-5-trifluoromethylphenyl)malonamide: To a stirring solution of 3-methoxy-5-trifluoromethylaniline (500 mg, 2.62 mmol) in 1,4-dioxane (10 mL) was slowly added malonyl dichloride (130 μL, 1.31 mmol). The mixture was stirred at room temperature for 30 min. The mixture was diluted with water and the resulting solid was collected by filtration. The solid was washed with excess water and then dried under vacuum at 45° C. to give a white solid (400, 68%). ¹H NMR (CDCl₃): 9.10 (s, 2H), 7.56 (s, 2H), 7.50 (s, 2H), 7.05 (s, 2H), 3.96 (s, 6H), 3.67 (s, 2H).

b) N,N′-Bis-(3-methoxy-5-trifluoromethyl-phenyl)-2-(4-isopropyl-benzylidene)-malonamide: The title compound was prepared from a mixture of N,N′-bis-(3-methoxy-5-trifluoromethyl-phenyl)malonamide (100 mg, 0.222 mmol) and isopropylbenzaldehyde (41 μL, 0.333 mmol) similar to Example 3 and isolated as a white solid (10 mg, 14%). ¹H NMR (CDCl₃): 10.15 (s, 1H), 8.99 (s, 1H), 7.92 (s, 1H), 7.50 (s, 1H), 7.30 (brd, J=4.8 Hz, 2H), 7.29-7.26 (m, 3H), 7.06 (d, J=8.4 Hz, 2H), 6.95 (s, 1H), 6.91 (s, 1H), 3.85 (s, 3H), 3.80 (s, 3H), 2.78 (sept, J=6.9 Hz, 1H), 1.12 (d, J=6.9 Hz, 6H).

### EXAMPLE 93

### (E) and (Z)-2-(6-Trifluoromethyl-3-pyridyl-methylene)-N-(quinolin-6-yl)-N′-(3-trifluoromethyl-phenyl)malonamide

a) N-(quinolin-6-yl)-N′-(3-trifluoromethyl-phenyl)-malonamide: To a stirring solution of N-(3-trifluoromethyl-phenyl)-malonamic acid (1 g, 4.05 mmol) in anhydrous dichloromethane (100 mL) was added phosphorus pentachloride (842 mg, 4.05 mmol). The mixture was stirred at room temperature for 30 minutes. Then 6-aminoquinoline (874 mg, 6.0 mmol) was added and the mixture was stirred at room temperature for 20 h. The reaction mixture was diluted with 50 ml dichloromethane and washed with 5% sodium bicarbonate. The organic layer was evaporated and the product was isolated as a white solid from a silica column (790 mg, 79%). ¹H NMR (CDCl₃): 9.80 (s, 1H), 9.67 (s, 1H), 8.21 (s, 1H), 7.93-7.73 (m, 4H), 7.44-7.35 (m, 3H), 4.37(q, J=7.2 Hz, 2H), 3.71 (s, 2H), 1.32(t, J=7.2 Hz, 3H).

b) (E) and (Z)-2-(6-Trifluoromethyl-3-pyridyl-methylene)-N-(quinolin-6-yl)-N′-(3-trifluoromethyl-phenyl)malonamide: The title compound was prepared from a mixture of N-(quinolin-6-yl)-N′-(3-trifluoromethyl-phenyl)malonamide (200 mg, 0.536 mmol) and 6-trifluoromethyl-3pyridinecarboxyaldehyde (140 mg, 0.8 mmol) similar to Example 3 and isolated as a solid. Compound A (7 mg, 3.5%). ¹H NMR (CDCl₃): 10.13 (s, 1H), 8.23 (s, 1H), 8.00 (s, 1H), 7.99-7.90 (m, 3H), 7.75 (brd, J=7.8 Hz, 1H), 7.60-7.48 (m, 5H), 7.38-7.32 (m, 3H), 4.49 (q, J=7.2 Hz, 2H), 2.99 (sept, J=6.6 Hz, 1H), 1.47(t, J=7.2 Hz, 3H), 1.31 (d, J=6.6 Hz, 3H). Compound B (8 mg, 4%) ¹H NMR (CDCl₃): 9.81 (s, 1H), 8.07 (s, 1H), 7.73 (s, 1H), 7.63 (d, J=7.2 Hz, 2H), 7.43 (d, J=8.1 Hz, 2H), 7.38-7.32 (m, 5H), 7.21 (d, J=8.4 Hz, 2H), 7.18-7.15 (m, 1H), 7.12 (d, J=7.5 Hz, 2H), 2.91-2.86 (m, 1H), 1.23 (s, 3H), 1.21 (s, 3H).

### EXAMPLE 94

### (E) and (Z)-2-(6-Trifluoromethyl-3-pyridyl-methylene)-N-(3-pyridyl)-N′-(3-trifluoromethyl-phenyl)-malonamide

a) N-(3-Pyridyl)-N′-(3-trifluoromethyl-phenyl)-malonamide: The title compound was from a mixture of N-(3-trifluoromethyl-phenyl)-malonamic acid (300 mg, 1.2 mmol) and 3-aminopyridine (108 mg, 1.2 mmol) similar to example 89 and the product was isolated as an off white solid (55 mg, 51%). ¹H NMR (CDCl₃): 9.69 (s, 1H), 8.69 (s, 1H), 8.31 (d, J=5.7 Hz, 1H), 7.92 (s, 1H), 7.73 (d, J=1.8 Hz, 1H), 7.71-7.67 (m, 1H), 7.53-7.44 (m, 2H), 7.39 (dd, J=2.1, 5.4 Hz, 1H), 3.61 (s, 2H).

b) (E) and (Z)-2-(6-Trifluoromethyl-3-pyridyl-methylene)-N-(3-pyridyl)-N′-(3-trifluoromethyl-phenyl)-malonamide: The title compound was prepared from a mixture of N-(3-pyridyl)-N′-(3-trifluoromethyl-phenyl)malonamide (50 mg, 0.155 mmol) and 6-trifluoromethyl-3-pyridinecarboxyaldehyde (40 mg, 0.225 mol) similar to Example 3 and isolated as a solid. Compound A (4 mg, 8%). ¹H NMR (CDCl₃): 9.72 (s, 1H), 8.75 (d, J=8.1 Hz 1H), 8.61 (s, 1H), 8.56 (d, J=5 Hz, 1H), 7.97-7.93(m, 3H), 7.69 (s, 1H), 7.67-7.48 (m, 6H). Compound B (4 mg, 8%). ¹H NMR (CDCl₃): 9.42 (s, 1H), 8.75 (d, J=8.1 Hz 1H), 8.61 (s, 1H), 8.56 (d, J=5 Hz, 1H), 8.01-7.9.6(m, 3H), 7.69 (s, 1H), 7.67-7.48(m, 6H).

### EXAMPLE 95

### (E) and (Z)-2-(4-Isopropyl-benzylidene)-N-(3-sulfamoyl-phenyl)-N′-(3-trifluoromethyl-phenyl)-malonamide

a) N-(3-Sulfamoyl-phenyl)-N′-(3-trifluoromethyl-phenyl)-malonamide: The title compound was prepared from a mixture of N-(3-trifluoromethyl-phenyl)-malonamic acid (70 mg, 0.283 mmol) and 3-aminophenylsulfonamide (31 mg, 0.425 mmol) similar to example 89 and the product was isolated as an off white solid (90 mg, 79%). ¹H NMR (acetone-d₆): 9.30 (s, 1H), 8.93 (s, 1H), 8.68 (d, J=8.1 Hz, 1H), 8.05 (d, J=8.4 Hz, 1H), 7.86-7.83 (m, 2H), 7.69-7.56 (m, 2H), 6.71 (s, 1H), 2.05 (s, 2H).

b) (E) and (Z)-2-(4-Isopropyl-benzylidene)-N-(3-sulfamoyl-phenyl)-N′-(3-trifluoromethyl-phenyl)malonamide: The title compound was prepared from a mixture of N-(3-sulfamoyl-phenyl)-N′-(3-trifluoromethyl-phenyl)malonamide (80 mg, 0.2 mmol) and 4-isopropylbenzaldehyde (40 μL, 300 mmol) similar to Example 3 and isolated as a mixture of E/Z isomers (4 mg, 5%). ¹H NMR (CDCl₃): 9.58 (s, 1H), 9.31 (s, 1H), 8.09 (s, 1H), 7.73-7.34 (m, 9H), 7.31 (m, 2H), 7.04-6.97 (m, 1H), 2.67 (m, 1H), 1.35(brd, 6H).

### EXAMPLE 96

### N,N′-Bis-(3-trifluoromethyl-4-N-morpholinylphenyl)-2-(3,5-dichloro-benzylidene)-malonamide

a) N,N′-Bis-(3-trifluoromethyl-4-N-morpholinylphenyl)malonamide: To a stirring solution of 3-trifluoromethyl-4-N-morpholinoaniline (500, 2.0 mmol) in 1,4-dioxane (10 mL) was slowly added malonyl dichloride (140 μL, 1.0 mmol). The mixture was stirred at room temperature for 30 min. The mixture was diluted with water and the resulting solid was collected by filtration. The solid was washed with excess water and then dried under vacuum at 45° C. to give a white solid (200 mg, 40%). ¹H NMR (CDCl₃): 9.71 (s, 2H), 8.87 (s, 2H), 7.52 (dd, J=9.0 and 5.2 Hz, 2H), 7.36-7.38 (m, 2H), 4.10-4.07 (t, J=4.5 Hz, 4H), 3.72 (s, 2H), 3.04-3.01 (m, 4H).

b) N,N′-Bis-(3-trifluoromethyl-4-N-morpholinylphenyl)-2-(3,5-dichloro-benzylidene)-malonamide: The title compound was prepared from a mixture of N,N′-bis-(3-trifluoromethyl-4-N-morpholinylphenyl)malonamide (50 mg, 0.128 mmol) and 3,5-dichlorobenzaldehyde (120 mg, 0.214 mmol) similar to Example 3 and isolated as a white solid (60 mg, 50%). ¹H NMR (CDCl₃): 10.98 (s, 1H), 8.99 (s, 1H), 8.87 (dd, J=9.0 and 2.1 Hz, 2H), 8.13 (s, 1H), 7.42-7.19 (m, 8H), 4.10-4.07 (t, J=4.2 Hz, 4H), 3.75 (t, J=4.2 Hz, 4H), 2.95 (m, 4H), 2.58 (m, 4H).

### EXAMPLE 97

### N,N′-Bis-(3-chloro-phenyl)-2-(3-trifluoromethyl-benzylidine)-malonamide

The title compound was prepared from a mixture of N,N′-bis-(3-chloro-phenyl)-malonamide (50 mg, 0.155 mmol) and 3-trifluoromethylbenzaldehyde (30 mg, 0.232 mmol) similar to Example 3 and isolated as a white solid (24 mg, 48%). ¹H NMR (CDCl₃): 9.98 (s, 1H), 9.14 (s, 1H), 7.79 (s, 1H), 7.70 (m, 2H), 7.59 (s, 1H), 7.52 (d, J=7.5 Hz, 2H), 7.35-7.19 (m, 7H).

### EXAMPLE 98

### N,N′-Bis-(3-chloro-phenyl)-2-(6-trifluoromethyl-3-pyridyl-methylene)-malonamide

The title compound was prepared from a mixture of N,N′-bis-(3-chloro-phenyl)-malonamide (50 mg, 0.155 mmol) and 6-trifluoromethyl-3-pyridinecarboxyaldehyde (40 mg, 0.232 mmol) similar to Example 3 and isolated as a white solid (15 mg, 30%). ¹H NMR (CDCl₃): 9.67 (s, 1H), 9.02 (s, 1H), 8.69 (d, J=1.5 Hz, 1H), 7.85 (dd, J=8.4 and 2.4 Hz, 1H), 7.75 (s, 1H), 7.11 (dd, J=6.0 and 3.9 Hz, 2H), 7.54 (d, J=7.8 Hz, 1H), 7.39-7.14 (m, 6H).

### EXAMPLE 99

### (E) and (Z)-2-(3,5-Dichloro-2-(2-morpholin-4-yl-ethoxy)-benzylidene)-N-(quinolin-6-yl)-N′-(3-trifluoromethyl-phenyl)malonamide

The title compound was prepared from a mixture of N-(quinolin-6-yl)-N′-(3-trifluoromethyl-phenyl)malonamide (115 mg, 0.308 mmol) and 3,5-dichloro-2-(2-morpholin-4-yl-ethoxy)-benzaldehyde (68 mg, 0.401 mmol) similar to Example 3 and isolated as a solid. Compound A (4 mg, 3.5%). ¹H NMR (CDCl₃): 10.13 (s, 1H), 9.82 (s, 1H), 8.87 (dd, J=4.5 and 1.8 Hz, 1H), 8.39 (d, J=2.7 Hz, 1H), 8.18 (d, J=7.2 Hz, 1H), 8.09 (d, J=9.1 Hz, 1H), 7.99 (s, 1H), 7.81 (s, 2H), 7.75 (dd, J=9.1, and 2.4 1H) 7.48-7.39 (m, 3H), 7.32(d, J=2.4 Hz, 1H), 7.19 (d, J=2.4 Hz, 1H), 4.09 (m, 2H), 3.55-3.52 (m, 4H), 2.79 (m, 2H), 2.49 (m, 4H). Compound B (4 mg, 3.5%). ¹H NMR (CDCl₃): 10.42 (s, 1H), 9.69 (s, 1H), 8.86 (brd, J=7.5 Hz, 2H), 8.38 (d, J=7.5 Hz, 2H), 8.14 (m, 2H), 7.97 (s, 2H), 7.84-7.60 (m, 5H), 4.26 (m, 2H), 3.66 (m, 4H), 2.76 (m, 2H), 2.48 (m, 4H).

### EXAMPLE 100

### (E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide

The title compound was prepared from a mixture N-[4-(2-morpholin-4-yl-ethoxy)-3-trifluoromethyl-phenyl]-N′-(3-trifluoromethyl-phenyl)-malonamide (100 mg, 0.193 mmol) and 6-trifluoromethyl-3-pyridinecarboxyaldehyde (50 mg, 0.290 mmol) similar to Example 3 and isolated as a solid. Compound A (5 mg, 5%). ¹H NMR (CDCl₃): 9.17 (s, 1H), 8.69 (s, 1H), 7.99 (s, 1H), 7.97 (s, 1H), 7.84 (dd, J=8.4 and 2.4 Hz, 1H), 7.77-7.67 (m, 2H), 7.61 (dd, J=9.0 and 2.7 Hz, 1H), 7.57-7.46(m, 4H), 6.98(d, J=9.0 Hz, 1H), 4.21 (t, J=5.7 Hz, 2H), 3.74-3.68 (m, 4H), 2.84 (t, J=5.7 Hz, 2H), 2.64-2.55 (m, 4H). Compound B (5 mg, 5%). ¹H NMR (CDCl₃): 9.69 (s, 1H), 9.36 (s, 1H), 8.69 (s, 1H), 7.93 (s, 1H), 7.88-7.83 (m, 2H), 7.76 (s, 1H), 7.67-7.65(m, 3H), 7.54-7.49(m, 3H), 6.97 (d, J=9.1 Hz, 1H), 4.21 (t, J=5.4 Hz, 2H), 3.75-3.71 (m, 4H), 2.86 (t, J=5.4 Hz, 2H), 2.64-2.61(m, 4H)

### EXAMPLE 101

### (E) and (Z) 2-(3,5-Dichloro-2-(2-morpholin-4-yl-ethoxy)-benzylidene)-N-(2-chloro-4-pyridyl)-N′-(3-trifluoromethyl-phenyl)-malonamide

The title compound was prepared from a mixture of N-(2-chloro-4-pyridyl)-N′-(3-trifluoromethyl-phenyl)malonamide (100 mg, 0.280 mmol) and 3,5-dichloro-2-(2-morpholin-4-yl-ethoxy)-benzaldehyde (127 mg, 0.420 mmol) similar to Example 3 and isolated as a solid. Compound A (3 mg, 3%). ¹H NMR (CDCl₃): 10.42 (s, 1H), 10.27 (s, 1H), 8.30 (d, J=5.7 Hz, 1H), 8.12 (s, 1H), 8.04 (s, 1H), 7.78 (d, J=7.8 Hz, 1H), 7.70 (d, J=1.5 Hz, 1H), 7.57-7.46 (m, 2H), 7.32 (dd J=5.7 and 2.1 Hz, 1H), 7.21 (d, J=2.1 Hz, 1H), 7.06 (d, J=2.4 Hz, 1H), 4.00 (t, J=5.4 Hz, 2H), 3.45-3.42 (m, 4H), 2.48 (t, J=5.4 Hz, 2H), 2.26-2.23 (m, 4H). Compound B (3 mg, 3%). ¹H NMR (CDCl₃): 10.30 (s, 1H), 9.76 (s, 1H), 8.92 (s, 1H), 8.32 (d, J=5.4 Hz, 1H), 7.98 (s, 1H), 7.85 (s, 1H), 7.75 (d, J=5.4 Hz, 1H), 7.70 (s, 1H), 7.52-7.43 (m, 2H), 7.42 (dd J=5.4 and 2.1 Hz, 1H), 7.33 (d, J=2.4 Hz, 1H), 7.15 (d, J=2.4 Hz, 1H), 4.09 (t, J=5.4 Hz, 2H), 3.51-3.48 (m, 4H), 2.70 (t, J=5.4 Hz, 2H), 2.44-2.42 (m, 4H).

### EXAMPLE 102

### (E) and (Z) 2-(6-Trifluoromethyl-3-pyridyl-methylene)-N-(3-carboxy-phenyl)-N′-(3-trifluoromethyl-phenyl)-malonamide

The title compound was prepared from a mixture N-(3-carboxy-phenylphenyl)-N′-(3-trifluoromethyl-phenyl)-malonamide (100 mg, 0.264 mmol) and 6-trifluoromethyl-3-pyridinecarboxyaldehyde (70 mg, 0.4 mmol) similar to Example 3 and isolated as a solid. Compound A (5 mg, 5%). ¹H NMR (acetone-d₆): 10.07 (s, 1H), 9.87 (s, 1H), 8.92 (s, 1H), 8.33 (s, 1H), 8.27-8.24 (m, 2H), 7.99-7.81 (m, 6H), 7.61 (t, J=4.8 Hz, 1H), 7.48 (m, 2H). Compound B (5 mg, 5%). ¹H NMR (acetone-dr): 10.07 (s, 1H), 9.67(s, 1H), 8.78 (s, 1H), 8.28 (s, 1H), 8.11 (d, J=7.5 Hz, 1H), 7.99 (s, 1H), 7.90-7.66 (m, 5H), 7.47-7.34 (m, 3H).

### EXAMPLE 103

### (E) and (Z) 2-(6-Trifluoromethyl-3-pyridyl-methylene)-N-[3-(morpholine-4-sulfonyl)-phenyl]-N′-(3-trifluoromethyl-phenyl)-malonamide

a) 4-(3-Nitro-benzenesulfonyl)-morpholine: To a solution of 3-nitro-benzenesulfonyl chloride (2.0 gm, 9.64 mmol) in DCM (50 mL) was added morpholine (1.06 gm, 10.6 mmol) and triethylamine (1.4 mL, 10.6 mmol). The mixture was stirred at room temperature for one hour. The reaction was diluted with dichloromethane (100 mL) and washed with water. The organic layer was dried over anhydrous sodium sulfate and concentrated under reduced pressure to a white solid (2.2 gm, 84%).

b) 3-(Morpholine-4-sulfonyl)-phenylamine: To a solution 4-(3-nitro-benzenesulfonyl)-morpholine (2.0 gm, 0.735 mmol) in methanol (80 mL) was added palladium (5 wt % on activated carbon). The mixture was hydrogenated at 50 psi for overnight. The mixture was filtered over celite and washed with methanol. The solvent was evaporated to dryness to obtain an off white solid (1.7 g, 85%).

c) N-[3-(morpholine-4-sulfonyl)-phenyl]-N′-(3-trifluoromethyl-phenyl)-malonamide: The title compound was prepared from a mixture of N-(3-trifluoromethyl-phenyl)-malonamic acid (500 mg, 2.04 mmol) and 3-(morpholine-4-sulfonyl)-phenylamine (600 mg, 2.5 mmol) similar to example 89 and the product was purified as a white solid after chromatography (550 mg, 91%). ¹H NMR (CDCl₃): 9.57 (s, 1H), 9.26 (s, 1H), 8.07 (s, 1H), 7.95 (s, 1H), 7.86-7.83 (m, 1H), 7.74 (d, J=7.8 Hz, 1H), 7.57-7.48 (m, 2H), 7.45-7.41 (m, 2H), 3.73 (t, J=4.8 Hz, 4H), 3.03 (t, J=4.8 Hz, 4H).

d) (E) and (Z) 2-(6-Trifluoromethyl-3-pyridyl-methylene)-N-[3-(morpholine-4-sulfonyl)-phenyl]-N′-(3-trifluoromethyl-phenyl)-malonamide: The title compound was prepared from a mixture N-[3-(morpholine-4-sulfonyl)-phenyl]-N′-(3-trifluoromethyl-phenyl)-malonamide (250 mg, 0.533 mmol) and 6-trifluoromethyl-3-pyridinecarboxyaldehyde (139 mg, 0.794 mmol) similar to Example 3 and isolated as a white solid. Compound A (8 mg, 3%). ¹H NMR (CDCl₃): 9.85 (s, 1H), 9.76 (s, 1H), 8.65 (d, J=1.5 Hz, 1H), 7.92-7.84 (m, 3H), 7.83 (dd, J=8.4 and 2.1 Hz, 1H), 7.67 (s, 1H), 7.74 (s, 1H), 7.59-7.43 (m, 5H), 3.73 (t, J=4.5 Hz, 4H), 3.02 (t, J=4.5 Hz, 4H). Compound B (5 mg, 2%). ¹H NMR (CDCl₃): 9.91 (s, 1H), 9.52 (s, 1H), 8.68 (d, J=1.5 Hz, 1H), 8.12 (s, 1H), 7.93 (dd, J=8.1 and 2.1 Hz, 1H), 7.88 (s, 1H), 7.82-7.80 (m, 2H), 7.66 (m, 1H), 7.57-7.45 (m, 5H), 3.70 (t, J=4.2 Hz, 4H), 2.98 (t, J=4.2 Hz, 4H).

### EXAMPLE 104

### (E) and (Z) 2-(3-Trifluoromethyl-benzylidene)-N-[3-(morpholine-4-sulfonyl)-phenyl]-N′-(3-trifluoromethyl-phenyl)-malonamide

The title compound was prepared from a mixture N-[3-(morpholine-4-sulfonyl)-phenyl]-N′-(3-trifluoromethyl-phenyl)-malonamide (250 mg, 0.533 mmol) and 3-trifluoromethylbenzaldehyde (121 mg, 0.693 mmol) similar to Example 3 and isolated as a white solid. Compound A (7.5 mg, 3%). ¹H NMR (CDCl₃): 9.94 (s, 1H), 9.16 (s, 1H), 8.02 (t, J=1.5 Hz, 1H), 7.93 (s, 1H), 7.83 (s, 1H), 7.77 (d, J=8.1 Hz, 1H), 7.69 (m, 1H), 7.59-7.43 (m, 7H), 7.29 (t, J=8.1 Hz, 1H), 3.75 (t, J=4.8 Hz, 4H), 3.03 (t, J=4.8 Hz, 4H). Compound B (8 mg, 3%). ¹H NMR (CDCl₃): 10.13 (s, 1H), 9.85 (s, 1H), 8.28 (s, 1H), 8.14 (s, 1H), 8.03 (dd, J=8.1 and 1.8 Hz, 1H), 7.94-7.87 (m, 3H), 7.78-7.55 (m, 7H), 3.69 (t, J=4.5 Hz, 4H), 2.99 (t, J=4.5 Hz, 4H).

### EXAMPLE 105

### Identification of N,N′-bis-(3-trifluoromethyl-phenyl)-2-(4-isopropyl-benzylidene)-malonamide and other Analogs as Antineoplastic Compounds that are Caspase Cascade Activators and Apoptosis Inducers

Human breast cancer cell lines T-47D and ZR-75-1 were grown according to media component mixtures designated by American Type Culture Collection +10% FCS (Invitrogen Corporation), in a 5% CO₂-95% humidity incubator at 37° C. T-47D and ZR-75-1 cells were maintained at a cell density between 30 and 80% confluency and for HL-60 at a cell density of 0.1 to 0.6×10⁶ cells/mL. Cells were harvested at 600×g and resuspended at 0.65×10⁶ cells/mL into appropriate media +10% FCS. An aliquot of 45 μL of cells was added to a well of a 96-well microtiter plate containing 5 μL of a 10% DMSO in RPMI-1640 media solution containing 1.6 to 100 μM of N,N′-bis-(3-trifluoromethyl-phenyl)-2-(4-isopropyl-benzylidene)-malonamide or other test compound (0.16 to 10 μM final). An aliquot of 45 μL of cells was added to a well of a 96-well microtiter plate containing 5 μL of a 10% DMSO in RPMI-1640 media solution without test compound as the control sample. The samples were mixed by agitation and then incubated at 37° C. for 24 h in a 5% CO₂-95% humidity incubator. After incubation, the samples were removed from the incubator and 50 mL of a solution containing 20 μM of N-(Ac-DEVD)-N′-ethoxycarbonyl-R110 fluorogenic substrate (SEQ ID NO:1) (Cytovia, Inc.; U.S. Pat. No. 6,335,429), 20% sucrose (Sigma), 20 mM DTT (Sigma), 200 mM NaCl (Sigma), 40 mM Na PIPES buffer pH 7.2 (Sigma), and 500 μg/mL lysolecithin (Calbiochem) was added. The samples were mixed by agitation and incubated at room temperature. Using a fluorescent plate reader (Model 1420 Wallac Instruments), an initial reading (T=0) was made approximately 1-2 min after addition of the substrate solution, employing excitation at 485 nm and emission at 530 nm, to determine the background fluorescence of the control sample. After approximately 3 h of incubation, the samples were read for fluorescence as above (T=3 h).

Calculation:

The relative Fluorescence Unit values (RFU) were used to calculate the sample readings as follows:
RFU_{(T=3h)}−Control RFU_{(T=0)}=Net RFU_{(T=3h)}

The activity of caspase cascade activation was determined by the ratio of the net RFU value for N,N′-bis-(3-trifluoromethyl-phenyl)-2-(4-isopropyl-benzylidene)-malonamide to that of control samples. The EC₅₀ (nM) was determined by a sigmoidal dose-response calculation (Prism 2.0, GraphPad Software Inc.). The caspase activity (Ratio) and potency (EC₅₀) are summarized in Table I:

TABLE I
Caspase Activity and Potency

| | |
|---|---|
| T-47D | ZR-75-1 |

| | | | | |
|---|---|---|---|---|
| The Compound | Ratio | EC₅₀ | Ratio | EC₅₀ |

| | | |
|---|---|---|
| of Example # | (nM) | (nM) |

| | | | | |
|---|---|---|---|---|
| 1 | 28 | 2850 | 14 | 5500 |
| 2 | 16 | 647 | 18 | 906 |
| 3 | 15 | 867 | 17 | 1404 |
| 4 | 28 | 5750 | 6 | 5600 |
| 5 | 30 | 1900 | 26 | 5400 |
| 6 | 1 | >10000 | 1 | >10000 |
| 7 | 1 | >10000 | 2.2 | 1700 |
| 8 | 17 | 4050 | 9.5 | 4600 |
| 10 | 16 | 5790 | 2.5 | 4500 |
| 11 | 1 | >10000 | 1 | >10000 |
| 12 | 1 | >10000 | 1 | >10000 |
| 13 | 21 | 2890 | 4.5 | 5500 |
| 14 | 21 | 738 | 7.2 | 1984 |
| 15 | 16 | 1380 | 6 | 2790 |

Thus, N,N′-bis-(3-trifluoromethyl-phenyl)-2-(4-isopropyl-benzylidene)-malonamide (Example 3) and other analogs are identified as potent caspase cascade activators and antineoplastic compounds in this assay.

### EXAMPLE 106

### Identification of N,N′-bis-(3-trifluoromethyl-phenyl)-2-(4-isopropyl-benzylidene)-malonamide as an Antineoplastic Compound that Inhibits Cell Proliferation (GI₅₀)

T-47D and DLD-1 cells were grown and harvested as in Example 105. An aliquot of 90 μL of cells (2.2×10⁴ cells/mL) was added to a well of a 96-well microtiter plate containing 10 μl of a 10% DMSO in RPMI-1640 media solution containing 1 nM to 100 μM of N,N′-bis-(3-trifluoromethyl-phenyl)-2-(4-isopropyl-benzylidene)-malonamide (0.1 nM to 10 μM final). An aliquot of 90 μL of cells was added to a well of a 96-well microtiter plate containing 10 μL of a 10% DMSO in RPMI-1640 media solution without compound as the control sample for maximal cell proliferation (A_{Max}). The samples were mixed by agitation and then incubated at 37° C. for 48 h in a 5% CO₂-95% humidity incubator. After incubation, the samples were removed from the incubator and 20 μL of CellTiter 96 AQ_{UEOUS} One Solution Cell Proliferation™ reagent (Promega) was added. The samples were mixed by agitation and incubated at 37° C. for 2-4 h in a 5% CO₂-95% humidity incubator. Using an absorbance plate reader (Model 1420 Wallac Instruments), an initial reading (T=0) was made approximately 1-2 min after addition of the solution, employing absorbance at 490 nm. This determines the possible background absorbance of the test compounds. No absorbance for N,N′-bis-(3-trifluoromethyl-phenyl)-2-(4-isopropyl-benzylidene)-malonamide was found at 490 nm. After the 2-4 h incubation, the samples were read for absorbance as above (A_{Test}).

Baseline for GI₅₀ (dose for 50% inhibition of cell proliferation) of initial cell numbers were determined by adding an aliquot of 90 μL of cells or 90 μL of media, respectively, to wells of a 96-well microtiter plate containing 10 μL of a 10% DMSO in RPMI-1640 media solution. The samples were mixed by agitation and then incubated at 37° C. for 0.5 h in a 5% CO₂-95% humidity incubator. After incubation, the samples were removed from the incubator and 20 μL of CellTiter 96 AQ_{UEOUS} One Solution Cell Proliferation™ reagent (Promega) was added. The samples were mixed by agitation and incubated at 37° C. for 2-4 h in a 5% CO₂-95% humidity incubator. Absorbance was read as above, (A_{Start}) defining absorbance for initial cell number used as baseline in GI₅₀ determinations.

Calculation:

GI₅₀ (dose for 50% inhibition of cell proliferation) is the concentration where [(A_{Test}−A_{Start})/(A_{Max}−A_{Start})]=0.5. The GI₅₀ (nM) are summarized in Table II:

TABLE II
GI₅₀ in Cancer Cells

| | |
|---|---|
| The Compound | GI₅₀ (nM) |

| of Example # | T-47D | DLD-1 |
|---|---|---|
| 2 | 150 | 200 |
| 3 | 180 | 200 |
| 8 | 1566 | 1528 |
| 14 | 1194 | 1230 |
| 15 | 46 | 1343 |
| 16 | 1722 | 1678 |
| 18 | 467 | 1652 |
| 20 | 1515 | 1659 |
| 26 | 666 | 1647 |

Thus, N,N′-bis-(3-trifluoromethyl-phenyl)-2-(4-isopropyl-benzylidene)-malonamide (Example 3) and analogs are identified as antineoplastic compound that inhibits cell proliferation.

### EXAMPLE 107

### N,N′-bis-(3-Trifluoromethyl-phenyl)-2-(4-isopropyl-benzylidene)-malonamide Induces Apoptosis in Jurkat Cells

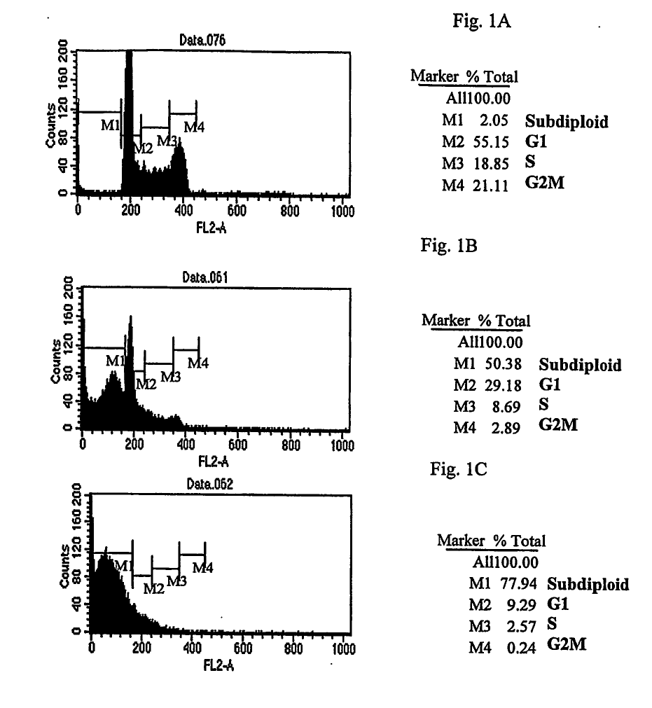

Jurkat cells were incubated with N,N′-bis-(3-trifluoromethyl-phenyl)-2-(4-isopropyl-benzylidene)-malonamide (0.5 μM and 1 μM) for 24 h under normal growth conditions; control cultures were treated with DMSO vehicle. Cells were harvested at 1200 rpm and washed twice with 5 mM EDTA/PBS. Cells were then resuspended in 300 μl EDTA/PBS and 700 ml of 100% ethanol, vortexed and incubated at room temperature for 1 h. Samples were spun down at 12000 rpm for 5 min and the supernatant was removed. A solution containing 100 μg/ml of propidium iodide and 1 mg/ml of RNAse A (fresh) was added to the samples and incubated for 1 h at room temperature. Samples were then transferred to 12×75 mm polystyrene tubes and analysed on a flow cytometer. All flow cytometry analyses were performed on FACScalibur (Becton Dickinson) using Cell Quest analysis software. FIGS. 1A-C show that N,N′-bis-(3-trifluoromethyl-phenyl)-2-(4-isopropyl-benzylidene)-malonamide induces massive apoptosis in Jurkat cells.

### EXAMPLE 108

### N,N′-bis-(3-Trifluoromethyl-phenyl)-2-(4-isopropyl-benzylidene)-malonamide Induces Apoptosis in T47D Cells

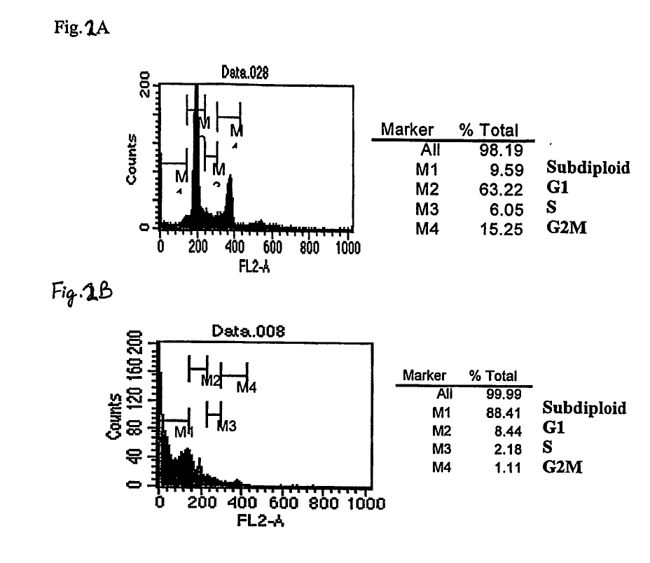

T47D breast cancer cell line was maintained and harvested as described in Example 105. 1×10⁶ cells were treated with 1.7 μM of N,N′-bis-(3-trifluoromethyl-phenyl)-2-(4-isopropyl-benzylidene)-malonamide for 24 h at 37° C. As a control, cells were also incubated with DMSO. Cells were harvested and treated with propidium iodide and analysed on a flow cytometer as described in Example 107. FIGS. 2A-B show that N,N′-bis-(3-trifluoromethyl-phenyl)-2-(4-isopropyl-benzylidene)-malonamide induces massive apoptosis in T47D Cells.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating or ameliorating a disorder responsive to the induction of apoptosis in an animal suffering therefrom, comprising administering to an animal in need of such treatment an effective amount of a compound of Formula I: 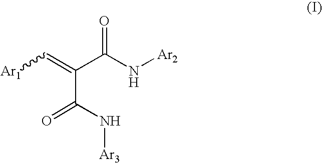
or a pharmaceutically acceptable salt or prodrug thereof, wherein:
Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

2. The method of claim 1, wherein Ar₁, Ar₂ and Ar₃ independently are optionally substituted phenyl, naphthyl, pyridyl, quinolyl, isoquinolyl, thienyl, furyl, pyrrolyl, indolyl, imidazolyl, pyrazolyl or cyclohexyl.

3. The method of claim 1, wherein Ar₁, Ar₂ and Ar₃ independently are optionally substituted and are phenyl or pyridyl.

4. The method of claim 1, wherein said compound is selected from the group consisting of:
2-[3-(4-Chloro-phenyl)-1-phenyl-1H-pyrazol-4-ylmethylene]-N,N′-bis-(3-trifluoromethyl-phenyl)-malonamide;
2-[3-(4-Methoxy-phenyl)-1-phenyl-1H-pyrazol-4-ylmethylene]-N,N′-bis-(3-trifluoromethyl-phenyl)-malonamide;
N,N′-Bis-(3-trifluoromethyl-phenyl)-2-(4-methyl-benzylidene)-malonamide;
N,N′-Bis-(3-trifluoromethyl-phenyl)-2-(4-bromo-benzylidene)-malonamide;
N,N′-Bis-(3-trifluoromethyl-phenyl)-2-(4-nitro-benzylidene)-malonamide;
N,N′-Bis-(3-trifluoromethyl-phenyl)-2-(3-chloro-benzylidene)-malonamide;
2-[3-(4-Fluoro-phenyl)-1-phenyl-1H-pyrazol-4-ylmethylene]-N,N′-bis-(3-trifluoromethyl-phenyl)-malonamide;
2-[1-phenyl-3-(thiophen-2-yl)-1H-pyrazol-4-ylmethylene]-N,N′-bis-(3-trifluoromethyl-phenyl)-malonamide;
N,N′-Bis-(3-methoxy-phenyl)-2-(4-trifluoromethyl-benzylidene)-malonamide;
N,N′-Bis-(3-trifluoromethyl-phenyl)-2-(2,4-dichloro-benzylidene)-malonamide;
N,N′-Bis-(3-trifluoromethyl-phenyl)-2-(4-isopropyl-benzylidene)-malonamide;
N,N′-Bis-(3-trifluoromethyl-phenyl)-2-furan-2-yl-methylene-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Bis-(3-trifluoromethyl-phenyl)-2-(4-pyridyl-methylene)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-trifluoromethyl-phenyl)-2-(3H-imidazol-4-yl-methylene)-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(4-ethoxy-benzylidene)-malonamide;
N,N′-Bis-(3-trifluoromethyl-phenyl)-2-(5-methyl-furan-2-yl-methylene)-malonamide;
N,N′-Bis-(3-methoxy-phenyl)-2-(4-chloro-2-nitro-benzylidene)-malonamide;
N,N′-Bis-(pyridin-3-yl)-2-(4-isopropyl-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Bis-(3-trifluoromethyl-phenyl)-2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
3-(4-Isopropyl-phenyl)-N-(3-trifluoromethyl-phenyl)-2-(3-trifluoromethyl-phenylsulfamoyl)-acrylamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Chloro-pyridin-4-yl)-2-(2,3-dihydro-benzo[1,4]dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
(E) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(6-bromo-pyridin-2-yl-methylene)malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(6-bromo-pyridin-2-yl)-2-(4-isopropyl-benzylidene)-malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide;
(E) and (Z)-2-(3-Trifluoromethyl-benzylidene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide;
or a pharmaceutically acceptable salt or prodrug thereof.

5. The method of claim 1, wherein said compound has the Formula II: 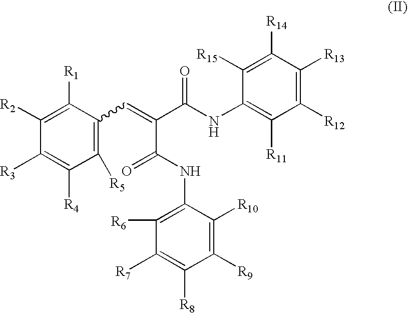
or a pharmaceutically acceptable salt or prodrug thereof, wherein R₁-R₁₅ are independently hydrogen, halo, haloalkyl, aryl, optionally substituted fused heteroaryl, carbocyclic, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocyclealkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate.

6. The method of claim 5, wherein said compound is selected from the group consisting of:
N,N′-Bis-(3-trifluoromethyl-phenyl)-2-(4-methyl-benzylidene)-malonamide;
N,N′-Bis-(3-trifluoromethyl-phenyl)-2-(4-bromo-benzylidene)-malonamide;
N,N′-Bis-(3-trifluoromethyl-phenyl)-2-(4-nitro-benzylidene)-malonamide;
N,N′-Bis-(3-trifluoromethyl-phenyl)-2-(3-chloro-benzylidene)-malonamide;
N,N′-Bis-(3-methoxy-phenyl)-2-(4-trifluoromethyl-benzylidene)-malonamide;
N,N′-Bis-(3-trifluoromethyl-phenyl)-2-(2,4-dichloro-benzylidene)-malonamide;
N,N′-Bis-(3-trifluoromethyl-phenyl)-2-(4-isopropyl-benzylidene)-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(4-ethoxy-benzylidene)-malonamide;
N,N′-Bis-(3-methoxy-phenyl)-2-(4-chloro-2-nitro-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(6-chloro-benzo[1,3]dioxol-5-yl-methylene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
3-(4-Isopropyl-phenyl)-N-(3-trifluoromethyl-phenyl)-2-(3-trifluoromethyl-phenylsulfamoyl)-acrylamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide; and
(E) and (Z)-2-(3-Trifluoromethyl-benzylidene)-N-[3-(morpholine-4-sulfonyl)-phenyl]-N′-(3-trifluoromethyl-phenyl)-malonamide;
or a pharmaceutically acceptable salt or prodrug thereof.

7. A method for treating or ameliorating cancer, comprising administering to an animal in need of such treatment an effective amount of a compound of Formula I: 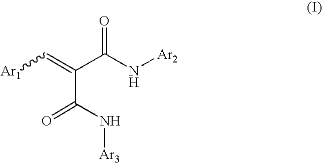
or a pharmaceutically acceptable salt or prodrug thereof, wherein:
Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

8. The method of claim 7, wherein said animal is a mammal.

9. The method of claim 7, wherein said cancer is selected from the group consisting of Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinomas, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma.

10. A method for the treatment or amelioration of drug-resistant cancer, comprising administering to an animal in need of such treatment or amelioration an effective amount of a compound of the Formula I: 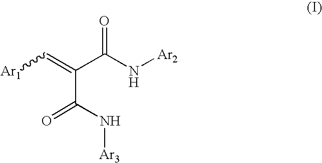
or a pharmaceutically acceptable salt or prodrug thereof, wherein:
Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

11. The method of claim 10, wherein said animal is a mammal.

12. The method of claim 7 or 10, additionally comprising administering at least one known cancer chemotherapeutic agent, or a pharmaceutically acceptable salt of said agent.

13. The method of claim 7 or 10, wherein said compound is administered together with at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylornithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

14. The method of claim 7 or 10, additionally comprising treating said animal with radiation-therapy.

15. The method of claim 1, wherein said disorder is rheumatoid arthritis.

16. The method of claim 1, wherein said disorder is inflammation.

17. The method of claim 1, wherein said disorder is inflamatory bowel disease.

18. The method of claim 1, wherein said disorder is Crohn's disease.

19. The method of claim 1, wherein said disorder is ulcerative colitis.

20. The method of claim 1, wherein said disorder is a skin disease.

21. The method of claim 20, wherein said disorder is psoriasis.

22. The method according to claim 1, wherein said disorder is an infectious viral disease.

23. A pharmaceutical composition comprising a pharmaceutically acceptable carrier and a compound of Formula I: 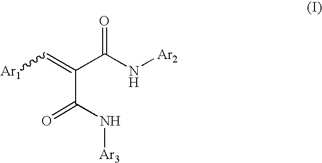
or a pharmaceutically acceptable salt or prodrug thereof, wherein:
Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

24. The pharmaceutical composition of claim 23, wherein said compound is selected from the group consisting of:
2-[3-(4-Chloro-phenyl)-1-phenyl-1H-pyrazol-4-ylmethylene]-N,N′-bis-(3-trifluoromethyl-phenyl)-malonamide;
2-[3-(4-Methoxy-phenyl)-1-phenyl-1H-pyrazol-4-ylmethylene]-N,N′-bis-(3-trifluoromethyl-phenyl)-malonamide;
N,N′-Bis-(3-trifluoromethyl-phenyl)-2-(4-methyl-benzylidene)-malonamide;
N,N′-Bis-(3-trifluoromethyl-phenyl)-2-(4-bromo-benzylidene)-malonamide;
N,N′-Bis-(3-trifluoromethyl-phenyl)-2-(4-nitro-benzylidene)-malonamide;
N,N′-Bis-(3-trifluoromethyl-phenyl)-2-(3-chloro-benzylidene)-malonamide;
2-[3-(4-Fluoro-phenyl)-1-phenyl-1H-pyrazol-4-ylmethylene]-N,N′-bis-(3-trifluoromethyl-phenyl)-malonamide;
2-[1-phenyl-3-(thiophen-2-yl)-1H-pyrazol-4-ylmethylene]-N,N′-bis-(3-trifluoromethyl-phenyl)-malonamide;
N,N′-Bis-(3-methoxy-phenyl)-2-(4-trifluoromethyl-benzylidene)-malonamide;
N,N′-Bis-(3-trifluoromethyl-phenyl)-2-(2,4-dichloro-benzylidene)-malonamide;
N,N′-Bis-(3-trifluoromethyl-phenyl)-2-(4-isopropyl-benzylidene)-malonamide;
N,N′-Bis-(3-trifluoromethyl-phenyl)-2-furan-2-yl-methylene-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Bis-(3-trifluoromethyl-phenyl)-2-(4-pyridyl-methylene)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-trifluoromethyl-phenyl)-2-(3H-imidazol-4-yl-methylene)-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(4-ethoxy-benzylidene)-malonamide;
N,N′-Bis-(3-trifluoromethyl-phenyl)-2-(5-methyl-furan-2-yl-methylene)-malonamide;
N,N′-Bis-(3-methoxy-phenyl)-2-(4-chloro-2-nitro-benzylidene)-malonamide;
N,N′-Bis-(pyridin-3-yl)-2-(4-isopropyl-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Bis-(3-trifluoromethyl-phenyl)-2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
3-(4-Isopropyl-phenyl)-N-(3-trifluoromethyl-phenyl)-2-(3-trifluoromethyl-phenylsulfamoyl)-acrylamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Chloro-pyridin-4-yl)-2-(2,3-dihydro-benzo[1,4]dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
(13) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(6-bromo-pyridin-2-yl-methylene)malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(6-bromo-pyridin-2-yl)-2-(4-isopropyl-benzylidene)-malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4N-morpholinylphenyl)-2-(3,5-dichloro-benzylidene)-malonamide;
N,N′-Bis-(3-chloro-phenyl)-2-(3-trifluoromethyl-benzylidene)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide;
(E) and (Z)-2-(3-Trifluoromethyl-benzylidene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide;
or a pharmaceutically acceptable salt or prodrug thereof.

25. The pharmaceutical composition of claim 23, additionally comprising at least one known cancer chemotherapeutic agent, or a pharmaceutically acceptable salt of said agent.

26. The pharmaceutical composition of claim 25, wherein said known cancer therapeutic agent is selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylornithine, ILX23-7553, fenretinide, N-4-carboxyphenyl retinamide, lactacystin, MG-132, PS-341, Gleevec®, ZD1839 Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

27. A compound of Formula I: 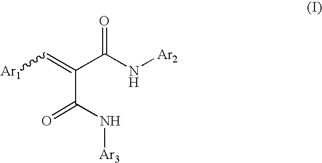
or a pharmaceutically acceptable salt or prodrug thereof, wherein:
Ar₁, Ar₂ and Ar₃ are independently and optionally substituted and are aryl, heteroaryl, saturated carbocyclic, partially saturated carbocylic, saturated heterocyclic, partially saturated heterocyclic, arylalkyl, or heteroarylalkyl.

28. The method of claim 27, wherein Ar₁, Ar₂ and Ar₃ independently are optionally substituted phenyl, naphthyl, pyridyl, quinolyl, isoquinolyl, thienyl, furyl, pyrrolyl, indolyl, imidazolyl, pyrazolyl or cyclohexyl.

29. The method of claim 27, wherein Ar₁, Ar₂ and Ar₃ independently are optionally substituted and are phenyl or pyridyl.

30. The compound of Formula II: 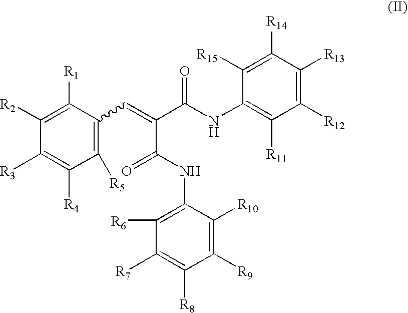
or a pharmaceutically acceptable salt or prodrug thereof, wherein:
R₁-R₁₅ are independently hydrogen, halo, haloalkyl, aryl, optionally substituted fused aryl, optionally substituted fused heteroaryl, carbocyclic, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocyclealkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate.

31. A compound selected from the group consisting of:
N,N′-Bis-(3-methoxy-phenyl)-2-(4-trifluoromethyl-benzylidene)-malonamide;
N,N′-Bis-(3-trifluoromethyl-phenyl)-2-furan-2-yl-methylene-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Bis-(3-trifluoromethyl-phenyl)-2-(4-pyridyl-methylene)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(4-trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-trifluoromethyl-phenyl)-2-(3H-imidazol-4-yl-methylene)-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(4-ethoxy-benzylidene)-malonamide;
N,N′-Bis-(3-trifluoromethyl-phenyl)-2-(5-methyl-furan-2-yl-methylene)-malonamide;
N,N′-Bis-(3-methoxy-phenyl)-2-(4-chloro-2-nitro-benzylidene)-malonamide;
N,N′-Bis-(pyridin-3-yl)-2-(4-isopropyl-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Bis-(3-trifluoromethyl-phenyl)-2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
3-(4-Isopropyl-phenyl)-N-(3-trifluoromethyl-phenyl)-2-(3-trifluoromethyl-phenylsulfamoyl)-acrylamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Chloro-pyridin-4-yl)-2-(2,3-dihydro-benzo[1,4]dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
(E) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(6-bromo-pyridin-2-yl-methylene)malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(6-bromo-pyridin-2-yl)-2-(4-isopropyl-benzylidene)-malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-4-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide;
(E) and (Z)-2-(3-Trifluoromethyl-benzylidene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide;
or a pharmaceutically acceptable salt or prodrugs thereof.

32. A compound selected from the group consisting of:
N,N′-Bis-(3-methoxy-phenyl)-2-(4-trifluoromethyl-benzylidene)-malonamide;
N,N′-Bis-(3-trifluoromethyl-phenyl)-2-(2,4-dimethyl-benzylidene)-malonamide;
N,N′-Bis-(3-trifluoromethyl-phenyl)-2-(2,4-dimethoxy-benzylidene)-malonamide;
N,N′-Bis-(3-trifluoromethyl-phenyl)-2-(4-isopropyl-benzyl)-malonamide;
N,N′-Diphenyl-2-(4-isopropyl-benzylidene)-malonamide;
N,N′-Bis-(2-trifluoromethyl-phenyl)-2-(4-isopropyl-benzylidene)-malonamide;
N,N′-Bis-(trifluoromethyl-phenyl)-2-(4-isopropyl-benzylidene)-malonamide;
N,N′-Bis-(3-methoxy-phenyl)-2-(4-isopropyl-benzylidene)-malonamide;
N,N′-Bis-(3-chloro-phenyl)-2-(4-isopropyl-benzylidene)-malonamide;
N,N′-Bis-(3-trifluoromethyl-phenyl)-2-benzylidene-malonamide;
N,N′-Bis-(3-nitro-phenyl)-2-(4-isopropyl-benzylidene)-malonamide;
N,N′-Bis-(3-trifluoromethyl-phenyl)-2-[(4-isopropyl-phenyl)-morpholin-4-yl-methyl]-malonamide;
N,N′-Bis-(3-bromo-phenyl)-2-(4-isopropyl-benzylidene)-malonamide;
N,N′-Bis-(3-trifluoromethyl-phenyl)-2-(4-ethoxy-benzylidene)-malonamide;
N,N′-Bis-(3-methoxy-phenyl)-2-(4-chloro-2-nitro-benzylidene)-malonamide;
N,N′-Bis-(3-trifluoromethyl-phenyl)-2-(4-ethyl-benzylidene)-malonamide;
N,N′-Bis-(3-trifluoromethyl-phenyl)-2-(4-methylsulfonyl-benzylidene)-malonamide;
N,N′-Bis-(3-trifluoromethyl-phenyl)-2-(4-cyano-benzylidene)-malonamide;
(E) and (Z)-2-(4-Isopropyl-benzylidene)-N-phenyl-N′-(3-trifluoromethyl-phenyl)-malonamide;
(E) and (Z)-2-(4-Isopropyl-benzylidene)-N-(3-methoxy-phenyl)-N′-(3-trifluoromethyl-phenyl)-malonamide;
N,N′-Bis-(3-trifluoromethyl-phenyl)-2-(4-isopropoxy-benzylidene)-malonamide;
N,N′-Bis-(3-trifluoromethyl-phenyl)-2-(2-chloro-4-hydroxy-benzylidene)-malonamide;
N,N′-Bis-(3-trifluoromethyl-phenyl)2-(4-pyrrolidin-1-yl-benzylidene)-malonamide;
N,N′-Bis-(3-trifluoromethyl-phenyl)-2-(3,4-dichloro-benzylidene)-malonamide;
N,N′-Bis-(5-chloro-2-hydroxy-phenyl)-2-(4-isopropyl-benzylidene)-malonamide;
N,N′-Bis-(3-trifluoromethyl-phenyl)-2-(2-chloro-4-dimethylamino-benzylidene)-malonamide;
N,N′-Bis-(3-trifluoromethyl-phenyl)-2-(2,5-dichloro-benzylidene)-malonamide;
N,N′-Bis-(3-trifluoromethyl-phenyl)-2-[3,5-dichloro-2-(2-morpholin-4-yl-ethoxy)-benzylidene]-malonamide;
N,N′-Bis-(3-trifluoromethyl-phenyl)-2-[5-chloro-2-(2-morpholin-4-yl-ethoxy)-benzylidene]-malonamide;
N,N′-Bis-(3-trifluoromethyl-phenyl)-2-(3,5-dimethoxy-benzylidene)-malonamide;
N,N′-Bis-(3-trifluoromethyl-phenyl)-2-(3,4,5-trimethoxy-benzylidene)-malonamide;
3-(4-Isopropyl-phenyl)-N-(3-trifluoromethyl-phenyl)-2-(3-trifluoromethyl-phenylsulfamoyl)-acrylamide;
(E) and (Z)-2-(4-Isopropyl-benzylidene)-N-[4-(2-morpholin-4-yl-ethoxy)-3-trifluoromethyl-phenyl]-N′-(3-trifluoromethyl-phenyl)-malonamide;
(E) and (Z)-N-(2-Dimethylamino-ethyl)-2-(4-isopropyl-benzylidene)-N′-(3-trifluoromethyl-phenyl)-malonamide;
(E) and (Z)-3-[3-(4-Isopropyl-phenyl)-2-(3-trifluoromethyl-phenylcarbamoyl)-acryloylamino]-benzoic acid;
N,N′-Bis-(3-trifluoromethyl-phenyl)-2-(4-dimethyamino-benzylidene)-malonamide;
N,N′-Bis-(3-trifluoromethyl-phenyl)-2-(3-nitro-5-chloro-benzylidene)-malonamide;
N,N′-Bis-(3-trifluoromethyl-phenyl)-2-(4-methoxy-benzylidene)-malonamide;
N,N′-Bis-(3-trifluoromethyl-phenyl)-2-(4-chloro-benzylidene)-malonamide;
N,N′-Bis-(3-trifluoromethyl-phenyl)-2-(3,5-dichloro-benzylidene)-malonamide;
N,N′-Bis-(3-trifluoromethyl-phenyl)-2-(2,6-dichloro-benzylidene)-malonamide;
N,N′-Bis-(3-trifluoromethyl-phenyl)-2-(2,3-dichloro-benzylidene)-malonamide;
N,N′-Bis-(3-trifluoromethyl-phenyl)-2-(4-N-methylpiperidinyl-benzylidene)-malonamide;
N,N′-Bis-(3-trifluoromethyl-phenyl)-2-(3-bromo-4,5-dimethoxy-benzylidene)-malonamide;
N,N′-Bis-(3-trifluoromethyl-phenyl)-2-(3,5-dimethoxy-4-hydroxy-benzylidene)-malonamide;
N,N′-Bis-(3-trifluoromethyl-phenyl)-2-(3-bromo-4-methoxy-benzylidene)malonamide;
N,N′-Bis-(3-carboxy-phenyl)-2-benzylidene-malonamide;
N,N′-Bis-(3-chloro-phenyl)-2-(3-chloro-5-nitro-benzylidene)-malonamide;
N,N′-Bis-(3-chloro-phenyl)-2-(3-bromo-4,5-dimethoxy-benzylidene)-malonamide;
(E) and (Z)-2-(4-Isopropyl-benzylidene)-N-(3-ethylcarboxyl-phenyl)-N′-(3-trifluoromethyl-phenyl)-malonamide;
N,N′-Bis-(3-methoxy-5-trifluoromethyl-phenyl)-2-(4-isopropyl-benzylidene)-malonamide;
(E) and (Z)-2-(4-Isopropyl-benzylidene)-N-(3-sulfamoyl-phenyl)-N′-(3-trifluoromethyl-phenyl)-malonamide;
N,N′-Bis-(3-trifluoromethyl-4-N-morpholinylphenyl)-2-(3,5-dichloro-benzylidene)-malonamide;
N,N′-Bis-(3-chloro-phenyl)-2-(3-trifluoromethyl-benzylidene)-malonamide; and
(E) and (Z)-2-(3-Trifluoromethyl-benzylidene)-N-[3-(morpholine-4-sulfonyl)-phenyl]-N′-(3-trifluoromethyl-phenyl)-malonamide;
or a pharmaceutically acceptable salt or prodrugs thereof.

33. A compound selected from the group consisting of:
N,N′-Bis-(3-trifluoromethyl-phenyl)-2-furan-2-yl-methylene-malonamide;
N,N′-Bis-(3-trifluoromethyl-phenyl)-2-(4-pyridyl-methylene)-malonamide;
N,N′-Bis-(3-trifluoromethyl-phenyl)-2-(3H-imidazol-4-yl-methylene)-malonamide;
N,N′-Bis-(3-trifluoromethyl-phenyl)-2-(6-methyl-pyridin-2-yl-methylene)-malonamide;
N,N′-Bis-(3-trifluoromethyl-phenyl)-2-(5-methyl-furan-2-yl-methylene)-malonamide;
N,N′-Bis-(pyridin-3-yl)-2-(4-isopropyl-benzylidene)-malonamide;
N,N′-Bis-(3-trifluoromethyl-phenyl)-2-(2-chloro-pyridin-3-yl-methylene)-malonamide;
N,N′-Bis-(3-trifluoromethyl-phenyl)-2-(6-chloro-benzo[1,3]dioxol-5-yl-methylene)-malonamide;
N,N′-Bis-(quinolin-6-yl)-2-(4-isopropyl-benzylidene)-malonamide;
(E) and (Z)-N-(6-Bromo-pyridin-2-yl)-2-(4-isopropyl-benzylidene)-N′-(3-trifluoromethyl-phenyl)-malonamide;
N,N′-Bis-(3-trifluoromethyl-phenyl)-2-(2,3-dihydro-benzofuran-5-yl-methylene)-malonamide;
N,N′-Bis-(3-trifluoromethyl-phenyl)-2-(2,3-dihydro-benzo[1,4]dioxin-6-yl-methylene)-malonamide;
N,N′-Di-(pyridin-2-yl)-2-(4-isopropyl-benzylidene)-malonamide;
N,N′-Bis-(3-trifluoromethyl-phenyl)-2-(5-methyl-pyridin-3-yl-methylene)-malonamide;
N,N′-Bis-(3-trifluoromethyl-phenyl)-2-(7-methoxy-benzo[1,3]dioxol-5-yl-methylene)-malonamide;
(E) and (Z)-N-(1H-Indol-5-yl)-2-(4-isopropyl-benzylidene)-N′-(3-trifluoromethyl-phenyl)-malonamide;
N,N′-Bis-(2-chloro-pyridin-4-yl)-2-(4-isopropyl-benzylidene)-malonamide;
N,N′-Bis-(3-trifluoromethyl-phenyl)-2-(6-trifluoromethyl-pyridin-3-yl-methylene)-malonamide;
(E) and (Z)-N-(2-Chloro-pyridin-4-yl)-2-(4-isopropyl-benzylidene)-N′-(3-trifluoromethyl-phenyl)-malonamide;
(E) and (Z)-N-(2-Chloro-pyridin-4-yl)-2-(2,3-dihydro-benzo[1,4] dioxin-6-yl-methylene)-N′-(3-trifluoromethyl-phenyl)-malonamide;
(E) and (Z)-2-Benzylidene-N-(2-chloro-pyridin-4-yl)-N′-(3-trifluoromethyl-phenyl)-malonamide;
(E) and (Z)-2-(4-Chloro-benzylidene)-N-(2-chloro-pyridin-4-yl)-N′-(3-trifluoromethyl-phenyl)-malonamide;
(E) and (Z)-N-(2-Chloro-1-oxy-pyridin-4-yl)-2-(4-isopropyl-benzylidene)-N′-(3-trifluoromethyl-phenyl)-malonamide;
N,N′-Bis-(3-trifluoromethyl-phenyl)-2-(5-bromo-2-furyl-methylene)-malonamide;
N,N′-Bis-(3-trifluoromethyl-phenyl)-2-(6-bromo-pyridin-2-yl-methylene)malonamide;
N,N′-Bis-(6-bromo-pyridin-2-yl)-2-(4-isopropyl-benzylidene)-malonamide;
(E) and (Z)-2-(3-Trifluoromethyl-benzylidene)-N-(2-chloro-4-pyridyl)-N′-(3-trifluoromethyl-phenyl)-malonamide;
(E) and (Z)-2-(3-Bromo-4,5-dimethoxy-benzylidene)-N-(2-chloro-4-pyridyl)-N′-(3-trifluoromethyl-phenyl)-malonamide;
(E) and (Z)-2-(3-Pyridyl-methylene)-N-(2-chloro-4-pyridyl)-N′-(3-trifluoromethyl-phenyl)-malonamide;
(E) and (Z)-2-(6-Chloro-3-pyridyl-methylene)-N-(2-chloro-4-pyridyl)-N′-(3-trifluoromethyl-phenyl)-malonamide;
(E) and (Z)-2-(6-Trifluoromethyl-3-pyridyl-methylene)-N-(quinolin-6-yl)-N′-(3-trifluoromethyl-phenyl)malonamide;
(E) and (Z)-2-(6-Trifluoromethyl-3-pyridyl-methylene)-N-(3-pyridyl)-N′-(3-trifluoromethyl-phenyl)-malonamide;
N,N′-Bis-(3-chloro-phenyl)-2-(6-trifluoromethyl-3-pyridyl-methylene)-malonamide;
(E) and (Z)-2-(3,5-Dichloro-2-(2-morpholin-4-yl-ethoxy)-benzylidene)-N-(quinolin-6-yl)-N′-(3-trifluoromethyl-phenyl)malonamide;
(E) and (Z)-2-(6-Trifluoromethyl-3-pyridyl-methylene)-N-[4-(2-morpholin-yl-ethoxy)-3-trifluoromethyl-phenyl]-N′-(3-trifluoromethyl-phenyl)-malonamide;
(E) and (Z)-2-(3,5-Dichloro-2-(2-morpholin-4-yl-ethoxy)-benzylidene)-N-(3-pyridyl)-N′-(3-trifluoromethyl-phenyl)-malonamide;
(E) and (Z)-2-(6-Trifluoromethyl-3-pyridyl-methylene)-N-(3-carboxy-phenyl)-N′-(3-trifluoromethyl-phenyl)-malonamide;
(E) and (Z)-2-(6-Trifluoromethyl-3-pyridyl-methylene)-N-[3-(morpholine-4-sulfonyl)-phenyl]-N′-(3-trifluoromethyl-phenyl)-malonamide; and
(E) and (Z)-2-(6-Trifluoromethyl-3-pyridyl-methylene)-N-(2-chloro-4-pyridyl)-N′-(3-trifluoromethyl-phenyl)malonamide;
or a pharmaceutically acceptable salt or prodrugs thereof.
